SEQUENCE LISTING (1123 sequences)

SEQ ID NO: 1 - DNA, 2280 bp, Homo sapiens
gccgccgcccccgcctgggccgcgctccccctctcccgctccctccctccctgctccaactcctcctccttctccatgcctctgttcctcctgctcttacttgtcctgctcctgctgctcgaggacgctggagcccagcaaggtgatggatgtggacacactgtactaggccctgagagtggaacccttacatccataaactacccacagacctatcccaacagcactgtttgtgaatgggagatccgtgtaaagatgggagagagagttcgcatcaaatttggtgactttgacattgaagattctgattcttgtcactttaattacttgagaatttataatggaattggagtcagcagaactgaaataggcaaatactgtggtctggggttgcaaatgaaccattcaattgaatcaaaaggcaatgaaatcacattgctgttcatgagtggaatccatgtttctggacgcggatttttggcctcatactctgttatagataaacaagatctaattacttgtttggacactgcatccaattttttggaacctgagttcagtaagtactgcccagctggttgtctgcttccttttgctgagatatctggaacaattcctcatggatatagagattcctcgccattgtgcatggctggtgtgcatgcaggagtagtgtcaaacacgttgggcggccaaatcagtgttgtaattagtaaaggtattccctattatgaaagttctttggctaacaacgtcacatctgtggtgggacacttatctacaagtctttttacatttaagacaagtggatgttatggaacactggggatggagtctggtgtgatcgcggatcctcaaataacagcatcatctgtgctggagtggactgaccacacagggcaagagaacagttggaaacccaaaaaagccaggctgaaaaaacctggaccgccttgggctgcttttgccactgatgaataccagtggttacaaatagatttgaataaggaaaagaaaataacaggcattataaccactggatccaccatggtggagcacaattactatgtgtctgcctacagaatcctgtacagtgatgatgggcagaaatggactgtgtacagagagcctggtgtggagcaagataagatatttcaaggaaacaaagattatcaccaggatgtgcgtaataactttttgccaccaattattgcacgttttattagagtgaatcctacccaatggcagcagaaaattgccatgaaaatggagctgctcggatgtcagtttattcctaaaggtcgtcctccaaaacttactcaacctccacctcctcggaacagcaatgacctcaaaaacactacagcccctccaaaaatagccaaaggtcgtgccccaaaatttacgcaaccactacaacctcgcagtagcaatgaatttcctgcacagacagaacaaacaactgccagtcctgatatcagaaatactaccgtaactccaaatgtaaccaaagatgtagcgctggctgcagttcttgtccctgtgctggtcatggtcctcactactctcattctcatattagtgtgtgcttggcactggagaaacagaaagaaaaaaactgaaggcacctatgacttaccttactgggaccgggcaggttggtggaaaggaatgaagcagtttcttcctgcaaaagcagtggaccatgaggaaaccccagttcgctatagcagcagcgaagttaatcacctgagtccaagagaagtcaccacagtgctgcaggctgactctgcagagtatgctcagccactggtaggaggaattgttggtacacttcatcaaagatctacctttaaaccagaagaaggaaaagaagcaggctatgcagacctagatccttacaactcaccagggcaggaagtttatcatgcctatgctgaaccactcccaattacggggcctgagtatgcaaccccaatcatcatggacatgtcagggcaccccacaacttcagttggtcagccctccacatccactttcaaggctacggggaaccaacctcccccactagtgggaacttacaatacacttctctccaggactgacagctgctcctcagcccaggcccagtatgataccccgaaagctgggaagccaggtctacctgccccagacgaattggtgtaccaggtgccacagagcacacaagaagtatcaggagcaggaagggatggggaatgtgatgtttttaaagaaatcctttgaagatgatgctgcttt

SEQ ID NO: 2 - DNA, 2190 bp, Homo sapiens
atgcctctgttcctcctgctcttacttgtcctgctcctgctgctcgaggacgctggagcccagcaaggtgatggatgtggacacactgtactaggccctgagagtggaacccttacatccataaactacccacagacctatcccaacagcactgtttgtgaatgggagatccgtgtaaagatgggagagagagttcgcatcaaatttggtgactttgacattgaagattctgattcttgtcactttaattacttgagaatttataatggaattggagtcagcagaactgaaataggcaaatactgtggtctggggttgcaaatgaaccattcaattgaatcaaaaggcaatgaaatcacattgctgttcatgagtggaatccatgtttctggacgcggatttttggcctcatactctgttatagataaacaagatctaattacttgtttggacactgcatccaattttttggaacctgagttcagtaagtactgcccagctggttgtctgcttccttttgctgagatatctggaacaattcctcatggatatagagattcctcgccattgtgcatggctggtgtgcatgcaggagtagtgtcaaacacgttgggcggccaaatcagtgttgtaattagtaaaggtattccctattatgaaagttctttggctaacaacgtcacatctgtggtgggacacttatctacaagtctttttacatttaagacaagtggatgttatggaacactggggatggagtctggtgtgatcgcggatcctcaaataacagcatcatctgtgctggagtggactgaccacacagggcaagagaacagttggaaacccaaaaaagccaggctgaaaaaacctggaccgccttgggctgcttttgccactgatgaataccagtggttacaaatagatttgaataaggaaaagaaaataacaggcattataaccactggatccaccatggtggagcacaattactatgtgtctgcctacagaatcctgtacagtgatgatgggcagaaatggactgtgtacagagagcctggtgtggagcaagataagatatttcaaggaaacaaagattatcaccaggatgtgcgtaataactttttgccaccaattattgcacgttttattagagtgaatcctacccaatggcagcagaaaattgccatgaaaatggagctgctcggatgtcagtttattcctaaaggtcgtcctccaaaacttactcaacctccacctcctcggaacagcaatgacctcaaaaacactacagcccctccaaaaatagccaaaggtcgtgccccaaaatttacgcaaccactacaacctcgcagtagcaatgaatttcctgcacagacagaacaaacaactgccagtcctgatatcagaaatactaccgtaactccaaatgtaaccaaagatgtagcgctggctgcagttcttgtccctgtgctggtcatggtcctcactactctcattctcatattagtgtgtgcttggcactggagaaacagaaagaaaaaaactgaaggcacctatgacttaccttactgggaccgggcaggttggtggaaaggaatgaagcagtttcttcctgcaaaagcagtggaccatgaggaaaccccagttcgctatagcagcagcgaagttaatcacctgagtccaagagaagtcaccacagtgctgcaggctgactctgcagagtatgctcagccactggtaggaggaattgttggtacacttcatcaaagatctacctttaaaccagaagaaggaaaagaagcaggctatgcagacctagatccttacaactcaccagggcaggaagtttatcatgcctatgctgaaccactcccaattacggggcctgagtatgcaaccccaatcatcatggacatgtcagggcaccccacaacttcagttggtcagccctccacatccactttcaaggctacggggaaccaacctcccccactagtgggaacttacaatacacttctctccaggactgacagctgctcctcagcccaggcccagtatgataccccgaaagctgggaagccaggtctacctgccccagacgaattggtgtaccaggtgccacagagcacacaagaagtatcaggagcaggaagggatggggaatgtgatgtttttaaagaaatcctttga

SEQ ID NO: 3 - protein, 729 aa, Homo sapiens [sequence omitted]

SEQ ID NO: 4 - DNA, 300 bp, Homo sapiens
aaacagaaagaaaaaaactgaaggcacctatgacttaccttactgggaccgggcaggttggtggaaaggaatgaagcagtttcttcctgcaaaagcagtggaccatgaggaaaccccagttcgctatagcagcagcgaagttaatcacctgagtccaagagaagtcaccacagtgctgcaggctgactctgcagagtatgctcagccactggtaggaggaattgttggtacacttcatcaaagatctacctttaaaccagaagaaggaaaagaagcaggctatgcagacctagatcctta

SEQ ID NO: 5 - protein, 99 aa, Homo sapiens
NRKKKTEGTYDLPYWDRAGWWKGMKQFLPAKAVDHEETPVRYSSSEVNHLSPREVTTVLQADSAEYAQPLVGGIVGTLHQRSTFKPEEGKEAGYADLDP

SEQ ID NO: 6 - DNA, 275 bp, Homo sapiens
caacttcagttggtcagccctccacatccactttcaaggctacggggaaccaacctcccccactagtgggaacttacaatacacttctctccaggactgacagctgctcctcagcccaggcccagtatgataccccgaaagctgggaagccaggtctacctgccccagacgaattggtgtaccaggtgccacagagcacacaagaagtatcaggagcaggaagggatggggaatgtgatgtttttaaagaaatcctttgaagatgatgctgcttt

SEQ ID NO: 7 - DNA, 260 bp, Homo sapiens
caacttcagttggtcagccctccacatccactttcaaggctacggggaaccaacctcccccactagtgggaacttacaatacacttctctccaggactgacagctgctcctcagcccaggcccagtatgataccccgaaagctgggaagccaggtctacctgccccagacgaattggtgtaccaggtgccacagagcacacaagaagtatcaggagcaggaagggatggggaatgtgatgtttttaaagaaatcctttga

SEQ ID NO: 8 - DNA, 15 bp, Homo sapiens
agatgatgctgcttt

SEQ ID NO: 9 - protein, 85 aa, Homo sapiens
TSVGQPSTSTFKATGNQPPPLVGTYNTLLSRTDSCSSAQAQYDTPKAGKPGLPAPDELVYQVPQSTQEVSGAGRDGECDVFKEIL

SEQ ID NO: 10 - DNA, 142 bp, Homo sapiens
gccgccgcccccgcctgggccgcgctccccctctcccgctccctccctccctgctccaactcctcctccttctccatgcctctgttcctcctgctcttacttgtcctgctcctgctgctcgaggacgctggagcccagcaag

SEQ ID NO: 11 - DNA, 228 bp, Homo sapiens
gtgatggatgtggacacactgtactaggccctgagagtggaacccttacatccataaactacccacagacctatcccaacagcactgtttgtgaatgggagatccgtgtaaagatgggagagagagttcgcatcaaatttggtgactttgacattgaagattctgattcttgtcactttaattacttgagaatttataatggaattggagtcagcagaactgaaatag

SEQ ID NO: 12 - DNA, 138 bp, Homo sapiens
gcaaatactgtggtctggggttgcaaatgaaccattcaattgaatcaaaaggcaatgaaatcacattgctgttcatgagtggaatccatgtttctggacgcggatttttggcctcatactctgttatagataaacaag

SEQ ID NO: 13 - DNA, 52 bp, Homo sapiens
atctaattacttgtttggacactgcatccaattttttggaacctgagttcag

SEQ ID NO: 14 - DNA, 73 bp, Homo sapiens
taagtactgcccagctggttgtctgcttccttttgctgagatatctggaacaattcctcatggatatagagat

SEQ ID NO: 15 - DNA, 134 bp, Homo sapiens
tcctcgccattgtgcatggctggtgtgcatgcaggagtagtgtcaaacacgttgggcggccaaatcagtgttgtaattagtaaaggtattccctattatgaaagttctttggctaacaacgtcacatctgtggt

SEQ ID NO: 16 - DNA, 41 bp, Homo sapiens
gggacacttatctacaagtctttttacatttaagacaagtg

SEQ ID NO: 17 - DNA, 216 bp, Homo sapiens
gatgttatggaacactggggatggagtctggtgtgatcgcggatcctcaaataacagcatcatctgtgctggagtggactgaccacacagggcaagagaacagttggaaacccaaaaaagccaggctgaaaaaacctggaccgccttgggctgcttttgccactgatgaataccagtggttacaaatagatttgaataaggaaaagaaaataacag

SEQ ID NO: 18 - DNA, 125 bp, Homo sapiens
gcattataaccactggatccaccatggtggagcacaattactatgtgtctgcctacagaatcctgtacagtgatgatgggcagaaatggactgtgtacagagagcctggtgtggagcaagataag

SEQ ID NO: 19 - DNA, 151 bp, Homo sapiens
atatttcaaggaaacaaagattatcaccaggatgtgcgtaataactttttgccaccaattattgcacgttttattagagtgaatcctacccaatggcagcagaaaattgccatgaaaatggagctgctcggatgtcagtttattcctaaag

SEQ ID NO: 20 - DNA, 87 bp, Homo sapiens
gtcgtcctccaaaacttactcaacctccacctcctcggaacagcaatgacctcaaaaacactacagcccctccaaaaatagccaaag

SEQ ID NO: 21 - DNA, 126 bp, Homo sapiens
gtcgtgccccaaaatttacgcaaccactacaacctcgcagtagcaatgaatttcctgcacagacagaacaaacaactgccagtcctgatatcagaaatactaccgtaactccaaatgtaaccaaag

SEQ ID NO: 22 - DNA, 94 bp, Homo sapiens
atgtagcgctggctgcagttcttgtccctgtgctggtcatggtcctcactactctcattctcatattagtgtgtgcttggcactggagaaacag

SEQ ID NO: 23 - DNA, 50 bp, Homo sapiens
aaagaaaaaaactgaaggcacctatgacttaccttactgggaccgggcag

SEQ ID NO: 24 - DNA, 138 bp, Homo sapiens
gttggtggaaaggaatgaagcagtttcttcctgcaaaagcagtggaccatgaggaaaccccagttcgctatagcagcagcgaagttaatcacctgagtccaagagaagtcaccacagtgctgcaggctgactctgcag

SEQ ID NO: 25 - DNA, 485 bp, Homo sapiens
agtatgctcagccactggtaggaggaattgttggtacacttcatcaaagatctacctttaaaccagaagaaggaaaagaagcaggctatgcagacctagatccttacaactcaccagggcaggaagtttatcatgcctatgctgaaccactcccaattacggggcctgagtatgcaaccccaatcatcatggacatgtcagggcaccccacaacttcagttggtcagccctccacatccactttcaaggctacggggaaccaacctcccccactagtgggaacttacaatacacttctctccaggactgacagctgctcctcagcccaggcccagtatgataccccgaaagctgggaagccaggtctacctgccccagacgaattggtgtaccaggtgccacagagcacacaagaagtatcaggagcaggaagggatggggaatgtgatgtttttaaagaaatcctttgaagatgatgctgcttt

SEQ ID NO: 26 - DNA, 500 bp, Homo sapiens
gggctgcctcttgttctcccgccgctgccgccgtctcctggtcgggtgccgcggccagaggcgcgcggggctgccgaggcacccgcactatgcaggcagactgccggccgccgcgatggcgagccgggcggtggtgagagccaggcgctgcccgcagtgtccccaagtccgggccgcggccgccgcccccgcctgggccgcgctccccctctcccgctccctccctccctgctccaactcctcctccttctccatgcctctgttcctcctgctcttacttgtcctgctcctgctgctcgaggacgctggagcccagcaaggtgagtggtcccaggggagtgagcggcgggggaacctccgtcggggctgcgggtctcttggcccctgaacaaatctgcagacgctgcagggaggcgcggtgcgctttcgtgcgtggccggacctggctgggtactcacggggttgagttgcaaaggcaaggtcttgcccgtctgtccccc

SEQ ID NO: 27 - DNA, 500 bp, Homo sapiens
aaaaaaaataaaatagaaagtatgggtggtataaaatatttgatgtcgtgcatatttttaaatcttcacaaaaaaatcattatagaggaagtaaaatatgtgagaataatttatcattcttttcactttttttaaaggtgatggatgtggacacactgtactaggccctgagagtggaacccttacatccataaactacccacagacctatcccaacagcactgtttgtgaatgggagatccgtgtaaagatgggagagagagttcgcatcaaatttggtgactttgacattgaagattctgattcttgtcactttaattacttgagaatttataatggaattggagtcagcagaactgaaataggtaggacttttttttgtaaatgtacatgtaaatacatcattgtctcaatgatgtatttttttgatattaacattaaataaccatgcagtgtaaggtgctatgtgggaaattattgaatatgtgtaaaaattttga

SEQ ID NO: 28 - DNA, 500 bp, Homo sapiens
ttacttgtcatttttttcctccagagcaatatgattggtaatcattaaacattttagtcattattccatactaatttaacagccagcttggtatattattctgcttctgttatctacattgcaaatattattgacttcatgagagtcatcccaaataatctgcttttctttcattcctaggcaaatactgtggtctggggttgcaaatgaaccattcaattgaatcaaaaggcaatgaaatcacattgctgttcatgagtggaatccatgtttctggacgcggatttttggcctcatactctgttatagataaacaaggtaattttcaccttttgcaatggttcctacagtttgtaatttctaacaacaaaaataaaatgctttttttaagtacctgttttctgaaattggaatagagcagaaagactgaacaattaatagcacatatataaatttgacaaataaggtgagttcagtttcaaagttccaaaaataaaagt

SEQ ID NO: 29 - DNA, 500 bp, Homo sapiens
gtagtaaacagtattgaggggacattgctccaaaaatcaagaaacttaatatcagaaagaaattgtgattaacaaaagttggtaaagtttttagtaccactttgacagtgccattgagatttttgaaaattagagtagtacagtttgggattctgagacacttggagtttcagcaaggtaatcctgtatattaatggaatcagttttttatttttgtttttctagatctaattacttgtttggacactgcatccaattttttggaacctgagttcaggtatgattatatttgtgactttttttttttttttgcttaaatgttacttttcattcacttcttcattttcattctgtatactcttagggttaatgtatattgtgtgatacatatcattgttccgagacttcaataaattagagcttacttaaagcataaagccatttagttgcatgtttacattttgcttttattgtagcatttagatctgcaaaatgaaagt

SEQ ID NO: 30 - DNA, 500 bp, Homo sapiens
ggttgcagtgagccaagaaggtgccattgcactccagcctgggcgacaagagtgaaactccgtgtcaaaaaaaagaagtggtgggtctttgagggtagtgaaattatggtattcaccttgaactgaaaaatgtgtctgtgatatttttggggggttgcttttttccttgtaaaatacagcattatgtaaccttttgcttgttttcatttctcagtaagtactgcccagctggttgtctgcttccttttgctgagatatctggaacaattcctcatggatatagagatgtaagccagatataaacttatttgaaaattgtgatataactctatttttttagaactccagagtagttagaggtgtgttaatcatatatcattatcttttgcctttttaagacattttttaatcatatgtgttttattctttttcttctgctaaaactgttgtgttacagtaccaataatctatttttgtttatgaagtttatgaagaaactg

SEQ ID NO: 31 - DNA, 500 bp, Homo sapiens
catatgtgttttattctttttcttctgctaaaactgttgtgttacagtaccaataatctatttttgtttatgaagtttatgaagaaactgcctagattaactgatttctcaggaagtagagtgtcttgtttatgaattattaagagaaaattaaaaatgaatcttttcttttcccttccttaagtcctcgccattgtgcatggctggtgtgcatgcaggagtagtgtcaaacacgttgggcggccaaatcagtgttgtaattagtaaaggtattccctattatgaaagttctttggctaacaacgtcacatctgtggtgtaagtatatatgcaatttaaaaaatatatgctatatataattagcattctgggtagcattgggctaagaactagctagatctcagcagcatcactgttaatggtgttcatgaaagaacatttaggcagtaagcactcacattagtgtgctcagtgtctgtttttttgttttgttttgtttg

SEQ ID NO: 32 - DNA, 500 bp, Homo sapiens
gggtgctagatacttgtagagaaagctgtttatgctgatttcagaagttgtcctgcttagaaatgagacttttttcctaggaacaatattagttgtctaatttttagccacacagtcttcttggttcttttattgggtggggggaaccaaatcaaaagaaaatgtttcactaaatatggggcatcatgtgttatgtgagtatcaccaaaaaaaacccttctctctttcaggggacacttatctacaagtctttttacatttaagacaagtggtaagccttttatggactttataattttttatttatataatattttttgaacactgttaggcactgtgtcagttgctgatttattgtactttctcacgtagttgtcttaataacactatgaggtaagatagttatctaataatataatgaatagagcaaattgaaagttttatatttatagagaaatggtcatgtaagcaaggaaggttccttaagaaagatgactttt

SEQ ID NO: 33 - DNA, 500 bp, Homo sapiens
tagttttgaaatttctctgttacagtgtattattagtgtagtgtatgttaattaaagatggaatagaaagttttcagtgagcaggtctacatgcattttgaaattatacctggaaaccaaatttagtcttttctttgtttcaggatgttatggaacactggggatggagtctggtgtgatcgcggatcctcaaataacagcatcatctgtgctggagtggactgaccacacagggcaagagaacagttggaaacccaaaaaagccaggctgaaaaaacctggaccgccttgggctgcttttgccactgatgaataccagtggttacaaatagatttgaataaggaaaagaaaataacaggttaagagacaaaaattttctaaagcaatttgtaacacttattttatttgaaaaactagtaacatctaagattttcagagaaaattaaggcattaaaattgtaattatatggttttatattttcttcactgatgttaatat

SEQ ID NO: 34 - DNA, 500 bp, Homo sapiens
cctctctctccaagggaataaggccaaggatttagtaattgttatgacaaaaacgtgcacttggatatggaaatgagccataattagaaatgttcgagctgtgcatttcaagtgtttatactttgatgctgaacaaacttagggaatcagaccatgtgcattaatgaaagcaactcttcattccccaggcattataaccactggatccaccatggtggagcacaattactatgtgtctgcctacagaatcctgtacagtgatgatgggcagaaatggactgtgtacagagagcctggtgtggagcaagataaggtaaaatgattaccaaggtatttttaaaaaacattttttaaatgggtggttgccaagtcacagagctctcctgtataaaagagatgtagaagtttagaatgtcttatactaccctcttgtggttattttaggtagtgttattggccttactctactgactttagaaaattctcagtagttctaggtt

SEQ ID NO: 35 - DNA, 500 bp, Homo sapiens
agcatcttaatatttttccaaattatgattattttcttaaaaattgatccttattgtcatactttccagatgtaatttaaggcctagagcaaagaacaaaaacatgtttaagtggagaaatttagccctgacctctattgatttgaagtaccaattttttgttgtttttttaaagatatttcaaggaaacaaagattatcaccaggatgtgcgtaataactttttgccaccaattattgcacgttttattagagtgaatcctacccaatggcagcagaaaattgccatgaaaatggagctgctcggatgtcagtttattcctaaaggtaaagagacagttcgtttaagatatgtttttactggattgccaatttaaaatggttttgctgttctcagagagagtaattaataatgtgagtgtctttctggttaaactctaattacaatatcttcttaaagggatgttaaaaaaaatggttaccctattaaaaagttagtta

SEQ ID NO: 36 - DNA, 500 bp, Homo sapiens
aattatttaaaaactgagaaatcctggcatatttgtgcctttattgcagagtataatatttatccttccaatatatttctctctgagagttaaaattattctgcagaattaaatactttaggaaatattgaagacatttagcattactatttttaattatgatttttaaaaacaaaatgtttaaatttttctaaaccatttttcaaggtcgtcctccaaaacttactcaacctccacctcctcggaacagcaatgacctcaaaaacactacagcccctccaaaaatagccaaaggtatgcctgtttttaatgtgagaatttgatattccttggatttatggtgcaactttccaaaaatatattaaaagcattatcaaataagtttatgttaatcaagttggtacagattaaaaagtaaagagcttttgaaacttttttaaaggtcgtgccccaaaatttacgcaaccactacaacctcgcagtagcaatgaatttcctgc

SEQ ID NO: 37 - DNA, 500 bp, Homo sapiens
gacctcaaaaacactacagcccctccaaaaatagccaaaggtatgcctgtttttaatgtgagaatttgatattccttggatttatggtgcaactttccaaaaatatattaaaagcattatcaaataagtttatgttaatcaagttggtacagattaaaaagtaaagagcttttgaaacttttttaaaggtcgtgccccaaaatttacgcaaccactacaacctcgcagtagcaatgaatttcctgcacagacagaacaaacaactgccagtcctgatatcagaaatactaccgtaactccaaatgtaaccaaaggtatgcaaacatggaaatgaacgcctagtacatagtattgttttgttcttaactgaagttttcacgggcatttttaaaccttcagtttattttcttccaactatagtttttaagtatagcaaaaaaaaaaatttattttctttcataggaaaagactgcattgatctaagttgtagtctctgctgc

SEQ ID NO: 38 - DNA, 500 bp, Homo sapiens
ggaatgaatagtgtaacaagcttttgtgtatctgcaaaagcttctacagttaccaccacatgcccagtcttgacaaataatgtttatggtatttccgtgaggtagaaaaatgttgaaagatgatgtggttaattttccctagtgatttctgcctttggtgttaatatggcttttgtttgtctgtttttgtctttgtaacttcagatgtagcgctggctgcagttcttgtccctgtgctggtcatggtcctcactactctcattctcatattagtgtgtgcttggcactggagaaacaggttagtacataactagttcacctgagtccaaaactaccaaatgtgaagtagaagctaaatatagaagatgaaaatgtttacctgtttgagagtgagagttaaggtaattattaaaatgaaaatttcatgcttctcctttattcccattaaaaataaataagttcaattccacaatcagttactaagtaccttttttgtatca

SEQ ID NO: 39 - DNA, 500 bp, Homo sapiens
ctttgtagtgacatggatgaagctggaaaccatcattctcagcaaactgtcccaaggacaaaaaaccaaacaccgcatgttctcactcataggtgggaattgaacaatgagaacacttggacacaggaaggggaatatcacataccaaggcctgtcatggggtagggggagcaggcttaccagtgaatttactctgtttgctaacttctcttctgtatttttccagaaagaaaaaaactgaaggcacctatgacttaccttactgggaccgggcaggtaactcacgtggtctttgcatctcatttctatcagagggatgtcgctcccctacagggggcagtagtgaaaaaagagtcattctctggcccaggtgaactccccgacactgttagaacaatggcattactcttcagttctcaccatttttacccttctgcaaagtctcttgtaattcctaagtaatgaaatgaaaagtacaaatttcttaaaacaagctctgt

SEQ ID NO: 40 - DNA, 500 bp, Homo sapiens
ctgcttggtgctggatagtctatcacagcaaaaaaaaaaaaaaaagttggattataaaggcaagtttgtagagatatttgttatagaaatggcttaaatgcaacaggatttttctcttgatactactgtttgagatacaggtttttatttaatggtctctttggcttgccgtcacaatgaaggttggtggaaaggaatgaagcagtttcttcctgcaaaagcagtggaccatgaggaaaccccagttcgctatagcagcagcgaagttaatcacctgagtccaagagaagtcaccacagtgctgcaggctgactctgcaggtaactatgttgcagccttctggtaccaggcagagggagaaactgcttaggcttgctataaagtgctttgggatttacagttctttgatccctttcatgtttaagaaatgaattgtttcctaaaggtagaaccacttttttaaaaggtgactcttcagagagtcatttctgtatcttgga

SEQ ID NO: 41 - DNA, 500 bp, Homo sapiens
atccacagagtatgctcagccactggtaggaggaattgttggtacacttcatcaaagatctacctttaaaccagaagaaggaaaagaagcaggctatgcagacctagatccttacaactcaccagggcaggaagtttatcatgcctatgctgaaccactcccaattacggggcctgagtatgcaaccccaatcatcatggacatgtcagggcaccccacaacttcagttggtcagccctccacatccactttcaaggctacggggaaccaacctcccccactagtgggaacttacaatacacttctctccaggactgacagctgctcctcagcccaggcccagtatgataccccgaaagctgggaagccaggtctacctgccccagacgaattggtgtaccaggtgccacagagcacacaagaagtatcaggagcaggaagggatggggaatgtgatgtttttaaagaaatcctttgaagatgatgctgctttttacaaa

SEQ ID NO: 42 - DNA, 1000 bp, Homo sapiens
tgttgcaacaactggcggcagttttaacaaaacaagccacgctctgccattaaaaaaaaaaaaaaagtcccttagactcagctcccagctaaaacaacccaggatccaggtttcccacccccttccagtggtctgatgtgcgtcggaggagcgaggagaaaggagcaaggagcaagctgggcctgaggagcagggagcgcgggatgggggacgtcccggaagtcactgacgagctggctgccgcctctgcgggatcagtttgggctgagggcccaacaataacaggcgcccgggccgggcgggggccgcggggatccgaagctggagggcgggacctggattggaggaggcgggtgggacgcctccaaaggccgaaggggattggaagagggaggtaaggggcggtggagctcgaggaatgggccggagaactagcgggtcgagcgaggagcgagcatgctgattgggtttcgcggaaggagacggaccagagctggccgaggattggccgcggtgcctagggggcgtggccggcgcgcgcgagagggagcgcgagggagctggctgcgggcctgcctgccagctagccggagccgcgggtgagcgcggcgagcggcgaccctggtgaggagcgcggcgcgggaggcacgttccttagctccgccgcggccgtcctccgcggctcgaggactccgcttccttccctcccctcccctgcgctccggcctggggtctcggcgcggggagcggagggaagggacgaaggaggagtaggtgaaagcggggtgaggggcggaagggtcccggcgcggggtgaggcgagggctgcctcttgttctcccgccgctgccgccgtctcctggtcgggtgccgcggccagaggcgcgcggggctgccgaggcacccgcactatgcaggcagactgccggccgccgcgatggcgagccgggcggtggtgagagccaggcgctgcccgcagtgtccccaagtccgggccgcg

SEQ ID NO: 43 - DNA, 17 bp, Homo sapiens
aaacagaaagaaaaaaa

SEQ ID NO: 44 - DNA, 17 bp, Homo sapiens
aacagaaagaaaaaaac

SEQ ID NO: 45 - DNA, 17 bp, Homo sapiens
acagaaagaaaaaaact

SEQ ID NO: 46 - DNA, 17 bp, Homo sapiens
cagaaagaaaaaaactg

SEQ ID NO: 47 - DNA, 17 bp, Homo sapiens
agaaagaaaaaaactga

SEQ ID NO: 48 - DNA, 17 bp, Homo sapiens
gaaagaaaaaaactgaa

SEQ ID NO: 49 - DNA, 17 bp, Homo sapiens
aaagaaaaaaactgaag

SEQ ID NO: 50 - DNA, 17 bp, Homo sapiens
aagaaaaaaactgaagg

SEQ ID NO: 51 - DNA, 17 bp, Homo sapiens
agaaaaaaactgaaggc

SEQ ID NO: 52 - DNA, 17 bp, Homo sapiens
gaaaaaaactgaaggca

SEQ ID NO: 53 - DNA, 17 bp, Homo sapiens
aaaaaaactgaaggcac

SEQ ID NO: 54 - DNA, 17 bp, Homo sapiens
aaaaaactgaaggcacc

SEQ ID NO: 55 - DNA, 17 bp, Homo sapiens
aaaaactgaaggcacct

SEQ ID NO: 56 - DNA, 17 bp, Homo sapiens
aaaactgaaggcaccta

SEQ ID NO: 57 - DNA, 17 bp, Homo sapiens
aaactgaaggcacctat

SEQ ID NO: 58 - DNA, 17 bp, Homo sapiens
aactgaaggcacctatg

SEQ ID NO: 59 - DNA, 17 bp, Homo sapiens
actgaaggcacctatga

SEQ ID NO: 60 - DNA, 17 bp, Homo sapiens
ctgaaggcacctatgac

SEQ ID NO: 61 - DNA, 17 bp, Homo sapiens
tgaaggcacctatgact

SEQ ID NO: 62 - DNA, 17 bp, Homo sapiens
gaaggcacctatgactt

SEQ ID NO: 63 - DNA, 17 bp, Homo sapiens
aaggcacctatgactta

SEQ ID NO: 64 - DNA, 17 bp, Homo sapiens
aggcacctatgacttac

SEQ ID NO: 65 - DNA, 17 bp, Homo sapiens
ggcacctatgacttacc

SEQ ID NO: 66 - DNA, 17 bp, Homo sapiens
gcacctatgacttacct

SEQ ID NO: 67 - DNA, 17 bp, Homo sapiens
cacctatgacttacctt

SEQ ID NO: 68 - DNA, 17 bp, Homo sapiens
acctatgacttacctta

SEQ ID NO: 69 - DNA, 17 bp, Homo sapiens
cctatgacttaccttac

SEQ ID NO: 70 - DNA, 17 bp, Homo sapiens
ctatgacttaccttact

SEQ ID NO: 71 - DNA, 17 bp, Homo sapiens
tatgacttaccttactg

SEQ ID NO: 72 - DNA, 17 bp, Homo sapiens
atgacttaccttactgg

SEQ ID NO: 73 - DNA, 17 bp, Homo sapiens
tgacttaccttactggg

SEQ ID NO: 74 - DNA, 17 bp, Homo sapiens
gacttaccttactggga

SEQ ID NO: 75 - DNA, 17 bp, Homo sapiens
acttaccttactgggac

SEQ ID NO: 76 - DNA, 17 bp, Homo sapiens
cttaccttactgggacc

SEQ ID NO: 77 - DNA, 17 bp, Homo sapiens
ttaccttactgggaccg

SEQ ID NO: 78 - DNA, 17 bp, Homo sapiens
taccttactgggaccgg

SEQ ID NO: 79 - DNA, 17 bp, Homo sapiens
accttactgggaccggg

SEQ ID NO: 80 - DNA, 17 bp, Homo sapiens
ccttactgggaccgggc

SEQ ID NO: 81 - DNA, 17 bp, Homo sapiens
cttactgggaccgggca

SEQ ID NO: 82 - DNA, 17 bp, Homo sapiens
ttactgggaccgggcag

SEQ ID NO: 83 - DNA, 17 bp, Homo sapiens
tactgggaccgggcagg

SEQ ID NO: 84 - DNA, 17 bp, Homo sapiens
actgggaccgggcaggt

SEQ ID NO: 85 - DNA, 17 bp, Homo sapiens
ctgggaccgggcaggtt

SEQ ID NO: 86 - DNA, 17 bp, Homo sapiens
tgggaccgggcaggttg

SEQ ID NO: 87 - DNA, 17 bp, Homo sapiens
gggaccgggcaggttgg

SEQ ID NO: 88 - DNA, 17 bp, Homo sapiens
ggaccgggcaggttggt

SEQ ID NO: 89 - DNA, 17 bp, Homo sapiens
gaccgggcaggttggtg

SEQ ID NO: 90 - DNA, 17 bp, Homo sapiens
accgggcaggttggtgg

SEQ ID NO: 91 - DNA, 17 bp, Homo sapiens
ccgggcaggttggtgga

SEQ ID NO: 92 - DNA, 17 bp, Homo sapiens
cgggcaggttggtggaa

SEQ ID NO: 93 - DNA, 17 bp, Homo sapiens
gggcaggttggtggaaa

SEQ ID NO: 94 - DNA, 17 bp, Homo sapiens
ggcaggttggtggaaag

SEQ ID NO: 95 - DNA, 17 bp, Homo sapiens
gcaggttggtggaaagg

SEQ ID NO: 96 - DNA, 17 bp, Homo sapiens
caggttggtggaaagga

SEQ ID NO: 97 - DNA, 17 bp, Homo sapiens
aggttggtggaaaggaa

SEQ ID NO: 98 - DNA, 17 bp, Homo sapiens
ggttggtggaaaggaat

SEQ ID NO: 99 - DNA, 17 bp, Homo sapiens
gttggtggaaaggaatg

SEQ ID NO: 100 - DNA, 17 bp, Homo sapiens
ttggtggaaaggaatga

SEQ ID NO: 101 - DNA, 17 bp, Homo sapiens
tggtggaaaggaatgaa

SEQ ID NO: 102 - DNA, 17 bp, Homo sapiens
ggtggaaaggaatgaag

SEQ ID NO: 103 - DNA, 17 bp, Homo sapiens
gtggaaaggaatgaagc

SEQ ID NO: 104 - DNA, 17 bp, Homo sapiens
tggaaaggaatgaagca

SEQ ID NO: 105 - DNA, 17 bp, Homo sapiens
ggaaaggaatgaagcag

SEQ ID NO: 106 - DNA, 17 bp, Homo sapiens
gaaaggaatgaagcagt

SEQ ID NO: 107 - DNA, 17 bp, Homo sapiens
aaaggaatgaagcagtt

SEQ ID NO: 108 - DNA, 17 bp, Homo sapiens
aaggaatgaagcagttt

SEQ ID NO: 109 - DNA, 17 bp, Homo sapiens
aggaatgaagcagtttc

SEQ ID NO: 110 - DNA, 17 bp, Homo sapiens
ggaatgaagcagtttct

SEQ ID NO: 111 - DNA, 17 bp, Homo sapiens
gaatgaagcagtttctt

SEQ ID NO: 112 - DNA, 17 bp, Homo sapiens
aatgaagcagtttcttc

SEQ ID NO: 113 - DNA, 17 bp, Homo sapiens
atgaagcagtttcttcc

SEQ ID NO: 114 - DNA, 17 bp, Homo sapiens
tgaagcagtttcttcct

SEQ ID NO: 115 - DNA, 17 bp, Homo sapiens
gaagcagtttcttcctg

SEQ ID NO: 116 - DNA, 17 bp, Homo sapiens
aagcagtttcttcctgc

SEQ ID NO: 117 - DNA, 17 bp, Homo sapiens
agcagtttcttcctgca

SEQ ID NO: 118 - DNA, 17 bp, Homo sapiens
gcagtttcttcctgcaa

SEQ ID NO: 119 - DNA, 17 bp, Homo sapiens
cagtttcttcctgcaaa

SEQ ID NO: 120 - DNA, 17 bp, Homo sapiens
agtttcttcctgcaaaa

SEQ ID NO: 121 - DNA, 17 bp, Homo sapiens
gtttcttcctgcaaaag

SEQ ID NO: 122 - DNA, 17 bp, Homo sapiens
tttcttcctgcaaaagc

SEQ ID NO: 123 - DNA, 17 bp, Homo sapiens
ttcttcctgcaaaagca

SEQ ID NO: 124 - DNA, 17 bp, Homo sapiens
tcttcctgcaaaagcag

SEQ ID NO: 125 - DNA, 17 bp, Homo sapiens
cttcctgcaaaagcagt

SEQ ID NO: 126 - DNA, 17 bp, Homo sapiens
ttcctgcaaaagcagtg

SEQ ID NO: 127 - DNA, 17 bp, Homo sapiens
tcctgcaaaagcagtgg

SEQ ID NO: 128 - DNA, 17 bp, Homo sapiens
cctgcaaaagcagtgga

SEQ ID NO: 129 - DNA, 17 bp, Homo sapiens
ctgcaaaagcagtggac

SEQ ID NO: 130 - DNA, 17 bp, Homo sapiens
tgcaaaagcagtggacc

SEQ ID NO: 131 - DNA, 17 bp, Homo sapiens
gcaaaagcagtggacca

SEQ ID NO: 132 - DNA, 17 bp, Homo sapiens
caaaagcagtggaccat

SEQ ID NO: 133 - DNA, 17 bp, Homo sapiens
aaaagcagtggaccatg

SEQ ID NO: 134 - DNA, 17 bp, Homo sapiens
aaagcagtggaccatga

SEQ ID NO: 135 - DNA, 17 bp, Homo sapiens
aagcagtggaccatgag

SEQ ID NO: 136 - DNA, 17 bp, Homo sapiens
agcagtggaccatgagg

SEQ ID NO: 137 - DNA, 17 bp, Homo sapiens
gcagtggaccatgagga

SEQ ID NO: 138 - DNA, 17 bp, Homo sapiens
cagtggaccatgaggaa

SEQ ID NO: 139 - DNA, 17 bp, Homo sapiens
agtggaccatgaggaaa

SEQ ID NO: 140 - DNA, 17 bp, Homo sapiens
gtggaccatgaggaaac

SEQ ID NO: 141 - DNA, 17 bp, Homo sapiens
tggaccatgaggaaacc

SEQ ID NO: 142 - DNA, 17 bp, Homo sapiens
ggaccatgaggaaaccc

SEQ ID NO: 143 - DNA, 17 bp, Homo sapiens
gaccatgaggaaacccc

SEQ ID NO: 144 - DNA, 17 bp, Homo sapiens
accatgaggaaacccca

SEQ ID NO: 145 - DNA, 17 bp, Homo sapiens
ccatgaggaaaccccag

SEQ ID NO: 146 - DNA, 17 bp, Homo sapiens
catgaggaaaccccagt

SEQ ID NO: 147 - DNA, 17 bp, Homo sapiens
atgaggaaaccccagtt

SEQ ID NO: 148 - DNA, 17 bp, Homo sapiens
tgaggaaaccccagttc

SEQ ID NO: 149 - DNA, 17 bp, Homo sapiens
gaggaaaccccagttcg

SEQ ID NO: 150 - DNA, 17 bp, Homo sapiens
aggaaaccccagttcgc

SEQ ID NO: 151 - DNA, 17 bp, Homo sapiens
ggaaaccccagttcgct

SEQ ID NO: 152 - DNA, 17 bp, Homo sapiens
gaaaccccagttcgcta

SEQ ID NO: 153 - DNA, 17 bp, Homo sapiens
aaaccccagttcgctat

SEQ ID NO: 154 - DNA, 17 bp, Homo sapiens
aaccccagttcgctata

SEQ ID NO: 155 - DNA, 17 bp, Homo sapiens
accccagttcgctatag

SEQ ID NO: 156 - DNA, 17 bp, Homo sapiens
ccccagttcgctatagc

SEQ ID NO: 157 - DNA, 17 bp, Homo sapiens
cccagttcgctatagca

SEQ ID NO: 158 - DNA, 17 bp, Homo sapiens
ccagttcgctatagcag

SEQ ID NO: 159 - DNA, 17 bp, Homo sapiens
cagttcgctatagcagc

SEQ ID NO: 160 - DNA, 17 bp, Homo sapiens
agttcgctatagcagca

SEQ ID NO: 161 - DNA, 17 bp, Homo sapiens
gttcgctatagcagcag

SEQ ID NO: 162 - DNA, 17 bp, Homo sapiens
ttcgctatagcagcagc

SEQ ID NO: 163 - DNA, 17 bp, Homo sapiens
tcgctatagcagcagcg

SEQ ID NO: 164 - DNA, 17 bp, Homo sapiens
cgctatagcagcagcga

SEQ ID NO: 165 - DNA, 17 bp, Homo sapiens
gctatagcagcagcgaa

SEQ ID NO: 166 - DNA, 17 bp, Homo sapiens
ctatagcagcagcgaag

SEQ ID NO: 167 - DNA, 17 bp, Homo sapiens
tatagcagcagcgaagt

SEQ ID NO: 168 - DNA, 17 bp, Homo sapiens
atagcagcagcgaagtt

SEQ ID NO: 169 - DNA, 17 bp, Homo sapiens
tagcagcagcgaagtta

SEQ ID NO: 170 - DNA, 17 bp, Homo sapiens
agcagcagcgaagttaa

SEQ ID NO: 171 - DNA, 17 bp, Homo sapiens
gcagcagcgaagttaat

SEQ ID NO: 172 - DNA, 17 bp, Homo sapiens
cagcagcgaagttaatc

SEQ ID NO: 173 - DNA, 17 bp, Homo sapiens
agcagcgaagttaatca

SEQ ID NO: 174 - DNA, 17 bp, Homo sapiens
gcagcgaagttaatcac

SEQ ID NO: 175 - DNA, 17 bp, Homo sapiens
cagcgaagttaatcacc

SEQ ID NO: 176 - DNA, 17 bp, Homo sapiens
agcgaagttaatcacct

SEQ ID NO: 177 - DNA, 17 bp, Homo sapiens
gcgaagttaatcacctg

SEQ ID NO: 178 - DNA, 17 bp, Homo sapiens
cgaagttaatcacctga

SEQ ID NO: 179 - DNA, 17 bp, Homo sapiens
gaagttaatcacctgag

SEQ ID NO: 180 - DNA, 17 bp, Homo sapiens
aagttaatcacctgagt

SEQ ID NO: 181 - DNA, 17 bp, Homo sapiens
agttaatcacctgagtc

SEQ ID NO: 182 - DNA, 17 bp, Homo sapiens
gttaatcacctgagtcc

SEQ ID NO: 183 - DNA, 17 bp, Homo sapiens
ttaatcacctgagtcca

SEQ ID NO: 184 - DNA, 17 bp, Homo sapiens
taatcacctgagtccaa

SEQ ID NO: 185 - DNA, 17 bp, Homo sapiens
aatcacctgagtccaag

SEQ ID NO: 186 - DNA, 17 bp, Homo sapiens
atcacctgagtccaaga

SEQ ID NO: 187 - DNA, 17 bp, Homo sapiens
tcacctgagtccaagag

SEQ ID NO: 188 - DNA, 17 bp, Homo sapiens
cacctgagtccaagaga

SEQ ID NO: 189 - DNA, 17 bp, Homo sapiens
acctgagtccaagagaa

SEQ ID NO: 190 - DNA, 17 bp, Homo sapiens
cctgagtccaagagaag

SEQ ID NO: 191 - DNA, 17 bp, Homo sapiens
ctgagtccaagagaagt

SEQ ID NO: 192 - DNA, 17 bp, Homo sapiens
tgagtccaagagaagtc

SEQ ID NO: 193 - DNA, 17 bp, Homo sapiens
gagtccaagagaagtca

SEQ ID NO: 194 - DNA, 17 bp, Homo sapiens
agtccaagagaagtcac

SEQ ID NO: 195 - DNA, 17 bp, Homo sapiens
gtccaagagaagtcacc

SEQ ID NO: 196 - DNA, 17 bp, Homo sapiens
tccaagagaagtcacca

SEQ ID NO: 197 - DNA, 17 bp, Homo sapiens
ccaagagaagtcaccac

SEQ ID NO: 198 - DNA, 17 bp, Homo sapiens
caagagaagtcaccaca

SEQ ID NO: 199 - DNA, 17 bp, Homo sapiens
aagagaagtcaccacag

SEQ ID NO: 200 - DNA, 17 bp, Homo sapiens
agagaagtcaccacagt

SEQ ID NO: 201 - DNA, 17 bp, Homo sapiens
gagaagtcaccacagtg

SEQ ID NO: 202 - DNA, 17 bp, Homo sapiens
agaagtcaccacagtgc

SEQ ID NO: 203 - DNA, 17 bp, Homo sapiens
gaagtcaccacagtgct

SEQ ID NO: 204 - DNA, 17 bp, Homo sapiens
aagtcaccacagtgctg

SEQ ID NO: 205 - DNA, 17 bp, Homo sapiens
agtcaccacagtgctgc

SEQ ID NO: 206 - DNA, 17 bp, Homo sapiens
gtcaccacagtgctgca

SEQ ID NO: 207 - DNA, 17 bp, Homo sapiens
tcaccacagtgctgcag

SEQ ID NO: 208 - DNA, 17 bp, Homo sapiens
caccacagtgctgcagg

SEQ ID NO: 209 - DNA, 17 bp, Homo sapiens
accacagtgctgcaggc

SEQ ID NO: 210 - DNA, 17 bp, Homo sapiens
ccacagtgctgcaggct

SEQ ID NO: 211 - DNA, 17 bp, Homo sapiens
cacagtgctgcaggctg

SEQ ID NO: 212 - DNA, 17 bp, Homo sapiens
acagtgctgcaggctga

SEQ ID NO: 213 - DNA, 17 bp, Homo sapiens
cagtgctgcaggctgac

SEQ ID NO: 214 - DNA, 17 bp, Homo sapiens
agtgctgcaggctgact

SEQ ID NO: 215 - DNA, 17 bp, Homo sapiens
gtgctgcaggctgactc

SEQ ID NO: 216 - DNA, 17 bp, Homo sapiens
tgctgcaggctgactct

SEQ ID NO: 217 - DNA, 17 bp, Homo sapiens
gctgcaggctgactctg

SEQ ID NO: 218 - DNA, 17 bp, Homo sapiens
ctgcaggctgactctgc

SEQ ID NO: 219 - DNA, 17 bp, Homo sapiens
tgcaggctgactctgca

SEQ ID NO: 220 - DNA, 17 bp, Homo sapiens
gcaggctgactctgcag

SEQ ID NO: 221 - DNA, 17 bp, Homo sapiens
caggctgactctgcaga

SEQ ID NO: 222 - DNA, 17 bp, Homo sapiens
aggctgactctgcagag

SEQ ID NO: 223 - DNA, 17 bp, Homo sapiens
ggctgactctgcagagt

SEQ ID NO: 224 - DNA, 17 bp, Homo sapiens
gctgactctgcagagta

SEQ ID NO: 225 - DNA, 17 bp, Homo sapiens
ctgactctgcagagtat

SEQ ID NO: 226 - DNA, 17 bp, Homo sapiens
tgactctgcagagtatg

SEQ ID NO: 227 - DNA, 17 bp, Homo sapiens
gactctgcagagtatgc

SEQ ID NO: 228 - DNA, 17 bp, Homo sapiens
actctgcagagtatgct

SEQ ID NO: 229 - DNA, 17 bp, Homo sapiens
ctctgcagagtatgctc

SEQ ID NO: 230 - DNA, 17 bp, Homo sapiens
tctgcagagtatgctca

SEQ ID NO: 231 - DNA, 17 bp, Homo sapiens
ctgcagagtatgctcag

SEQ ID NO: 232 - DNA, 17 bp, Homo sapiens
tgcagagtatgctcagc

SEQ ID NO: 233 - DNA, 17 bp, Homo sapiens
gcagagtatgctcagcc

SEQ ID NO: 234 - DNA, 17 bp, Homo sapiens
cagagtatgctcagcca

SEQ ID NO: 235 - DNA, 17 bp, Homo sapiens
agagtatgctcagccac

SEQ ID NO: 236 - DNA, 17 bp, Homo sapiens
gagtatgctcagccact

SEQ ID NO: 237 - DNA, 17 bp, Homo sapiens
agtatgctcagccactg

SEQ ID NO: 238 - DNA, 17 bp, Homo sapiens
gtatgctcagccactgg

SEQ ID NO: 239 - DNA, 17 bp, Homo sapiens
tatgctcagccactggt

SEQ ID NO: 240 - DNA, 17 bp, Homo sapiens
atgctcagccactggta

SEQ ID NO: 241 - DNA, 17 bp, Homo sapiens
tgctcagccactggtag

SEQ ID NO: 242 - DNA, 17 bp, Homo sapiens
gctcagccactggtagg

SEQ ID NO: 243 - DNA, 17 bp, Homo sapiens
ctcagccactggtagga

SEQ ID NO: 244 - DNA, 17 bp, Homo sapiens
tcagccactggtaggag

SEQ ID NO: 245 - DNA, 17 bp, Homo sapiens
cagccactggtaggagg

SEQ ID NO: 246 - DNA, 17 bp, Homo sapiens
agccactggtaggagga

SEQ ID NO: 247 - DNA, 17 bp, Homo sapiens
gccactggtaggaggaa

SEQ ID NO: 248 - DNA, 17 bp, Homo sapiens
ccactggtaggaggaat

SEQ ID NO: 249 - DNA, 17 bp, Homo sapiens
cactggtaggaggaatt

SEQ ID NO: 250 - DNA, 17 bp, Homo sapiens
actggtaggaggaattg

SEQ ID NO: 251 - DNA, 17 bp, Homo sapiens
ctggtaggaggaattgt

SEQ ID NO: 252 - DNA, 17 bp, Homo sapiens
tggtaggaggaattgtt

SEQ ID NO: 253 - DNA, 17 bp, Homo sapiens
ggtaggaggaattgttg

SEQ ID NO: 254 - DNA, 17 bp, Homo sapiens
gtaggaggaattgttgg

SEQ ID NO: 255 - DNA, 17 bp, Homo sapiens
taggaggaattgttggt

SEQ ID NO: 256 - DNA, 17 bp, Homo sapiens
aggaggaattgttggta

SEQ ID NO: 257 - DNA, 17 bp, Homo sapiens
ggaggaattgttggtac

SEQ ID NO: 258 - DNA, 17 bp, Homo sapiens
gaggaattgttggtaca

SEQ ID NO: 259 - DNA, 17 bp, Homo sapiens
aggaattgttggtacac

SEQ ID NO: 260 - DNA, 17 bp, Homo sapiens
ggaattgttggtacact

SEQ ID NO: 261 - DNA, 17 bp, Homo sapiens
gaattgttggtacactt

SEQ ID NO: 262 - DNA, 17 bp, Homo sapiens
aattgttggtacacttc

SEQ ID NO: 263 - DNA, 17 bp, Homo sapiens
attgttggtacacttca

SEQ ID NO: 264 - DNA, 17 bp, Homo sapiens
ttgttggtacacttcat

SEQ ID NO: 265 - DNA, 17 bp, Homo sapiens
tgttggtacacttcatc

SEQ ID NO: 266 - DNA, 17 bp, Homo sapiens
gttggtacacttcatca

SEQ ID NO: 267 - DNA, 17 bp, Homo sapiens
ttggtacacttcatcaa

SEQ ID NO: 268 - DNA, 17 bp, Homo sapiens
tggtacacttcatcaaa

SEQ ID NO: 269 - DNA, 17 bp, Homo sapiens
ggtacacttcatcaaag

SEQ ID NO: 270 - DNA, 17 bp, Homo sapiens
gtacacttcatcaaaga

SEQ ID NO: 271 - DNA, 17 bp, Homo sapiens
tacacttcatcaaagat

SEQ ID NO: 272 - DNA, 17 bp, Homo sapiens
acacttcatcaaagatc

SEQ ID NO: 273 - DNA, 17 bp, Homo sapiens
cacttcatcaaagatct

SEQ ID NO: 274 - DNA, 17 bp, Homo sapiens
acttcatcaaagatcta

SEQ ID NO: 275 - DNA, 17 bp, Homo sapiens
cttcatcaaagatctac

SEQ ID NO: 276 - DNA, 17 bp, Homo sapiens
ttcatcaaagatctacc

SEQ ID NO: 277 - DNA, 17 bp, Homo sapiens
tcatcaaagatctacct

SEQ ID NO: 278 - DNA, 17 bp, Homo sapiens
catcaaagatctacctt

SEQ ID NO: 279 - DNA, 17 bp, Homo sapiens
atcaaagatctaccttt

SEQ ID NO: 280 - DNA, 17 bp, Homo sapiens
tcaaagatctaccttta

SEQ ID NO: 281 - DNA, 17 bp, Homo sapiens
caaagatctacctttaa

SEQ ID NO: 282 - DNA, 17 bp, Homo sapiens
aaagatctacctttaaa

SEQ ID NO: 283 - DNA, 17 bp, Homo sapiens
aagatctacctttaaac

SEQ ID NO: 284 - DNA, 17 bp, Homo sapiens
agatctacctttaaacc

SEQ ID NO: 285 - DNA, 17 bp, Homo sapiens
gatctacctttaaacca

SEQ ID NO: 286 - DNA, 17 bp, Homo sapiens
atctacctttaaaccag

SEQ ID NO: 287 - DNA, 17 bp, Homo sapiens
tctacctttaaaccaga

SEQ ID NO: 288 - DNA, 17 bp, Homo sapiens
ctacctttaaaccagaa

SEQ ID NO: 289 - DNA, 17 bp, Homo sapiens
tacctttaaaccagaag

SEQ ID NO: 290 - DNA, 17 bp, Homo sapiens
acctttaaaccagaaga

SEQ ID NO: 291 - DNA, 17 bp, Homo sapiens
cctttaaaccagaagaa

SEQ ID NO: 292 - DNA, 17 bp, Homo sapiens
ctttaaaccagaagaag

SEQ ID NO: 293 - DNA, 17 bp, Homo sapiens
tttaaaccagaagaagg

SEQ ID NO: 294 - DNA, 17 bp, Homo sapiens
ttaaaccagaagaagga

SEQ ID NO: 295 - DNA, 17 bp, Homo sapiens
taaaccagaagaaggaa

SEQ ID NO: 296 - DNA, 17 bp, Homo sapiens
aaaccagaagaaggaaa

SEQ ID NO: 297 - DNA, 17 bp, Homo sapiens
aaccagaagaaggaaaa

SEQ ID NO: 298 - DNA, 17 bp, Homo sapiens
accagaagaaggaaaag

SEQ ID NO: 299 - DNA, 17 bp, Homo sapiens
ccagaagaaggaaaaga

SEQ ID NO: 300 - DNA, 17 bp, Homo sapiens
cagaagaaggaaaagaa

SEQ ID NO: 301 - DNA, 17 bp, Homo sapiens
agaagaaggaaaagaag

SEQ ID NO: 302 - DNA, 17 bp, Homo sapiens
gaagaaggaaaagaagc

SEQ ID NO: 303 - DNA, 17 bp, Homo sapiens
aagaaggaaaagaagca

SEQ ID NO: 304 - DNA, 17 bp, Homo sapiens
agaaggaaaagaagcag

SEQ ID NO: 305 - DNA, 17 bp, Homo sapiens
gaaggaaaagaagcagg

SEQ ID NO: 306 - DNA, 17 bp, Homo sapiens
aaggaaaagaagcaggc

SEQ ID NO: 307 - DNA, 17 bp, Homo sapiens
aggaaaagaagcaggct

SEQ ID NO: 308 - DNA, 17 bp, Homo sapiens
ggaaaagaagcaggcta

SEQ ID NO: 309 - DNA, 17 bp, Homo sapiens
gaaaagaagcaggctat

SEQ ID NO: 310 - DNA, 17 bp, Homo sapiens
aaaagaagcaggctatg

SEQ ID NO: 311 - DNA, 17 bp, Homo sapiens
aaagaagcaggctatgc

SEQ ID NO: 312 - DNA, 17 bp, Homo sapiens
aagaagcaggctatgca

SEQ ID NO: 313 - DNA, 17 bp, Homo sapiens
agaagcaggctatgcag

SEQ ID NO: 314 - DNA, 17 bp, Homo sapiens
gaagcaggctatgcaga

SEQ ID NO: 315 - DNA, 17 bp, Homo sapiens
aagcaggctatgcagac

SEQ ID NO: 316 - DNA, 17 bp, Homo sapiens
agcaggctatgcagacc

SEQ ID NO: 317 - DNA, 17 bp, Homo sapiens
gcaggctatgcagacct

SEQ ID NO: 318 - DNA, 17 bp, Homo sapiens
caggctatgcagaccta

SEQ ID NO: 319 - DNA, 17 bp, Homo sapiens
aggctatgcagacctag

SEQ ID NO: 320 - DNA, 17 bp, Homo sapiens
ggctatgcagacctaga

SEQ ID NO: 321 - DNA, 17 bp, Homo sapiens
gctatgcagacctagat

SEQ ID NO: 322 - DNA, 17 bp, Homo sapiens
ctatgcagacctagatc

SEQ ID NO: 323 - DNA, 17 bp, Homo sapiens
tatgcagacctagatcc

SEQ ID NO: 324 - DNA, 17 bp, Homo sapiens
atgcagacctagatcct

SEQ ID NO: 325 - DNA, 17 bp, Homo sapiens
tgcagacctagatcctt

SEQ ID NO: 326 - DNA, 17 bp, Homo sapiens
gcagacctagatcctta

SEQ ID NO: 327 - DNA, 25 bp, Homo sapiens
aaacagaaagaaaaaaactgaaggc

SEQ ID NO: 328 - DNA, 25 bp, Homo sapiens
aacagaaagaaaaaaactgaaggca

SEQ ID NO: 329 - DNA, 25 bp, Homo sapiens
acagaaagaaaaaaactgaaggcac

SEQ ID NO: 330 - DNA, 25 bp, Homo sapiens
cagaaagaaaaaaactgaaggcacc

SEQ ID NO: 331 - DNA, 25 bp, Homo sapiens
agaaagaaaaaaactgaaggcacct

SEQ ID NO: 332 - DNA, 25 bp, Homo sapiens
gaaagaaaaaaactgaaggcaccta

SEQ ID NO: 333 - DNA, 25 bp, Homo sapiens
aaagaaaaaaactgaaggcacctat

SEQ ID NO: 334 - DNA, 25 bp, Homo sapiens
aagaaaaaaactgaaggcacctatg

SEQ ID NO: 335 - DNA, 25 bp, Homo sapiens
agaaaaaaactgaaggcacctatga

SEQ ID NO: 336 - DNA, 25 bp, Homo sapiens
gaaaaaaactgaaggcacctatgac

SEQ ID NO: 337 - DNA, 25 bp, Homo sapiens
aaaaaaactgaaggcacctatgact

SEQ ID NO: 338 - DNA, 25 bp, Homo sapiens
aaaaaactgaaggcacctatgactt

SEQ ID NO: 339 - DNA, 25 bp, Homo sapiens
aaaaactgaaggcacctatgactta

SEQ ID NO: 340 - DNA, 25 bp, Homo sapiens
aaaactgaaggcacctatgacttac

SEQ ID NO: 341 - DNA, 25 bp, Homo sapiens
aaactgaaggcacctatgacttacc

SEQ ID NO: 342 - DNA, 25 bp, Homo sapiens
aactgaaggcacctatgacttacct

SEQ ID NO: 343 - DNA, 25 bp, Homo sapiens
actgaaggcacctatgacttacctt

SEQ ID NO: 344 - DNA, 25 bp, Homo sapiens
ctgaaggcacctatgacttacctta

SEQ ID NO: 345 - DNA, 25 bp, Homo sapiens
tgaaggcacctatgacttaccttac

SEQ ID NO: 346 - DNA, 25 bp, Homo sapiens
gaaggcacctatgacttaccttact

SEQ ID NO: 347 - DNA, 25 bp, Homo sapiens
aaggcacctatgacttaccttactg

SEQ ID NO: 348 - DNA, 25 bp, Homo sapiens
aggcacctatgacttaccttactgg

SEQ ID NO: 349 - DNA, 25 bp, Homo sapiens
ggcacctatgacttaccttactggg

SEQ ID NO: 350 - DNA, 25 bp, Homo sapiens
gcacctatgacttaccttactggga

SEQ ID NO: 351 - DNA, 25 bp, Homo sapiens
cacctatgacttaccttactgggac

SEQ ID NO: 352 - DNA, 25 bp, Homo sapiens
acctatgacttaccttactgggacc

SEQ ID NO: 353 - DNA, 25 bp, Homo sapiens
cctatgacttaccttactgggaccg

SEQ ID NO: 354 - DNA, 25 bp, Homo sapiens
ctatgacttaccttactgggaccgg

SEQ ID NO: 355 - DNA, 25 bp, Homo sapiens
tatgacttaccttactgggaccggg

SEQ ID NO: 356 - DNA, 25 bp, Homo sapiens
atgacttaccttactgggaccgggc

SEQ ID NO: 357 - DNA, 25 bp, Homo sapiens
tgacttaccttactgggaccgggca

SEQ ID NO: 358 - DNA, 25 bp, Homo sapiens
gacttaccttactgggaccgggcag

SEQ ID NO: 359 - DNA, 25 bp, Homo sapiens
acttaccttactgggaccgggcagg

SEQ ID NO: 360 - DNA, 25 bp, Homo sapiens
cttaccttactgggaccgggcaggt

SEQ ID NO: 361 - DNA, 25 bp, Homo sapiens
ttaccttactgggaccgggcaggtt

SEQ ID NO: 362 - DNA, 25 bp, Homo sapiens
taccttactgggaccgggcaggttg

SEQ ID NO: 363 - DNA, 25 bp, Homo sapiens
accttactgggaccgggcaggttgg

SEQ ID NO: 364 - DNA, 25 bp, Homo sapiens
ccttactgggaccgggcaggttggt

SEQ ID NO: 365 - DNA, 25 bp, Homo sapiens
cttactgggaccgggcaggttggtg

SEQ ID NO: 366 - DNA, 25 bp, Homo sapiens
ttactgggaccgggcaggttggtgg

SEQ ID NO: 367 - DNA, 25 bp, Homo sapiens
tactgggaccgggcaggttggtgga

SEQ ID NO: 368 - DNA, 25 bp, Homo sapiens
actgggaccgggcaggttggtggaa

SEQ ID NO: 369 - DNA, 25 bp, Homo sapiens
ctgggaccgggcaggttggtggaaa

SEQ ID NO: 370 - DNA, 25 bp, Homo sapiens
tgggaccgggcaggttggtggaaag

SEQ ID NO: 371 - DNA, 25 bp, Homo sapiens
gggaccgggcaggttggtggaaagg

SEQ ID NO: 372 - DNA, 25 bp, Homo sapiens
ggaccgggcaggttggtggaaagga

SEQ ID NO: 373 - DNA, 25 bp, Homo sapiens
gaccgggcaggttggtggaaaggaa

SEQ ID NO: 374 - DNA, 25 bp, Homo sapiens
accgggcaggttggtggaaaggaat

SEQ ID NO: 375 - DNA, 25 bp, Homo sapiens
ccgggcaggttggtggaaaggaatg

SEQ ID NO: 376 - DNA, 25 bp, Homo sapiens
cgggcaggttggtggaaaggaatga

SEQ ID NO: 377 - DNA, 25 bp, Homo sapiens
gggcaggttggtggaaaggaatgaa

SEQ ID NO: 378 - DNA, 25 bp, Homo sapiens
ggcaggttggtggaaaggaatgaag

SEQ ID NO: 379 - DNA, 25 bp, Homo sapiens
gcaggttggtggaaaggaatgaagc

SEQ ID NO: 380 - DNA, 25 bp, Homo sapiens
caggttggtggaaaggaatgaagca

SEQ ID NO: 381 - DNA, 25 bp, Homo sapiens
aggttggtggaaaggaatgaagcag

SEQ ID NO: 382 - DNA, 25 bp, Homo sapiens
ggttggtggaaaggaatgaagcagt

SEQ ID NO: 383 - DNA, 25 bp, Homo sapiens
gttggtggaaaggaatgaagcagtt

SEQ ID NO: 384 - DNA, 25 bp, Homo sapiens
ttggtggaaaggaatgaagcagttt

SEQ ID NO: 385 - DNA, 25 bp, Homo sapiens
tggtggaaaggaatgaagcagtttc

SEQ ID NO: 386 - DNA, 25 bp, Homo sapiens
ggtggaaaggaatgaagcagtttct

SEQ ID NO: 387 - DNA, 25 bp, Homo sapiens
gtggaaaggaatgaagcagtttctt

SEQ ID NO: 388 - DNA, 25 bp, Homo sapiens
tggaaaggaatgaagcagtttcttc

SEQ ID NO: 389 - DNA, 25 bp, Homo sapiens
ggaaaggaatgaagcagtttcttcc

SEQ ID NO: 390 - DNA, 25 bp, Homo sapiens
gaaaggaatgaagcagtttcttcct

SEQ ID NO: 391 - DNA, 25 bp, Homo sapiens
aaaggaatgaagcagtttcttcctg

SEQ ID NO: 392 - DNA, 25 bp, Homo sapiens
aaggaatgaagcagtttcttcctgc

SEQ ID NO: 393 - DNA, 25 bp, Homo sapiens
aggaatgaagcagtttcttcctgca

SEQ ID NO: 394 - DNA, 25 bp, Homo sapiens
ggaatgaagcagtttcttcctgcaa

SEQ ID NO: 395 - DNA, 25 bp, Homo sapiens
gaatgaagcagtttcttcctgcaaa

SEQ ID NO: 396 - DNA, 25 bp, Homo sapiens
aatgaagcagtttcttcctgcaaaa

SEQ ID NO: 397 - DNA, 25 bp, Homo sapiens
atgaagcagtttcttcctgcaaaag

SEQ ID NO: 398 - DNA, 25 bp, Homo sapiens
tgaagcagtttcttcctgcaaaagc

SEQ ID NO: 399 - DNA, 25 bp, Homo sapiens
gaagcagtttcttcctgcaaaagca

SEQ ID NO: 400 - DNA, 25 bp, Homo sapiens
aagcagtttcttcctgcaaaagcag

SEQ ID NO: 401 - DNA, 25 bp, Homo sapiens
agcagtttcttcctgcaaaagcagt

SEQ ID NO: 402 - DNA, 25 bp, Homo sapiens
gcagtttcttcctgcaaaagcagtg

SEQ ID NO: 403 - DNA, 25 bp, Homo sapiens
cagtttcttcctgcaaaagcagtgg

SEQ ID NO: 404 - DNA, 25 bp, Homo sapiens
agtttcttcctgcaaaagcagtgga

SEQ ID NO: 405 - DNA, 25 bp, Homo sapiens
gtttcttcctgcaaaagcagtggac

SEQ ID NO: 406 - DNA, 25 bp, Homo sapiens
tttcttcctgcaaaagcagtggacc

SEQ ID NO: 407 - DNA, 25 bp, Homo sapiens
ttcttcctgcaaaagcagtggacca

SEQ ID NO: 408 - DNA, 25 bp, Homo sapiens
tcttcctgcaaaagcagtggaccat

SEQ ID NO: 409 - DNA, 25 bp, Homo sapiens
cttcctgcaaaagcagtggaccatg

SEQ ID NO: 410 - DNA, 25 bp, Homo sapiens
ttcctgcaaaagcagtggaccatga

SEQ ID NO: 411 - DNA, 25 bp, Homo sapiens
tcctgcaaaagcagtggaccatgag

SEQ ID NO: 412 - DNA, 25 bp, Homo sapiens
cctgcaaaagcagtggaccatgagg

SEQ ID NO: 413 - DNA, 25 bp, Homo sapiens
ctgcaaaagcagtggaccatgagga

SEQ ID NO: 414 - DNA, 25 bp, Homo sapiens
tgcaaaagcagtggaccatgaggaa

SEQ ID NO: 415 - DNA, 25 bp, Homo sapiens
gcaaaagcagtggaccatgaggaaa

SEQ ID NO: 416 - DNA, 25 bp, Homo sapiens
caaaagcagtggaccatgaggaaac

SEQ ID NO: 417 - DNA, 25 bp, Homo sapiens
aaaagcagtggaccatgaggaaacc

SEQ ID NO: 418 - DNA, 25 bp, Homo sapiens
aaagcagtggaccatgaggaaaccc

SEQ ID NO: 419 - DNA, 25 bp, Homo sapiens
aagcagtggaccatgaggaaacccc

SEQ ID NO: 420 - DNA, 25 bp, Homo sapiens
agcagtggaccatgaggaaacccca

SEQ ID NO: 421 - DNA, 25 bp, Homo sapiens
gcagtggaccatgaggaaaccccag

SEQ ID NO: 422 - DNA, 25 bp, Homo sapiens
cagtggaccatgaggaaaccccagt

SEQ ID NO: 423 - DNA, 25 bp, Homo sapiens
agtggaccatgaggaaaccccagtt

SEQ ID NO: 424 - DNA, 25 bp, Homo sapiens
gtggaccatgaggaaaccccagttc

SEQ ID NO: 425 - DNA, 25 bp, Homo sapiens
tggaccatgaggaaaccccagttcg

SEQ ID NO: 426 - DNA, 25 bp, Homo sapiens
ggaccatgaggaaaccccagttcgc

SEQ ID NO: 427 - DNA, 25 bp, Homo sapiens
gaccatgaggaaaccccagttcgct

SEQ ID NO: 428 - DNA, 25 bp, Homo sapiens
accatgaggaaaccccagttcgcta

SEQ ID NO: 429 - DNA, 25 bp, Homo sapiens
ccatgaggaaaccccagttcgctat

SEQ ID NO: 430 - DNA, 25 bp, Homo sapiens
catgaggaaaccccagttcgctata

SEQ ID NO: 431 - DNA, 25 bp, Homo sapiens
atgaggaaaccccagttcgctatag

SEQ ID NO: 432 - DNA, 25 bp, Homo sapiens
tgaggaaaccccagttcgctatagc

SEQ ID NO: 433 - DNA, 25 bp, Homo sapiens
gaggaaaccccagttcgctatagca

SEQ ID NO: 434 - DNA, 25 bp, Homo sapiens
aggaaaccccagttcgctatagcag

SEQ ID NO: 435 - DNA, 25 bp, Homo sapiens
ggaaaccccagttcgctatagcagc

SEQ ID NO: 436 - DNA, 25 bp, Homo sapiens
gaaaccccagttcgctatagcagca

SEQ ID NO: 437 - DNA, 25 bp, Homo sapiens
aaaccccagttcgctatagcagcag

SEQ ID NO: 438 - DNA, 25 bp, Homo sapiens
aaccccagttcgctatagcagcagc

SEQ ID NO: 439 - DNA, 25 bp, Homo sapiens
accccagttcgctatagcagcagcg

SEQ ID NO: 440 - DNA, 25 bp, Homo sapiens
ccccagttcgctatagcagcagcga

SEQ ID NO: 441 - DNA, 25 bp, Homo sapiens
cccagttcgctatagcagcagcgaa

SEQ ID NO: 442 - DNA, 25 bp, Homo sapiens
ccagttcgctatagcagcagcgaag

SEQ ID NO: 443 - DNA, 25 bp, Homo sapiens
cagttcgctatagcagcagcgaagt

SEQ ID NO: 444 - DNA, 25 bp, Homo sapiens
agttcgctatagcagcagcgaagtt

SEQ ID NO: 445 - DNA, 25 bp, Homo sapiens
gttcgctatagcagcagcgaagtta

SEQ ID NO: 446 - DNA, 25 bp, Homo sapiens
ttcgctatagcagcagcgaagttaa

SEQ ID NO: 447 - DNA, 25 bp, Homo sapiens
tcgctatagcagcagcgaagttaat

SEQ ID NO: 448 - DNA, 25 bp, Homo sapiens
cgctatagcagcagcgaagttaatc

SEQ ID NO: 449 - DNA, 25 bp, Homo sapiens
gctatagcagcagcgaagttaatca

SEQ ID NO: 450 - DNA, 25 bp, Homo sapiens
ctatagcagcagcgaagttaatcac

SEQ ID NO: 451 - DNA, 25 bp, Homo sapiens
tatagcagcagcgaagttaatcacc

SEQ ID NO: 452 - DNA, 25 bp, Homo sapiens
atagcagcagcgaagttaatcacct

SEQ ID NO: 453 - DNA, 25 bp, Homo sapiens
tagcagcagcgaagttaatcacctg

SEQ ID NO: 454 - DNA, 25 bp, Homo sapiens
agcagcagcgaagttaatcacctga

SEQ ID NO: 455 - DNA, 25 bp, Homo sapiens
gcagcagcgaagttaatcacctgag

SEQ ID NO: 456 - DNA, 25 bp, Homo sapiens
cagcagcgaagttaatcacctgagt

SEQ ID NO: 457 - DNA, 25 bp, Homo sapiens
agcagcgaagttaatcacctgagtc

SEQ ID NO: 458 - DNA, 25 bp, Homo sapiens
gcagcgaagttaatcacctgagtcc

SEQ ID NO: 459 - DNA, 25 bp, Homo sapiens
cagcgaagttaatcacctgagtcca

SEQ ID NO: 460 - DNA, 25 bp, Homo sapiens
agcgaagttaatcacctgagtccaa

SEQ ID NO: 461 - DNA, 25 bp, Homo sapiens
gcgaagttaatcacctgagtccaag

SEQ ID NO: 462 - DNA, 25 bp, Homo sapiens
cgaagttaatcacctgagtccaaga

SEQ ID NO: 463 - DNA, 25 bp, Homo sapiens
gaagttaatcacctgagtccaagag

SEQ ID NO: 464 - DNA, 25 bp, Homo sapiens
aagttaatcacctgagtccaagaga

SEQ ID NO: 465 - DNA, 25 bp, Homo sapiens
agttaatcacctgagtccaagagaa

SEQ ID NO: 466 - DNA, 25 bp, Homo sapiens
gttaatcacctgagtccaagagaag

SEQ ID NO: 467 - DNA, 25 bp, Homo sapiens
ttaatcacctgagtccaagagaagt

SEQ ID NO: 468 - DNA, 25 bp, Homo sapiens
taatcacctgagtccaagagaagtc

SEQ ID NO: 469 - DNA, 25 bp, Homo sapiens
aatcacctgagtccaagagaagtca

SEQ ID NO: 470 - DNA, 25 bp, Homo sapiens
atcacctgagtccaagagaagtcac

SEQ ID NO: 471 - DNA, 25 bp, Homo sapiens
tcacctgagtccaagagaagtcacc

SEQ ID NO: 472 - DNA, 25 bp, Homo sapiens
cacctgagtccaagagaagtcacca

SEQ ID NO: 473 - DNA, 25 bp, Homo sapiens
acctgagtccaagagaagtcaccac

SEQ ID NO: 474 - DNA, 25 bp, Homo sapiens
cctgagtccaagagaagtcaccaca

SEQ ID NO: 475 - DNA, 25 bp, Homo sapiens
ctgagtccaagagaagtcaccacag

SEQ ID NO: 476 - DNA, 25 bp, Homo sapiens
tgagtccaagagaagtcaccacagt

SEQ ID NO: 477 - DNA, 25 bp, Homo sapiens
gagtccaagagaagtcaccacagtg

SEQ ID NO: 478 - DNA, 25 bp, Homo sapiens
agtccaagagaagtcaccacagtgc

SEQ ID NO: 479 - DNA, 25 bp, Homo sapiens
gtccaagagaagtcaccacagtgct

SEQ ID NO: 480 - DNA, 25 bp, Homo sapiens
tccaagagaagtcaccacagtgctg

SEQ ID NO: 481 - DNA, 25 bp, Homo sapiens
ccaagagaagtcaccacagtgctgc

SEQ ID NO: 482 - DNA, 25 bp, Homo sapiens
caagagaagtcaccacagtgctgca

SEQ ID NO: 483 - DNA, 25 bp, Homo sapiens
aagagaagtcaccacagtgctgcag

SEQ ID NO: 484 - DNA, 25 bp, Homo sapiens
agagaagtcaccacagtgctgcagg

SEQ ID NO: 485 - DNA, 25 bp, Homo sapiens
gagaagtcaccacagtgctgcaggc

SEQ ID NO: 486 - DNA, 25 bp, Homo sapiens
agaagtcaccacagtgctgcaggct

SEQ ID NO: 487 - DNA, 25 bp, Homo sapiens
gaagtcaccacagtgctgcaggctg

SEQ ID NO: 488 - DNA, 25 bp, Homo sapiens
aagtcaccacagtgctgcaggctga

SEQ ID NO: 489 - DNA, 25 bp, Homo sapiens
agtcaccacagtgctgcaggctgac

SEQ ID NO: 490 - DNA, 25 bp, Homo sapiens
gtcaccacagtgctgcaggctgact

SEQ ID NO: 491 - DNA, 25 bp, Homo sapiens
tcaccacagtgctgcaggctgactc

SEQ ID NO: 492 - DNA, 25 bp, Homo sapiens
caccacagtgctgcaggctgactct

SEQ ID NO: 493 - DNA, 25 bp, Homo sapiens
accacagtgctgcaggctgactctg

SEQ ID NO: 494 - DNA, 25 bp, Homo sapiens
ccacagtgctgcaggctgactctgc

SEQ ID NO: 495 - DNA, 25 bp, Homo sapiens
cacagtgctgcaggctgactctgca

SEQ ID NO: 496 - DNA, 25 bp, Homo sapiens
acagtgctgcaggctgactctgcag

SEQ ID NO: 497 - DNA, 25 bp, Homo sapiens
cagtgctgcaggctgactctgcaga

SEQ ID NO: 498 - DNA, 25 bp, Homo sapiens
agtgctgcaggctgactctgcagag

SEQ ID NO: 499 - DNA, 25 bp, Homo sapiens
gtgctgcaggctgactctgcagagt

SEQ ID NO: 500 - DNA, 25 bp, Homo sapiens
tgctgcaggctgactctgcagagta

SEQ ID NO: 501 - DNA, 25 bp, Homo sapiens
gctgcaggctgactctgcagagtat

SEQ ID NO: 502 - DNA, 25 bp, Homo sapiens
ctgcaggctgactctgcagagtatg

SEQ ID NO: 503 - DNA, 25 bp, Homo sapiens
tgcaggctgactctgcagagtatgc

SEQ ID NO: 504 - DNA, 25 bp, Homo sapiens
gcaggctgactctgcagagtatgct

SEQ ID NO: 505 - DNA, 25 bp, Homo sapiens
caggctgactctgcagagtatgctc

SEQ ID NO: 506 - DNA, 25 bp, Homo sapiens
aggctgactctgcagagtatgctca

SEQ ID NO: 507 - DNA, 25 bp, Homo sapiens
ggctgactctgcagagtatgctcag

SEQ ID NO: 508 - DNA, 25 bp, Homo sapiens
gctgactctgcagagtatgctcagc

SEQ ID NO: 509 - DNA, 25 bp, Homo sapiens
ctgactctgcagagtatgctcagcc

SEQ ID NO: 510 - DNA, 25 bp, Homo sapiens
tgactctgcagagtatgctcagcca

SEQ ID NO: 511 - DNA, 25 bp, Homo sapiens
gactctgcagagtatgctcagccac

SEQ ID NO: 512 - DNA, 25 bp, Homo sapiens
actctgcagagtatgctcagccact

SEQ ID NO: 513 - DNA, 25 bp, Homo sapiens
ctctgcagagtatgctcagccactg

SEQ ID NO: 514 - DNA, 25 bp, Homo sapiens
tctgcagagtatgctcagccactgg

SEQ ID NO: 515 - DNA, 25 bp, Homo sapiens
ctgcagagtatgctcagccactggt

SEQ ID NO: 516 - DNA, 25 bp, Homo sapiens
tgcagagtatgctcagccactggta

SEQ ID NO: 517 - DNA, 25 bp, Homo sapiens
gcagagtatgctcagccactggtag

SEQ ID NO: 518 - DNA, 25 bp, Homo sapiens
cagagtatgctcagccactggtagg

SEQ ID NO: 519 - DNA, 25 bp, Homo sapiens
agagtatgctcagccactggtagga

SEQ ID NO: 520 - DNA, 25 bp, Homo sapiens
gagtatgctcagccactggtaggag

SEQ ID NO: 521 - DNA, 25 bp, Homo sapiens
agtatgctcagccactggtaggagg

SEQ ID NO: 522 - DNA, 25 bp, Homo sapiens
gtatgctcagccactggtaggagga

SEQ ID NO: 523 - DNA, 25 bp, Homo sapiens
tatgctcagccactggtaggaggaa

SEQ ID NO: 524 - DNA, 25 bp, Homo sapiens
atgctcagccactggtaggaggaat

SEQ ID NO: 525 - DNA, 25 bp, Homo sapiens
tgctcagccactggtaggaggaatt

SEQ ID NO: 526 - DNA, 25 bp, Homo sapiens
gctcagccactggtaggaggaattg

SEQ ID NO: 527 - DNA, 25 bp, Homo sapiens
ctcagccactggtaggaggaattgt

SEQ ID NO: 528 - DNA, 25 bp, Homo sapiens
tcagccactggtaggaggaattgtt

SEQ ID NO: 529 - DNA, 25 bp, Homo sapiens
cagccactggtaggaggaattgttg

SEQ ID NO: 530 - DNA, 25 bp, Homo sapiens
agccactggtaggaggaattgttgg

SEQ ID NO: 531 - DNA, 25 bp, Homo sapiens
gccactggtaggaggaattgttggt

SEQ ID NO: 532 - DNA, 25 bp, Homo sapiens
ccactggtaggaggaattgttggta

SEQ ID NO: 533 - DNA, 25 bp, Homo sapiens
cactggtaggaggaattgttggtac

SEQ ID NO: 534 - DNA, 25 bp, Homo sapiens
actggtaggaggaattgttggtaca

SEQ ID NO: 535 - DNA, 25 bp, Homo sapiens
ctggtaggaggaattgttggtacac

SEQ ID NO: 536 - DNA, 25 bp, Homo sapiens
tggtaggaggaattgttggtacact

SEQ ID NO: 537 - DNA, 25 bp, Homo sapiens
ggtaggaggaattgttggtacactt

SEQ ID NO: 538 - DNA, 25 bp, Homo sapiens
gtaggaggaattgttggtacacttc

SEQ ID NO: 539 - DNA, 25 bp, Homo sapiens
taggaggaattgttggtacacttca

SEQ ID NO: 540 - DNA, 25 bp, Homo sapiens
aggaggaattgttggtacacttcat

SEQ ID NO: 541 - DNA, 25 bp, Homo sapiens
ggaggaattgttggtacacttcatc

SEQ ID NO: 542 - DNA, 25 bp, Homo sapiens
gaggaattgttggtacacttcatca

SEQ ID NO: 543 - DNA, 25 bp, Homo sapiens
aggaattgttggtacacttcatcaa

SEQ ID NO: 544 - DNA, 25 bp, Homo sapiens
ggaattgttggtacacttcatcaaa

SEQ ID NO: 545 - DNA, 25 bp, Homo sapiens
gaattgttggtacacttcatcaaag

SEQ ID NO: 546 - DNA, 25 bp, Homo sapiens
aattgttggtacacttcatcaaaga

SEQ ID NO: 547 - DNA, 25 bp, Homo sapiens
attgttggtacacttcatcaaagat

SEQ ID NO: 548 - DNA, 25 bp, Homo sapiens
ttgttggtacacttcatcaaagatc

SEQ ID NO: 549 - DNA, 25 bp, Homo sapiens
tgttggtacacttcatcaaagatct

SEQ ID NO: 550 - DNA, 25 bp, Homo sapiens
gttggtacacttcatcaaagatcta

SEQ ID NO: 551 - DNA, 25 bp, Homo sapiens
ttggtacacttcatcaaagatctac

SEQ ID NO: 552 - DNA, 25 bp, Homo sapiens
tggtacacttcatcaaagatctacc

SEQ ID NO: 553 - DNA, 25 bp, Homo sapiens
ggtacacttcatcaaagatctacct

SEQ ID NO: 554 - DNA, 25 bp, Homo sapiens
gtacacttcatcaaagatctacctt

SEQ ID NO: 555 - DNA, 25 bp, Homo sapiens
tacacttcatcaaagatctaccttt

SEQ ID NO: 556 - DNA, 25 bp, Homo sapiens
acacttcatcaaagatctaccttta

SEQ ID NO: 557 - DNA, 25 bp, Homo sapiens
cacttcatcaaagatctacctttaa

SEQ ID NO: 558 - DNA, 25 bp, Homo sapiens
acttcatcaaagatctacctttaaa

SEQ ID NO: 559 - DNA, 25 bp, Homo sapiens
cttcatcaaagatctacctttaaac

SEQ ID NO: 560 - DNA, 25 bp, Homo sapiens
ttcatcaaagatctacctttaaacc

SEQ ID NO: 561 - DNA, 25 bp, Homo sapiens
tcatcaaagatctacctttaaacca

SEQ ID NO: 562 - DNA, 25 bp, Homo sapiens
catcaaagatctacctttaaaccag

SEQ ID NO: 563 - DNA, 25 bp, Homo sapiens
atcaaagatctacctttaaaccaga

SEQ ID NO: 564 - DNA, 25 bp, Homo sapiens
tcaaagatctacctttaaaccagaa

SEQ ID NO: 565 - DNA, 25 bp, Homo sapiens
caaagatctacctttaaaccagaag

SEQ ID NO: 566 - DNA, 25 bp, Homo sapiens
aaagatctacctttaaaccagaaga

SEQ ID NO: 567 - DNA, 25 bp, Homo sapiens
aagatctacctttaaaccagaagaa

SEQ ID NO: 568 - DNA, 25 bp, Homo sapiens
agatctacctttaaaccagaagaag

SEQ ID NO: 569 - DNA, 25 bp, Homo sapiens
gatctacctttaaaccagaagaagg

SEQ ID NO: 570 - DNA, 25 bp, Homo sapiens
atctacctttaaaccagaagaagga

SEQ ID NO: 571 - DNA, 25 bp, Homo sapiens
tctacctttaaaccagaagaaggaa

SEQ ID NO: 572 - DNA, 25 bp, Homo sapiens
ctacctttaaaccagaagaaggaaa

SEQ ID NO: 573 - DNA, 25 bp, Homo sapiens
tacctttaaaccagaagaaggaaaa

SEQ ID NO: 574 - DNA, 25 bp, Homo sapiens
acctttaaaccagaagaaggaaaag

SEQ ID NO: 575 - DNA, 25 bp, Homo sapiens
cctttaaaccagaagaaggaaaaga

SEQ ID NO: 576 - DNA, 25 bp, Homo sapiens
ctttaaaccagaagaaggaaaagaa

SEQ ID NO: 577 - DNA, 25 bp, Homo sapiens
tttaaaccagaagaaggaaaagaag

SEQ ID NO: 578 - DNA, 25 bp, Homo sapiens
ttaaaccagaagaaggaaaagaagc

SEQ ID NO: 579 - DNA, 25 bp, Homo sapiens
taaaccagaagaaggaaaagaagca

SEQ ID NO: 580 - DNA, 25 bp, Homo sapiens
aaaccagaagaaggaaaagaagcag

SEQ ID NO: 581 - DNA, 25 bp, Homo sapiens
aaccagaagaaggaaaagaagcagg

SEQ ID NO: 582 - DNA, 25 bp, Homo sapiens
accagaagaaggaaaagaagcaggc

SEQ ID NO: 583 - DNA, 25 bp, Homo sapiens
ccagaagaaggaaaagaagcaggct

SEQ ID NO: 584 - DNA, 25 bp, Homo sapiens
cagaagaaggaaaagaagcaggcta

SEQ ID NO: 585 - DNA, 25 bp, Homo sapiens
agaagaaggaaaagaagcaggctat

SEQ ID NO: 586 - DNA, 25 bp, Homo sapiens
gaagaaggaaaagaagcaggctatg

SEQ ID NO: 587 - DNA, 25 bp, Homo sapiens
aagaaggaaaagaagcaggctatgc

SEQ ID NO: 588 - DNA, 25 bp, Homo sapiens
agaaggaaaagaagcaggctatgca

SEQ ID NO: 589 - DNA, 25 bp, Homo sapiens
gaaggaaaagaagcaggctatgcag

SEQ ID NO: 590 - DNA, 25 bp, Homo sapiens
aaggaaaagaagcaggctatgcaga

SEQ ID NO: 591 - DNA, 25 bp, Homo sapiens
aggaaaagaagcaggctatgcagac

SEQ ID NO: 592 - DNA, 25 bp, Homo sapiens
ggaaaagaagcaggctatgcagacc

SEQ ID NO: 593 - DNA, 25 bp, Homo sapiens
gaaaagaagcaggctatgcagacct

SEQ ID NO: 594 - DNA, 25 bp, Homo sapiens
aaaagaagcaggctatgcagaccta

SEQ ID NO: 595 - DNA, 25 bp, Homo sapiens
aaagaagcaggctatgcagacctag

SEQ ID NO: 596 - DNA, 25 bp, Homo sapiens
aagaagcaggctatgcagacctaga

SEQ ID NO: 597 - DNA, 25 bp, Homo sapiens
agaagcaggctatgcagacctagat

SEQ ID NO: 598 - DNA, 25 bp, Homo sapiens
gaagcaggctatgcagacctagatc

SEQ ID NO: 599 - DNA, 25 bp, Homo sapiens
aagcaggctatgcagacctagatcc

SEQ ID NO: 600 - DNA, 25 bp, Homo sapiens
agcaggctatgcagacctagatcct

SEQ ID NO: 601 - DNA, 25 bp, Homo sapiens
gcaggctatgcagacctagatcctt

SEQ ID NO: 602 - DNA, 25 bp, Homo sapiens
caggctatgcagacctagatcctta

SEQ ID NO: 603 - DNA, 17 bp, Homo sapiens
caacttcagttggtcag

SEQ ID NO: 604 - DNA, 17 bp, Homo sapiens
aacttcagttggtcagc

SEQ ID NO: 605 - DNA, 17 bp, Homo sapiens
acttcagttggtcagcc

SEQ ID NO: 606 - DNA, 17 bp, Homo sapiens
cttcagttggtcagccc

SEQ ID NO: 607 - DNA, 17 bp, Homo sapiens
ttcagttggtcagccct

SEQ ID NO: 608 - DNA, 17 bp, Homo sapiens
tcagttggtcagccctc

SEQ ID NO: 609 - DNA, 17 bp, Homo sapiens
cagttggtcagccctcc

SEQ ID NO: 610 - DNA, 17 bp, Homo sapiens
agttggtcagccctcca

SEQ ID NO: 611 - DNA, 17 bp, Homo sapiens
gttggtcagccctccac

SEQ ID NO: 612 - DNA, 17 bp, Homo sapiens
ttggtcagccctccaca

SEQ ID NO: 613 - DNA, 17 bp, Homo sapiens
tggtcagccctccacat

SEQ ID NO: 614 - DNA, 17 bp, Homo sapiens
ggtcagccctccacatc

SEQ ID NO: 615 - DNA, 17 bp, Homo sapiens
gtcagccctccacatcc

SEQ ID NO: 616 - DNA, 17 bp, Homo sapiens
tcagccctccacatcca

SEQ ID NO: 617 - DNA, 17 bp, Homo sapiens
cagccctccacatccac

SEQ ID NO: 618 - DNA, 17 bp, Homo sapiens
agccctccacatccact

SEQ ID NO: 619 - DNA, 17 bp, Homo sapiens
gccctccacatccactt

SEQ ID NO: 620 - DNA, 17 bp, Homo sapiens
ccctccacatccacttt

SEQ ID NO: 621 - DNA, 17 bp, Homo sapiens
cctccacatccactttc

SEQ ID NO: 622 - DNA, 17 bp, Homo sapiens
ctccacatccactttca

SEQ ID NO: 623 - DNA, 17 bp, Homo sapiens
tccacatccactttcaa

SEQ ID NO: 624 - DNA, 17 bp, Homo sapiens
ccacatccactttcaag

SEQ ID NO: 625 - DNA, 17 bp, Homo sapiens
cacatccactttcaagg

SEQ ID NO: 626 - DNA, 17 bp, Homo sapiens
acatccactttcaaggc

SEQ ID NO: 627 - DNA, 17 bp, Homo sapiens
catccactttcaaggct

SEQ ID NO: 628 - DNA, 17 bp, Homo sapiens
atccactttcaaggcta

SEQ ID NO: 629 - DNA, 17 bp, Homo sapiens
tccactttcaaggctac

SEQ ID NO: 630 - DNA, 17 bp, Homo sapiens
ccactttcaaggctacg

SEQ ID NO: 631 - DNA, 17 bp, Homo sapiens
cactttcaaggctacgg

SEQ ID NO: 632 - DNA, 17 bp, Homo sapiens
actttcaaggctacggg

SEQ ID NO: 633 - DNA, 17 bp, Homo sapiens
ctttcaaggctacgggg

SEQ ID NO: 634 - DNA, 17 bp, Homo sapiens
tttcaaggctacgggga

SEQ ID NO: 635 - DNA, 17 bp, Homo sapiens
ttcaaggctacggggaa

SEQ ID NO: 636 - DNA, 17 bp, Homo sapiens
tcaaggctacggggaac

SEQ ID NO: 637 - DNA, 17 bp, Homo sapiens
caaggctacggggaacc

SEQ ID NO: 638 - DNA, 17 bp, Homo sapiens
aaggctacggggaacca

SEQ ID NO: 639 - DNA, 17 bp, Homo sapiens
aggctacggggaaccaa

SEQ ID NO: 640 - DNA, 17 bp, Homo sapiens
ggctacggggaaccaac

SEQ ID NO: 641 - DNA, 17 bp, Homo sapiens
gctacggggaaccaacc

SEQ ID NO: 642 - DNA, 17 bp, Homo sapiens
ctacggggaaccaacct

SEQ ID NO: 643 - DNA, 17 bp, Homo sapiens
tacggggaaccaacctc

SEQ ID NO: 644 - DNA, 17 bp, Homo sapiens
acggggaaccaacctcc

SEQ ID NO: 645 - DNA, 17 bp, Homo sapiens
cggggaaccaacctccc

SEQ ID NO: 646 - DNA, 17 bp, Homo sapiens
ggggaaccaacctcccc

SEQ ID NO: 647 - DNA, 17 bp, Homo sapiens
gggaaccaacctccccc

SEQ ID NO: 648 - DNA, 17 bp, Homo sapiens
ggaaccaacctccccca

SEQ ID NO: 649 - DNA, 17 bp, Homo sapiens
gaaccaacctcccccac

SEQ ID NO: 650 - DNA, 17 bp, Homo sapiens
aaccaacctcccccact

SEQ ID NO: 651 - DNA, 17 bp, Homo sapiens
accaacctcccccacta

SEQ ID NO: 652 - DNA, 17 bp, Homo sapiens
ccaacctcccccactag

SEQ ID NO: 653 - DNA, 17 bp, Homo sapiens
caacctcccccactagt

SEQ ID NO: 654 - DNA, 17 bp, Homo sapiens
aacctcccccactagtg

SEQ ID NO: 655 - DNA, 17 bp, Homo sapiens
acctcccccactagtgg

SEQ ID NO: 656 - DNA, 17 bp, Homo sapiens
cctcccccactagtggg

SEQ ID NO: 657 - DNA, 17 bp, Homo sapiens
ctcccccactagtggga

SEQ ID NO: 658 - DNA, 17 bp, Homo sapiens
tcccccactagtgggaa

SEQ ID NO: 659 - DNA, 17 bp, Homo sapiens
cccccactagtgggaac

SEQ ID NO: 660 - DNA, 17 bp, Homo sapiens
ccccactagtgggaact

SEQ ID NO: 661 - DNA, 17 bp, Homo sapiens
cccactagtgggaactt

SEQ ID NO: 662 - DNA, 17 bp, Homo sapiens
ccactagtgggaactta

SEQ ID NO: 663 - DNA, 17 bp, Homo sapiens
cactagtgggaacttac

SEQ ID NO: 664 - DNA, 17 bp, Homo sapiens
actagtgggaacttaca

SEQ ID NO: 665 - DNA, 17 bp, Homo sapiens
ctagtgggaacttacaa

SEQ ID NO: 666 - DNA, 17 bp, Homo sapiens
tagtgggaacttacaat

SEQ ID NO: 667 - DNA, 17 bp, Homo sapiens
agtgggaacttacaata

SEQ ID NO: 668 - DNA, 17 bp, Homo sapiens
gtgggaacttacaatac

SEQ ID NO: 669 - DNA, 17 bp, Homo sapiens
tgggaacttacaataca

SEQ ID NO: 670 - DNA, 17 bp, Homo sapiens
gggaacttacaatacac

SEQ ID NO: 671 - DNA, 17 bp, Homo sapiens
ggaacttacaatacact

SEQ ID NO: 672 - DNA, 17 bp, Homo sapiens
gaacttacaatacactt

SEQ ID NO: 673 - DNA, 17 bp, Homo sapiens
aacttacaatacacttc

SEQ ID NO: 674 - DNA, 17 bp, Homo sapiens
acttacaatacacttct

SEQ ID NO: 675 - DNA, 17 bp, Homo sapiens
cttacaatacacttctc

SEQ ID NO: 676 - DNA, 17 bp, Homo sapiens
ttacaatacacttctct

SEQ ID NO: 677 - DNA, 17 bp, Homo sapiens
tacaatacacttctctc

SEQ ID NO: 678 - DNA, 17 bp, Homo sapiens
acaatacacttctctcc

SEQ ID NO: 679 - DNA, 17 bp, Homo sapiens
caatacacttctctcca

SEQ ID NO: 680 - DNA, 17 bp, Homo sapiens
aatacacttctctccag

SEQ ID NO: 681 - DNA, 17 bp, Homo sapiens
atacacttctctccagg

SEQ ID NO: 682 - DNA, 17 bp, Homo sapiens
tacacttctctccagga

SEQ ID NO: 683 - DNA, 17 bp, Homo sapiens
acacttctctccaggac

SEQ ID NO: 684 - DNA, 17 bp, Homo sapiens
cacttctctccaggact

SEQ ID NO: 685 - DNA, 17 bp, Homo sapiens
acttctctccaggactg

SEQ ID NO: 686 - DNA, 17 bp, Homo sapiens
cttctctccaggactga

SEQ ID NO: 687 - DNA, 17 bp, Homo sapiens
ttctctccaggactgac

SEQ ID NO: 688 - DNA, 17 bp, Homo sapiens
tctctccaggactgaca

SEQ ID NO: 689 - DNA, 17 bp, Homo sapiens
ctctccaggactgacag

SEQ ID NO: 690 - DNA, 17 bp, Homo sapiens
tctccaggactgacagc

SEQ ID NO: 691 - DNA, 17 bp, Homo sapiens
ctccaggactgacagct

SEQ ID NO: 692 - DNA, 17 bp, Homo sapiens
tccaggactgacagctg

SEQ ID NO: 693 - DNA, 17 bp, Homo sapiens
ccaggactgacagctgc

SEQ ID NO: 694 - DNA, 17 bp, Homo sapiens
caggactgacagctgct

SEQ ID NO: 695 - DNA, 17 bp, Homo sapiens
aggactgacagctgctc

SEQ ID NO: 696 - DNA, 17 bp, Homo sapiens
ggactgacagctgctcc

SEQ ID NO: 697 - DNA, 17 bp, Homo sapiens
gactgacagctgctcct

SEQ ID NO: 698 - DNA, 17 bp, Homo sapiens
actgacagctgctcctc

SEQ ID NO: 699 - DNA, 17 bp, Homo sapiens
ctgacagctgctcctca

SEQ ID NO: 700 - DNA, 17 bp, Homo sapiens
tgacagctgctcctcag

SEQ ID NO: 701 - DNA, 17 bp, Homo sapiens
gacagctgctcctcagc

SEQ ID NO: 702 - DNA, 17 bp, Homo sapiens
acagctgctcctcagcc

SEQ ID NO: 703 - DNA, 17 bp, Homo sapiens
cagctgctcctcagccc

SEQ ID NO: 704 - DNA, 17 bp, Homo sapiens
agctgctcctcagccca

SEQ ID NO: 705 - DNA, 17 bp, Homo sapiens
gctgctcctcagcccag

SEQ ID NO: 706 - DNA, 17 bp, Homo sapiens
ctgctcctcagcccagg

SEQ ID NO: 707 - DNA, 17 bp, Homo sapiens
tgctcctcagcccaggc

SEQ ID NO: 708 - DNA, 17 bp, Homo sapiens
gctcctcagcccaggcc

SEQ ID NO: 709 - DNA, 17 bp, Homo sapiens
ctcctcagcccaggccc

SEQ ID NO: 710 - DNA, 17 bp, Homo sapiens
tcctcagcccaggccca

SEQ ID NO: 711 - DNA, 17 bp, Homo sapiens
cctcagcccaggcccag

SEQ ID NO: 712 - DNA, 17 bp, Homo sapiens
ctcagcccaggcccagt

SEQ ID NO: 713 - DNA, 17 bp, Homo sapiens
tcagcccaggcccagta

SEQ ID NO: 714 - DNA, 17 bp, Homo sapiens
cagcccaggcccagtat

SEQ ID NO: 715 - DNA, 17 bp, Homo sapiens
agcccaggcccagtatg

SEQ ID NO: 716 - DNA, 17 bp, Homo sapiens
gcccaggcccagtatga

SEQ ID NO: 717 - DNA, 17 bp, Homo sapiens
cccaggcccagtatgat

SEQ ID NO: 718 - DNA, 17 bp, Homo sapiens
ccaggcccagtatgata

SEQ ID NO: 719 - DNA, 17 bp, Homo sapiens
caggcccagtatgatac

SEQ ID NO: 720 - DNA, 17 bp, Homo sapiens
aggcccagtatgatacc

SEQ ID NO: 721 - DNA, 17 bp, Homo sapiens
ggcccagtatgataccc

SEQ ID NO: 722 - DNA, 17 bp, Homo sapiens
gcccagtatgatacccc

SEQ ID NO: 723 - DNA, 17 bp, Homo sapiens
cccagtatgataccccg

SEQ ID NO: 724 - DNA, 17 bp, Homo sapiens
ccagtatgataccccga

SEQ ID NO: 725 - DNA, 17 bp, Homo sapiens
cagtatgataccccgaa

SEQ ID NO: 726 - DNA, 17 bp, Homo sapiens
agtatgataccccgaaa

SEQ ID NO: 727 - DNA, 17 bp, Homo sapiens
gtatgataccccgaaag

SEQ ID NO: 728 - DNA, 17 bp, Homo sapiens
tatgataccccgaaagc

SEQ ID NO: 729 - DNA, 17 bp, Homo sapiens
atgataccccgaaagct

SEQ ID NO: 730 - DNA, 17 bp, Homo sapiens
tgataccccgaaagctg

SEQ ID NO: 731 - DNA, 17 bp, Homo sapiens
gataccccgaaagctgg

SEQ ID NO: 732 - DNA, 17 bp, Homo sapiens
ataccccgaaagctggg

SEQ ID NO: 733 - DNA, 17 bp, Homo sapiens
taccccgaaagctggga

SEQ ID NO: 734 - DNA, 17 bp, Homo sapiens
accccgaaagctgggaa

SEQ ID NO: 735 - DNA, 17 bp, Homo sapiens
ccccgaaagctgggaag

SEQ ID NO: 736 - DNA, 17 bp, Homo sapiens
cccgaaagctgggaagc

SEQ ID NO: 737 - DNA, 17 bp, Homo sapiens
ccgaaagctgggaagcc

SEQ ID NO: 738 - DNA, 17 bp, Homo sapiens
cgaaagctgggaagcca

SEQ ID NO: 739 - DNA, 17 bp, Homo sapiens
gaaagctgggaagccag

SEQ ID NO: 740 - DNA, 17 bp, Homo sapiens
aaagctgggaagccagg

SEQ ID NO: 741 - DNA, 17 bp, Homo sapiens
aagctgggaagccaggt

SEQ ID NO: 742 - DNA, 17 bp, Homo sapiens
agctgggaagccaggtc

SEQ ID NO: 743 - DNA, 17 bp, Homo sapiens
gctgggaagccaggtct

SEQ ID NO: 744 - DNA, 17 bp, Homo sapiens
ctgggaagccaggtcta

SEQ ID NO: 745 - DNA, 17 bp, Homo sapiens
tgggaagccaggtctac

SEQ ID NO: 746 - DNA, 17 bp, Homo sapiens
gggaagccaggtctacc

SEQ ID NO: 747 - DNA, 17 bp, Homo sapiens
ggaagccaggtctacct

SEQ ID NO: 748 - DNA, 17 bp, Homo sapiens
gaagccaggtctacctg

SEQ ID NO: 749 - DNA, 17 bp, Homo sapiens
aagccaggtctacctgc

SEQ ID NO: 750 - DNA, 17 bp, Homo sapiens
agccaggtctacctgcc

SEQ ID NO: 751 - DNA, 17 bp, Homo sapiens
gccaggtctacctgccc

SEQ ID NO: 752 - DNA, 17 bp, Homo sapiens
ccaggtctacctgcccc

SEQ ID NO: 753 - DNA, 17 bp, Homo sapiens
caggtctacctgcccca

SEQ ID NO: 754 - DNA, 17 bp, Homo sapiens
aggtctacctgccccag

SEQ ID NO: 755 - DNA, 17 bp, Homo sapiens
ggtctacctgccccaga

SEQ ID NO: 756 - DNA, 17 bp, Homo sapiens
gtctacctgccccagac

SEQ ID NO: 757 - DNA, 17 bp, Homo sapiens
tctacctgccccagacg

SEQ ID NO: 758 - DNA, 17 bp, Homo sapiens
ctacctgccccagacga

SEQ ID NO: 759 - DNA, 17 bp, Homo sapiens
tacctgccccagacgaa

SEQ ID NO: 760 - DNA, 17 bp, Homo sapiens
acctgccccagacgaat

SEQ ID NO: 761 - DNA, 17 bp, Homo sapiens
cctgccccagacgaatt

SEQ ID NO: 762 - DNA, 17 bp, Homo sapiens
ctgccccagacgaattg

SEQ ID NO: 763 - DNA, 17 bp, Homo sapiens
tgccccagacgaattgg

SEQ ID NO: 764 - DNA, 17 bp, Homo sapiens
gccccagacgaattggt

SEQ ID NO: 765 - DNA, 17 bp, Homo sapiens
ccccagacgaattggtg

SEQ ID NO: 766 - DNA, 17 bp, Homo sapiens
cccagacgaattggtgt

SEQ ID NO: 767 - DNA, 17 bp, Homo sapiens
ccagacgaattggtgta

SEQ ID NO: 768 - DNA, 17 bp, Homo sapiens
cagacgaattggtgtac

SEQ ID NO: 769 - DNA, 17 bp, Homo sapiens
agacgaattggtgtacc

SEQ ID NO: 770 - DNA, 17 bp, Homo sapiens
gacgaattggtgtacca

SEQ ID NO: 771 - DNA, 17 bp, Homo sapiens
acgaattggtgtaccag

SEQ ID NO: 772 - DNA, 17 bp, Homo sapiens
cgaattggtgtaccagg

SEQ ID NO: 773 - DNA, 17 bp, Homo sapiens
gaattggtgtaccaggt

SEQ ID NO: 774 - DNA, 17 bp, Homo sapiens
aattggtgtaccaggtg

SEQ ID NO: 775 - DNA, 17 bp, Homo sapiens
attggtgtaccaggtgc

SEQ ID NO: 776 - DNA, 17 bp, Homo sapiens
ttggtgtaccaggtgcc

SEQ ID NO: 777 - DNA, 17 bp, Homo sapiens
tggtgtaccaggtgcca

SEQ ID NO: 778 - DNA, 17 bp, Homo sapiens
ggtgtaccaggtgccac

SEQ ID NO: 779 - DNA, 17 bp, Homo sapiens
gtgtaccaggtgccaca

SEQ ID NO: 780 - DNA, 17 bp, Homo sapiens
tgtaccaggtgccacag

SEQ ID NO: 781 - DNA, 17 bp, Homo sapiens
gtaccaggtgccacaga

SEQ ID NO: 782 - DNA, 17 bp, Homo sapiens
taccaggtgccacagag

SEQ ID NO: 783 - DNA, 17 bp, Homo sapiens
accaggtgccacagagc

SEQ ID NO: 784 - DNA, 17 bp, Homo sapiens
ccaggtgccacagagca

SEQ ID NO: 785 - DNA, 17 bp, Homo sapiens
caggtgccacagagcac

SEQ ID NO: 786 - DNA, 17 bp, Homo sapiens
aggtgccacagagcaca

SEQ ID NO: 787 - DNA, 17 bp, Homo sapiens
ggtgccacagagcacac

SEQ ID NO: 788 - DNA, 17 bp, Homo sapiens
gtgccacagagcacaca

SEQ ID NO: 789 - DNA, 17 bp, Homo sapiens
tgccacagagcacacaa

SEQ ID NO: 790 - DNA, 17 bp, Homo sapiens
gccacagagcacacaag

SEQ ID NO: 791 - DNA, 17 bp, Homo sapiens
ccacagagcacacaaga

SEQ ID NO: 792 - DNA, 17 bp, Homo sapiens
cacagagcacacaagaa

SEQ ID NO: 793 - DNA, 17 bp, Homo sapiens
acagagcacacaagaag

SEQ ID NO: 794 - DNA, 17 bp, Homo sapiens
cagagcacacaagaagt

SEQ ID NO: 795 - DNA, 17 bp, Homo sapiens
agagcacacaagaagta

SEQ ID NO: 796 - DNA, 17 bp, Homo sapiens
gagcacacaagaagtat

SEQ ID NO: 797 - DNA, 17 bp, Homo sapiens
agcacacaagaagtatc

SEQ ID NO: 798 - DNA, 17 bp, Homo sapiens
gcacacaagaagtatca

SEQ ID NO: 799 - DNA, 17 bp, Homo sapiens
cacacaagaagtatcag

SEQ ID NO: 800 - DNA, 17 bp, Homo sapiens
acacaagaagtatcagg

SEQ ID NO: 801 - DNA, 17 bp, Homo sapiens
cacaagaagtatcagga

SEQ ID NO: 802 - DNA, 17 bp, Homo sapiens
acaagaagtatcaggag

SEQ ID NO: 803 - DNA, 17 bp, Homo sapiens
caagaagtatcaggagc

SEQ ID NO: 804 - DNA, 17 bp, Homo sapiens
aagaagtatcaggagca

SEQ ID NO: 805 - DNA, 17 bp, Homo sapiens
agaagtatcaggagcag

SEQ ID NO: 806 - DNA, 17 bp, Homo sapiens
gaagtatcaggagcagg

SEQ ID NO: 807 - DNA, 17 bp, Homo sapiens
aagtatcaggagcagga

SEQ ID NO: 808 - DNA, 17 bp, Homo sapiens
agtatcaggagcaggaa

SEQ ID NO: 809 - DNA, 17 bp, Homo sapiens
gtatcaggagcaggaag

SEQ ID NO: 810 - DNA, 17 bp, Homo sapiens
tatcaggagcaggaagg

SEQ ID NO: 811 - DNA, 17 bp, Homo sapiens
atcaggagcaggaaggg

SEQ ID NO: 812 - DNA, 17 bp, Homo sapiens
tcaggagcaggaaggga

SEQ ID NO: 813 - DNA, 17 bp, Homo sapiens
caggagcaggaagggat

SEQ ID NO: 814 - DNA, 17 bp, Homo sapiens
aggagcaggaagggatg

SEQ ID NO: 815 - DNA, 17 bp, Homo sapiens
ggagcaggaagggatgg

SEQ ID NO: 816 - DNA, 17 bp, Homo sapiens
gagcaggaagggatggg

SEQ ID NO: 817 - DNA, 17 bp, Homo sapiens
agcaggaagggatgggg

SEQ ID NO: 818 - DNA, 17 bp, Homo sapiens
gcaggaagggatgggga

SEQ ID NO: 819 - DNA, 17 bp, Homo sapiens
caggaagggatggggaa

SEQ ID NO: 820 - DNA, 17 bp, Homo sapiens
aggaagggatggggaat

SEQ ID NO: 821 - DNA, 17 bp, Homo sapiens
ggaagggatggggaatg

SEQ ID NO: 822 - DNA, 17 bp, Homo sapiens
gaagggatggggaatgt

SEQ ID NO: 823 - DNA, 17 bp, Homo sapiens
aagggatggggaatgtg

SEQ ID NO: 824 - DNA, 17 bp, Homo sapiens
agggatggggaatgtga

SEQ ID NO: 825 - DNA, 17 bp, Homo sapiens
gggatggggaatgtgat

SEQ ID NO: 826 - DNA, 17 bp, Homo sapiens
ggatggggaatgtgatg

SEQ ID NO: 827 - DNA, 17 bp, Homo sapiens
gatggggaatgtgatgt

SEQ ID NO: 828 - DNA, 17 bp, Homo sapiens
atggggaatgtgatgtt

SEQ ID NO: 829 - DNA, 17 bp, Homo sapiens
tggggaatgtgatgttt

SEQ ID NO: 830 - DNA, 17 bp, Homo sapiens
ggggaatgtgatgtttt

SEQ ID NO: 831 - DNA, 17 bp, Homo sapiens
gggaatgtgatgttttt

SEQ ID NO: 832 - DNA, 17 bp, Homo sapiens
ggaatgtgatgttttta

SEQ ID NO: 833 - DNA, 17 bp, Homo sapiens
gaatgtgatgtttttaa

SEQ ID NO: 834 - DNA, 17 bp, Homo sapiens
aatgtgatgtttttaaa

SEQ ID NO: 835 - DNA, 17 bp, Homo sapiens
atgtgatgtttttaaag

SEQ ID NO: 836 - DNA, 17 bp, Homo sapiens
tgtgatgtttttaaaga

SEQ ID NO: 837 - DNA, 17 bp, Homo sapiens
gtgatgtttttaaagaa

SEQ ID NO: 838 - DNA, 17 bp, Homo sapiens
tgatgtttttaaagaaa

SEQ ID NO: 839 - DNA, 17 bp, Homo sapiens
gatgtttttaaagaaat

SEQ ID NO: 840 - DNA, 17 bp, Homo sapiens
atgtttttaaagaaatc

SEQ ID NO: 841 - DNA, 17 bp, Homo sapiens
tgtttttaaagaaatcc

SEQ ID NO: 842 - DNA, 17 bp, Homo sapiens
gtttttaaagaaatcct

SEQ ID NO: 843 - DNA, 17 bp, Homo sapiens
tttttaaagaaatcctt

SEQ ID NO: 844 - DNA, 17 bp, Homo sapiens
ttttaaagaaatccttt

SEQ ID NO: 845 - DNA, 17 bp, Homo sapiens
tttaaagaaatcctttg

SEQ ID NO: 846 - DNA, 17 bp, Homo sapiens
ttaaagaaatcctttga

SEQ ID NO: 847 - DNA, 17 bp, Homo sapiens
taaagaaatcctttgaa

SEQ ID NO: 848 - DNA, 17 bp, Homo sapiens
aaagaaatcctttgaag

SEQ ID NO: 849 - DNA, 17 bp, Homo sapiens
aagaaatcctttgaaga

SEQ ID NO: 850 - DNA, 17 bp, Homo sapiens
agaaatcctttgaagat

SEQ ID NO: 851 - DNA, 17 bp, Homo sapiens
gaaatcctttgaagatg

SEQ ID NO: 852 - DNA, 17 bp, Homo sapiens
aaatcctttgaagatga

SEQ ID NO: 853 - DNA, 17 bp, Homo sapiens
aatcctttgaagatgat

SEQ ID NO: 854 - DNA, 17 bp, Homo sapiens
atcctttgaagatgatg

SEQ ID NO: 855 - DNA, 17 bp, Homo sapiens
tcctttgaagatgatgc

SEQ ID NO: 856 - DNA, 17 bp, Homo sapiens
cctttgaagatgatgct

SEQ ID NO: 857 - DNA, 17 bp, Homo sapiens
ctttgaagatgatgctg

SEQ ID NO: 858 - DNA, 17 bp, Homo sapiens
tttgaagatgatgctgc

SEQ ID NO: 859 - DNA, 17 bp, Homo sapiens
ttgaagatgatgctgct

SEQ ID NO: 860 - DNA, 17 bp, Homo sapiens
tgaagatgatgctgctt

SEQ ID NO: 861 - DNA, 17 bp, Homo sapiens
gaagatgatgctgcttt

SEQ ID NO: 862 - DNA, 25 bp, Homo sapiens
caacttcagttggtcagccctccac

SEQ ID NO: 863 - DNA, 25 bp, Homo sapiens
aacttcagttggtcagccctccaca

SEQ ID NO: 864 - DNA, 25 bp, Homo sapiens
acttcagttggtcagccctccacat

SEQ ID NO: 865 - DNA, 25 bp, Homo sapiens
cttcagttggtcagccctccacatc

SEQ ID NO: 866 - DNA, 25 bp, Homo sapiens
ttcagttggtcagccctccacatcc

SEQ ID NO: 867 - DNA, 25 bp, Homo sapiens
tcagttggtcagccctccacatcca

SEQ ID NO: 868 - DNA, 25 bp, Homo sapiens
cagttggtcagccctccacatccac

SEQ ID NO: 869 - DNA, 25 bp, Homo sapiens
agttggtcagccctccacatccact

SEQ ID NO: 870 - DNA, 25 bp, Homo sapiens
gttggtcagccctccacatccactt

SEQ ID NO: 871 - DNA, 25 bp, Homo sapiens
ttggtcagccctccacatccacttt

SEQ ID NO: 872 - DNA, 25 bp, Homo sapiens
tggtcagccctccacatccactttc

SEQ ID NO: 873 - DNA, 25 bp, Homo sapiens
ggtcagccctccacatccactttca

SEQ ID NO: 874 - DNA, 25 bp, Homo sapiens
gtcagccctccacatccactttcaa

SEQ ID NO: 875 - DNA, 25 bp, Homo sapiens
tcagccctccacatccactttcaag

SEQ ID NO: 876 - DNA, 25 bp, Homo sapiens
cagccctccacatccactttcaagg

SEQ ID NO: 877 - DNA, 25 bp, Homo sapiens
agccctccacatccactttcaaggc

SEQ ID NO: 878 - DNA, 25 bp, Homo sapiens
gccctccacatccactttcaaggct

SEQ ID NO: 879 - DNA, 25 bp, Homo sapiens
ccctccacatccactttcaaggcta

SEQ ID NO: 880 - DNA, 25 bp, Homo sapiens
cctccacatccactttcaaggctac

SEQ ID NO: 881 - DNA, 25 bp, Homo sapiens
ctccacatccactttcaaggctacg

SEQ ID NO: 882 - DNA, 25 bp, Homo sapiens
tccacatccactttcaaggctacgg

SEQ ID NO: 883 - DNA, 25 bp, Homo sapiens
ccacatccactttcaaggctacggg

SEQ ID NO: 884 - DNA, 25 bp, Homo sapiens
cacatccactttcaaggctacgggg

SEQ ID NO: 885 - DNA, 25 bp, Homo sapiens
acatccactttcaaggctacgggga

SEQ ID NO: 886 - DNA, 25 bp, Homo sapiens
catccactttcaaggctacggggaa

SEQ ID NO: 887 - DNA, 25 bp, Homo sapiens
atccactttcaaggctacggggaac

SEQ ID NO: 888 - DNA, 25 bp, Homo sapiens
tccactttcaaggctacggggaacc

SEQ ID NO: 889 - DNA, 25 bp, Homo sapiens
ccactttcaaggctacggggaacca

SEQ ID NO: 890 - DNA, 25 bp, Homo sapiens
cactttcaaggctacggggaaccaa

SEQ ID NO: 891 - DNA, 25 bp, Homo sapiens
actttcaaggctacggggaaccaac

SEQ ID NO: 892 - DNA, 25 bp, Homo sapiens
ctttcaaggctacggggaaccaacc

SEQ ID NO: 893 - DNA, 25 bp, Homo sapiens
tttcaaggctacggggaaccaacct

SEQ ID NO: 894 - DNA, 25 bp, Homo sapiens
ttcaaggctacggggaaccaacctc

SEQ ID NO: 895 - DNA, 25 bp, Homo sapiens
tcaaggctacggggaaccaacctcc

SEQ ID NO: 896 - DNA, 25 bp, Homo sapiens
caaggctacggggaaccaacctccc

SEQ ID NO: 897 - DNA, 25 bp, Homo sapiens
aaggctacggggaaccaacctcccc

SEQ ID NO: 898 - DNA, 25 bp, Homo sapiens
aggctacggggaaccaacctccccc

SEQ ID NO: 899 - DNA, 25 bp, Homo sapiens
ggctacggggaaccaacctccccca

SEQ ID NO: 900 - DNA, 25 bp, Homo sapiens
gctacggggaaccaacctcccccac

SEQ ID NO: 901 - DNA, 25 bp, Homo sapiens
ctacggggaaccaacctcccccact

SEQ ID NO: 902 - DNA, 25 bp, Homo sapiens
tacggggaaccaacctcccccacta

SEQ ID NO: 903 - DNA, 25 bp, Homo sapiens
acggggaaccaacctcccccactag

SEQ ID NO: 904 - DNA, 25 bp, Homo sapiens
cggggaaccaacctcccccactagt

SEQ ID NO: 905 - DNA, 25 bp, Homo sapiens
ggggaaccaacctcccccactagtg

SEQ ID NO: 906 - DNA, 25 bp, Homo sapiens
gggaaccaacctcccccactagtgg

SEQ ID NO: 907 - DNA, 25 bp, Homo sapiens
ggaaccaacctcccccactagtggg

SEQ ID NO: 908 - DNA, 25 bp, Homo sapiens
gaaccaacctcccccactagtggga

SEQ ID NO: 909 - DNA, 25 bp, Homo sapiens
aaccaacctcccccactagtgggaa

SEQ ID NO: 910 - DNA, 25 bp, Homo sapiens
accaacctcccccactagtgggaac

SEQ ID NO: 911 - DNA, 25 bp, Homo sapiens
ccaacctcccccactagtgggaact

SEQ ID NO: 912 - DNA, 25 bp, Homo sapiens
caacctcccccactagtgggaactt

SEQ ID NO: 913 - DNA, 25 bp, Homo sapiens
aacctcccccactagtgggaactta

SEQ ID NO: 914 - DNA, 25 bp, Homo sapiens
acctcccccactagtgggaacttac

SEQ ID NO: 915 - DNA, 25 bp, Homo sapiens
cctcccccactagtgggaacttaca

SEQ ID NO: 916 - DNA, 25 bp, Homo sapiens
ctcccccactagtgggaacttacaa

SEQ ID NO: 917 - DNA, 25 bp, Homo sapiens
tcccccactagtgggaacttacaat

SEQ ID NO: 918 - DNA, 25 bp, Homo sapiens
cccccactagtgggaacttacaata

SEQ ID NO: 919 - DNA, 25 bp, Homo sapiens
ccccactagtgggaacttacaatac

SEQ ID NO: 920 - DNA, 25 bp, Homo sapiens
cccactagtgggaacttacaataca

SEQ ID NO: 921 - DNA, 25 bp, Homo sapiens
ccactagtgggaacttacaatacac

SEQ ID NO: 922 - DNA, 25 bp, Homo sapiens
cactagtgggaacttacaatacact

SEQ ID NO: 923 - DNA, 25 bp, Homo sapiens
actagtgggaacttacaatacactt

SEQ ID NO: 924 - DNA, 25 bp, Homo sapiens
ctagtgggaacttacaatacacttc

SEQ ID NO: 925 - DNA, 25 bp, Homo sapiens
tagtgggaacttacaatacacttct

SEQ ID NO: 926 - DNA, 25 bp, Homo sapiens
agtgggaacttacaatacacttctc

SEQ ID NO: 927 - DNA, 25 bp, Homo sapiens
gtgggaacttacaatacacttctct

SEQ ID NO: 928 - DNA, 25 bp, Homo sapiens
tgggaacttacaatacacttctctc

SEQ ID NO: 929 - DNA, 25 bp, Homo sapiens
gggaacttacaatacacttctctcc

SEQ ID NO: 930 - DNA, 25 bp, Homo sapiens
ggaacttacaatacacttctctcca

SEQ ID NO: 931 - DNA, 25 bp, Homo sapiens
gaacttacaatacacttctctccag

SEQ ID NO: 932 - DNA, 25 bp, Homo sapiens
aacttacaatacacttctctccagg

SEQ ID NO: 933 - DNA, 25 bp, Homo sapiens
acttacaatacacttctctccagga

SEQ ID NO: 934 - DNA, 25 bp, Homo sapiens
cttacaatacacttctctccaggac

SEQ ID NO: 935 - DNA, 25 bp, Homo sapiens
ttacaatacacttctctccaggact

SEQ ID NO: 936 - DNA, 25 bp, Homo sapiens
tacaatacacttctctccaggactg

SEQ ID NO: 937 - DNA, 25 bp, Homo sapiens
acaatacacttctctccaggactga

SEQ ID NO: 938 - DNA, 25 bp, Homo sapiens
caatacacttctctccaggactgac

SEQ ID NO: 939 - DNA, 25 bp, Homo sapiens
aatacacttctctccaggactgaca

SEQ ID NO: 940 - DNA, 25 bp, Homo sapiens
atacacttctctccaggactgacag

SEQ ID NO: 941 - DNA, 25 bp, Homo sapiens
tacacttctctccaggactgacagc

SEQ ID NO: 942 - DNA, 25 bp, Homo sapiens
acacttctctccaggactgacagct

SEQ ID NO: 943 - DNA, 25 bp, Homo sapiens
cacttctctccaggactgacagctg

SEQ ID NO: 944 - DNA, 25 bp, Homo sapiens
acttctctccaggactgacagctgc

SEQ ID NO: 945 - DNA, 25 bp, Homo sapiens
cttctctccaggactgacagctgct

SEQ ID NO: 946 - DNA, 25 bp, Homo sapiens
ttctctccaggactgacagctgctc

SEQ ID NO: 947 - DNA, 25 bp, Homo sapiens
tctctccaggactgacagctgctcc

SEQ ID NO: 948 - DNA, 25 bp, Homo sapiens
ctctccaggactgacagctgctcct

SEQ ID NO: 949 - DNA, 25 bp, Homo sapiens
tctccaggactgacagctgctcctc

SEQ ID NO: 950 - DNA, 25 bp, Homo sapiens
ctccaggactgacagctgctcctca

SEQ ID NO: 951 - DNA, 25 bp, Homo sapiens
tccaggactgacagctgctcctcag

SEQ ID NO: 952 - DNA, 25 bp, Homo sapiens
ccaggactgacagctgctcctcagc

SEQ ID NO: 953 - DNA, 25 bp, Homo sapiens
caggactgacagctgctcctcagcc

SEQ ID NO: 954 - DNA, 25 bp, Homo sapiens
aggactgacagctgctcctcagccc

SEQ ID NO: 955 - DNA, 25 bp, Homo sapiens
ggactgacagctgctcctcagccca

SEQ ID NO: 956 - DNA, 25 bp, Homo sapiens
gactgacagctgctcctcagcccag

SEQ ID NO: 957 - DNA, 25 bp, Homo sapiens
actgacagctgctcctcagcccagg

SEQ ID NO: 958 - DNA, 25 bp, Homo sapiens
ctgacagctgctcctcagcccaggc

SEQ ID NO: 959 - DNA, 25 bp, Homo sapiens
tgacagctgctcctcagcccaggcc

SEQ ID NO: 960 - DNA, 25 bp, Homo sapiens
gacagctgctcctcagcccaggccc

SEQ ID NO: 961 - DNA, 25 bp, Homo sapiens
acagctgctcctcagcccaggccca

SEQ ID NO: 962 - DNA, 25 bp, Homo sapiens
cagctgctcctcagcccaggcccag

SEQ ID NO: 963 - DNA, 25 bp, Homo sapiens
agctgctcctcagcccaggcccagt

SEQ ID NO: 964 - DNA, 25 bp, Homo sapiens
gctgctcctcagcccaggcccagta

SEQ ID NO: 965 - DNA, 25 bp, Homo sapiens
ctgctcctcagcccaggcccagtat

SEQ ID NO: 966 - DNA, 25 bp, Homo sapiens
tgctcctcagcccaggcccagtatg

SEQ ID NO: 967 - DNA, 25 bp, Homo sapiens
gctcctcagcccaggcccagtatga

SEQ ID NO: 968 - DNA, 25 bp, Homo sapiens
ctcctcagcccaggcccagtatgat

SEQ ID NO: 969 - DNA, 25 bp, Homo sapiens
tcctcagcccaggcccagtatgata

SEQ ID NO: 970 - DNA, 25 bp, Homo sapiens
cctcagcccaggcccagtatgatac

SEQ ID NO: 971 - DNA, 25 bp, Homo sapiens
ctcagcccaggcccagtatgatacc

SEQ ID NO: 972 - DNA, 25 bp, Homo sapiens
tcagcccaggcccagtatgataccc

SEQ ID NO: 973 - DNA, 25 bp, Homo sapiens
cagcccaggcccagtatgatacccc

SEQ ID NO: 974 - DNA, 25 bp, Homo sapiens
agcccaggcccagtatgataccccg

SEQ ID NO: 975 - DNA, 25 bp, Homo sapiens
gcccaggcccagtatgataccccga

SEQ ID NO: 976 - DNA, 25 bp, Homo sapiens
cccaggcccagtatgataccccgaa

SEQ ID NO: 977 - DNA, 25 bp, Homo sapiens
ccaggcccagtatgataccccgaaa

SEQ ID NO: 978 - DNA, 25 bp, Homo sapiens
caggcccagtatgataccccgaaag

SEQ ID NO: 979 - DNA, 25 bp, Homo sapiens
aggcccagtatgataccccgaaagc

SEQ ID NO: 980 - DNA, 25 bp, Homo sapiens
ggcccagtatgataccccgaaagct

SEQ ID NO: 981 - DNA, 25 bp, Homo sapiens
gcccagtatgataccccgaaagctg

SEQ ID NO: 982 - DNA, 25 bp, Homo sapiens
cccagtatgataccccgaaagctgg

SEQ ID NO: 983 - DNA, 25 bp, Homo sapiens
ccagtatgataccccgaaagctggg

SEQ ID NO: 984 - DNA, 25 bp, Homo sapiens
cagtatgataccccgaaagctggga

SEQ ID NO: 985 - DNA, 25 bp, Homo sapiens
agtatgataccccgaaagctgggaa

SEQ ID NO: 986 - DNA, 25 bp, Homo sapiens
gtatgataccccgaaagctgggaag

SEQ ID NO: 987 - DNA, 25 bp, Homo sapiens
tatgataccccgaaagctgggaagc

SEQ ID NO: 988 - DNA, 25 bp, Homo sapiens
atgataccccgaaagctgggaagcc

SEQ ID NO: 989 - DNA, 25 bp, Homo sapiens
tgataccccgaaagctgggaagcca

SEQ ID NO: 990 - DNA, 25 bp, Homo sapiens
gataccccgaaagctgggaagccag

SEQ ID NO: 991 - DNA, 25 bp, Homo sapiens
ataccccgaaagctgggaagccagg

SEQ ID NO: 992 - DNA, 25 bp, Homo sapiens
taccccgaaagctgggaagccaggt

SEQ ID NO: 993 - DNA, 25 bp, Homo sapiens
accccgaaagctgggaagccaggtc

SEQ ID NO: 994 - DNA, 25 bp, Homo sapiens
ccccgaaagctgggaagccaggtct

SEQ ID NO: 995 - DNA, 25 bp, Homo sapiens
cccgaaagctgggaagccaggtcta

SEQ ID NO: 996 - DNA, 25 bp, Homo sapiens
ccgaaagctgggaagccaggtctac

SEQ ID NO: 997 - DNA, 25 bp, Homo sapiens
cgaaagctgggaagccaggtctacc

SEQ ID NO: 998 - DNA, 25 bp, Homo sapiens
gaaagctgggaagccaggtctacct

SEQ ID NO: 999 - DNA, 25 bp, Homo sapiens
aaagctgggaagccaggtctacctg

SEQ ID NO: 1000 - DNA, 25 bp, Homo sapiens
aagctgggaagccaggtctacctgc

SEQ ID NO: 1001 - DNA, 25 bp, Homo sapiens
agctgggaagccaggtctacctgcc

SEQ ID NO: 1002 - DNA, 25 bp, Homo sapiens
gctgggaagccaggtctacctgccc

SEQ ID NO: 1003 - DNA, 25 bp, Homo sapiens
ctgggaagccaggtctacctgcccc

SEQ ID NO: 1004 - DNA, 25 bp, Homo sapiens
tgggaagccaggtctacctgcccca

SEQ ID NO: 1005 - DNA, 25 bp, Homo sapiens
gggaagccaggtctacctgccccag

SEQ ID NO: 1006 - DNA, 25 bp, Homo sapiens
ggaagccaggtctacctgccccaga

SEQ ID NO: 1007 - DNA, 25 bp, Homo sapiens
gaagccaggtctacctgccccagac

SEQ ID NO: 1008 - DNA, 25 bp, Homo sapiens
aagccaggtctacctgccccagacg

SEQ ID NO: 1009 - DNA, 25 bp, Homo sapiens
agccaggtctacctgccccagacga

SEQ ID NO: 1010 - DNA, 25 bp, Homo sapiens
gccaggtctacctgccccagacgaa

SEQ ID NO: 1011 - DNA, 25 bp, Homo sapiens
ccaggtctacctgccccagacgaat

SEQ ID NO: 1012 - DNA, 25 bp, Homo sapiens
caggtctacctgccccagacgaatt

SEQ ID NO: 1013 - DNA, 25 bp, Homo sapiens
aggtctacctgccccagacgaattg

SEQ ID NO: 1014 - DNA, 25 bp, Homo sapiens
ggtctacctgccccagacgaattgg

SEQ ID NO: 1015 - DNA, 25 bp, Homo sapiens
gtctacctgccccagacgaattggt

SEQ ID NO: 1016 - DNA, 25 bp, Homo sapiens
tctacctgccccagacgaattggtg

SEQ ID NO: 1017 - DNA, 25 bp, Homo sapiens
ctacctgccccagacgaattggtgt

SEQ ID NO: 1018 - DNA, 25 bp, Homo sapiens
tacctgccccagacgaattggtgta

SEQ ID NO: 1019 - DNA, 25 bp, Homo sapiens
acctgccccagacgaattggtgtac

SEQ ID NO: 1020 - DNA, 25 bp, Homo sapiens
cctgccccagacgaattggtgtacc

SEQ ID NO: 1021 - DNA, 25 bp, Homo sapiens
ctgccccagacgaattggtgtacca

SEQ ID NO: 1022 - DNA, 25 bp, Homo sapiens
tgccccagacgaattggtgtaccag

SEQ ID NO: 1023 - DNA, 25 bp, Homo sapiens
gccccagacgaattggtgtaccagg

SEQ ID NO: 1024 - DNA, 25 bp, Homo sapiens
ccccagacgaattggtgtaccaggt

SEQ ID NO: 1025 - DNA, 25 bp, Homo sapiens
cccagacgaattggtgtaccaggtg

SEQ ID NO: 1026 - DNA, 25 bp, Homo sapiens
ccagacgaattggtgtaccaggtgc

SEQ ID NO: 1027 - DNA, 25 bp, Homo sapiens
cagacgaattggtgtaccaggtgcc

SEQ ID NO: 1028 - DNA, 25 bp, Homo sapiens
agacgaattggtgtaccaggtgcca

SEQ ID NO: 1029 - DNA, 25 bp, Homo sapiens
gacgaattggtgtaccaggtgccac

SEQ ID NO: 1030 - DNA, 25 bp, Homo sapiens
acgaattggtgtaccaggtgccaca

SEQ ID NO: 1031 - DNA, 25 bp, Homo sapiens
cgaattggtgtaccaggtgccacag

SEQ ID NO: 1032 - DNA, 25 bp, Homo sapiens
gaattggtgtaccaggtgccacaga

SEQ ID NO: 1033 - DNA, 25 bp, Homo sapiens
aattggtgtaccaggtgccacagag

SEQ ID NO: 1034 - DNA, 25 bp, Homo sapiens
attggtgtaccaggtgccacagagc

SEQ ID NO: 1035 - DNA, 25 bp, Homo sapiens
ttggtgtaccaggtgccacagagca

SEQ ID NO: 1036 - DNA, 25 bp, Homo sapiens
tggtgtaccaggtgccacagagcac

SEQ ID NO: 1037 - DNA, 25 bp, Homo sapiens
ggtgtaccaggtgccacagagcaca

SEQ ID NO: 1038 - DNA, 25 bp, Homo sapiens
gtgtaccaggtgccacagagcacac

SEQ ID NO: 1039 - DNA, 25 bp, Homo sapiens
tgtaccaggtgccacagagcacaca

SEQ ID NO: 1040 - DNA, 25 bp, Homo sapiens
gtaccaggtgccacagagcacacaa

SEQ ID NO: 1041 - DNA, 25 bp, Homo sapiens
taccaggtgccacagagcacacaag

SEQ ID NO: 1042 - DNA, 25 bp, Homo sapiens
accaggtgccacagagcacacaaga

SEQ ID NO: 1043 - DNA, 25 bp, Homo sapiens
ccaggtgccacagagcacacaagaa

SEQ ID NO: 1044 - DNA, 25 bp, Homo sapiens
caggtgccacagagcacacaagaag

SEQ ID NO: 1045 - DNA, 25 bp, Homo sapiens
aggtgccacagagcacacaagaagt

SEQ ID NO: 1046 - DNA, 25 bp, Homo sapiens
ggtgccacagagcacacaagaagta

SEQ ID NO: 1047 - DNA, 25 bp, Homo sapiens
gtgccacagagcacacaagaagtat

SEQ ID NO: 1048 - DNA, 25 bp, Homo sapiens
tgccacagagcacacaagaagtatc

SEQ ID NO: 1049 - DNA, 25 bp, Homo sapiens
gccacagagcacacaagaagtatca

SEQ ID NO: 1050 - DNA, 25 bp, Homo sapiens
ccacagagcacacaagaagtatcag

SEQ ID NO: 1051 - DNA, 25 bp, Homo sapiens
cacagagcacacaagaagtatcagg

SEQ ID NO: 1052 - DNA, 25 bp, Homo sapiens
acagagcacacaagaagtatcagga

SEQ ID NO: 1053 - DNA, 25 bp, Homo sapiens
cagagcacacaagaagtatcaggag

SEQ ID NO: 1054 - DNA, 25 bp, Homo sapiens
agagcacacaagaagtatcaggagc

SEQ ID NO: 1055 - DNA, 25 bp, Homo sapiens
gagcacacaagaagtatcaggagca

SEQ ID NO: 1056 - DNA, 25 bp, Homo sapiens
agcacacaagaagtatcaggagcag

SEQ ID NO: 1057 - DNA, 25 bp, Homo sapiens
gcacacaagaagtatcaggagcagg

SEQ ID NO: 1058 - DNA, 25 bp, Homo sapiens
cacacaagaagtatcaggagcagga

SEQ ID NO: 1059 - DNA, 25 bp, Homo sapiens
acacaagaagtatcaggagcaggaa

SEQ ID NO: 1060 - DNA, 25 bp, Homo sapiens
cacaagaagtatcaggagcaggaag

SEQ ID NO: 1061 - DNA, 25 bp, Homo sapiens
acaagaagtatcaggagcaggaagg

SEQ ID NO: 1062 - DNA, 25 bp, Homo sapiens
caagaagtatcaggagcaggaaggg

SEQ ID NO: 1063 - DNA, 25 bp, Homo sapiens
aagaagtatcaggagcaggaaggga

SEQ ID NO: 1064 - DNA, 25 bp, Homo sapiens
agaagtatcaggagcaggaagggat

SEQ ID NO: 1065 - DNA, 25 bp, Homo sapiens
gaagtatcaggagcaggaagggatg

SEQ ID NO: 1066 - DNA, 25 bp, Homo sapiens
aagtatcaggagcaggaagggatgg

SEQ ID NO: 1067 - DNA, 25 bp, Homo sapiens
agtatcaggagcaggaagggatggg

SEQ ID NO: 1068 - DNA, 25 bp, Homo sapiens
gtatcaggagcaggaagggatgggg

SEQ ID NO: 1069 - DNA, 25 bp, Homo sapiens
tatcaggagcaggaagggatgggga

SEQ ID NO: 1070 - DNA, 25 bp, Homo sapiens
atcaggagcaggaagggatggggaa

SEQ ID NO: 1071 - DNA, 25 bp, Homo sapiens
tcaggagcaggaagggatggggaat

SEQ ID NO: 1072 - DNA, 25 bp, Homo sapiens
caggagcaggaagggatggggaatg

SEQ ID NO: 1073 - DNA, 25 bp, Homo sapiens
aggagcaggaagggatggggaatgt

SEQ ID NO: 1074 - DNA, 25 bp, Homo sapiens
ggagcaggaagggatggggaatgtg

SEQ ID NO: 1075 - DNA, 25 bp, Homo sapiens
gagcaggaagggatggggaatgtga

SEQ ID NO: 1076 - DNA, 25 bp, Homo sapiens
agcaggaagggatggggaatgtgat

SEQ ID NO: 1077 - DNA, 25 bp, Homo sapiens
gcaggaagggatggggaatgtgatg

SEQ ID NO: 1078 - DNA, 25 bp, Homo sapiens
caggaagggatggggaatgtgatgt

SEQ ID NO: 1079 - DNA, 25 bp, Homo sapiens
aggaagggatggggaatgtgatgtt

SEQ ID NO: 1080 - DNA, 25 bp, Homo sapiens
ggaagggatggggaatgtgatgttt

SEQ ID NO: 1081 - DNA, 25 bp, Homo sapiens
gaagggatggggaatgtgatgtttt

SEQ ID NO: 1082 - DNA, 25 bp, Homo sapiens
aagggatggggaatgtgatgttttt

SEQ ID NO: 1083 - DNA, 25 bp, Homo sapiens
agggatggggaatgtgatgttttta

SEQ ID NO: 1084 - DNA, 25 bp, Homo sapiens
gggatggggaatgtgatgtttttaa

SEQ ID NO: 1085 - DNA, 25 bp, Homo sapiens
ggatggggaatgtgatgtttttaaa

SEQ ID NO: 1086 - DNA, 25 bp, Homo sapiens
gatggggaatgtgatgtttttaaag

SEQ ID NO: 1087 - DNA, 25 bp, Homo sapiens
atggggaatgtgatgtttttaaaga

SEQ ID NO: 1088 - DNA, 25 bp, Homo sapiens
tggggaatgtgatgtttttaaagaa

SEQ ID NO: 1089 - DNA, 25 bp, Homo sapiens
ggggaatgtgatgtttttaaagaaa

SEQ ID NO: 1090 - DNA, 25 bp, Homo sapiens
gggaatgtgatgtttttaaagaaat

SEQ ID NO: 1091 - DNA, 25 bp, Homo sapiens
ggaatgtgatgtttttaaagaaatc

SEQ ID NO: 1092 - DNA, 25 bp, Homo sapiens
gaatgtgatgtttttaaagaaatcc

SEQ ID NO: 1093 - DNA, 25 bp, Homo sapiens
aatgtgatgtttttaaagaaatcct

SEQ ID NO: 1094 - DNA, 25 bp, Homo sapiens
atgtgatgtttttaaagaaatcctt

SEQ ID NO: 1095 - DNA, 25 bp, Homo sapiens
tgtgatgtttttaaagaaatccttt

SEQ ID NO: 1096 - DNA, 25 bp, Homo sapiens
gtgatgtttttaaagaaatcctttg

SEQ ID NO: 1097 - DNA, 25 bp, Homo sapiens
tgatgtttttaaagaaatcctttga

SEQ ID NO: 1098 - DNA, 25 bp, Homo sapiens
gatgtttttaaagaaatcctttgaa

SEQ ID NO: 1099 - DNA, 25 bp, Homo sapiens
atgtttttaaagaaatcctttgaag

SEQ ID NO: 1100 - DNA, 25 bp, Homo sapiens
tgtttttaaagaaatcctttgaaga

SEQ ID NO: 1101 - DNA, 25 bp, Homo sapiens
gtttttaaagaaatcctttgaagat

SEQ ID NO: 1102 - DNA, 25 bp, Homo sapiens
tttttaaagaaatcctttgaagatg

SEQ ID NO: 1103 - DNA, 25 bp, Homo sapiens
ttttaaagaaatcctttgaagatga

SEQ ID NO: 1104 - DNA, 25 bp, Homo sapiens
tttaaagaaatcctttgaagatgat

SEQ ID NO: 1105 - DNA, 25 bp, Homo sapiens
ttaaagaaatcctttgaagatgatg

SEQ ID NO: 1106 - DNA, 25 bp, Homo sapiens
taaagaaatcctttgaagatgatgc

SEQ ID NO: 1107 - DNA, 25 bp, Homo sapiens
aaagaaatcctttgaagatgatgct

SEQ ID NO: 1108 - DNA, 25 bp, Homo sapiens
aagaaatcctttgaagatgatgctg

SEQ ID NO: 1109 - DNA, 25 bp, Homo sapiens
agaaatcctttgaagatgatgctgc

SEQ ID NO: 1110 - DNA, 25 bp, Homo sapiens
gaaatcctttgaagatgatgctgct

SEQ ID NO: 1111 - DNA, 25 bp, Homo sapiens
aaatcctttgaagatgatgctgctt

SEQ ID NO: 1112 - DNA, 25 bp, Homo sapiens
aatcctttgaagatgatgctgcttt

SEQ ID NO: 1113 - DNA, 1962 bp, Homo sapiens
atgcctctgttcctcctgctcttacttgtcctgctcctgctgctcgaggacgctggagcccagcaaggcaaatactgtggtctggggttgcaaatgaaccattcaattgaatcaaaaggcaatgaaatcacattgctgttcatgagtggaatccatgtttctggacgcggatttttggcctcatactctgttatagataaacaagatctaattacttgtttggacactgcatccaattttttggaacctgagttcagtaagtactgcccagctggttgtctgcttccttttgctgagatatctggaacaattcctcatggatatagagattcctcgccattgtgcatggctggtgtgcatgcaggagtagtgtcaaacacgttgggcggccaaatcagtgttgtaattagtaaaggtattccctattatgaaagttctttggctaacaacgtcacatctgtggtgggacacttatctacaagtctttttacatttaagacaagtggatgttatggaacactggggatggagtctggtgtgatcgcggatcctcaaataacagcatcatctgtgctggagtggactgaccacacagggcaagagaacagttggaaacccaaaaaagccaggctgaaaaaacctggaccgccttgggctgcttttgccactgatgaataccagtggttacaaatagatttgaataaggaaaagaaaataacaggcattataaccactggatccaccatggtggagcacaattactatgtgtctgcctacagaatcctgtacagtgatgatgggcagaaatggactgtgtacagagagcctggtgtggagcaagataagatatttcaaggaaacaaagattatcaccaggatgtgcgtaataactttttgccaccaattattgcacgttttattagagtgaatcctacccaatggcagcagaaaattgccatgaaaatggagctgctcggatgtcagtttattcctaaaggtcgtcctccaaaacttactcaacctccacctcctcggaacagcaatgacctcaaaaacactacagcccctccaaaaatagccaaaggtcgtgccccaaaatttacgcaaccactacaacctcgcagtagcaatgaatttcctgcacagacagaacaaacaactgccagtcctgatatcagaaatactaccgtaactccaaatgtaaccaaagatgtagcgctggctgcagttcttgtccctgtgctggtcatggtcctcactactctcattctcatattagtgtgtgcttggcactggagaaacagaaagaaaaaaactgaaggcacctatgacttaccttactgggaccgggcaggttggtggaaaggaatgaagcagtttcttcctgcaaaagcagtggaccatgaggaaaccccagttcgctatagcagcagcgaagttaatcacctgagtccaagagaagtcaccacagtgctgcaggctgactctgcagagtatgctcagccactggtaggaggaattgttggtacacttcatcaaagatctacctttaaaccagaagaaggaaaagaagcaggctatgcagacctagatccttacaactcaccagggcaggaagtttatcatgcctatgctgaaccactcccaattacggggcctgagtatgcaaccccaatcatcatggacatgtcagggcaccccacaacttcagttggtcagccctccacatccactttcaaggctacggggaaccaacctcccccactagtgggaacttacaatacacttctctccaggactgacagctgctcctcagcccaggcccagtatgataccccgaaagctgggaagccaggtctacctgccccagacgaattggtgtaccaggtgccacagagcacacaagaagtatcaggagcaggaagggatggggaatgtgatgtttttaaagaaatcctttga

SEQ ID NO: 1114 - protein, 653 aa, Homo sapiens
MPLFLLLLLVLLLLLEDAGAQQGKYCGLGLQMNHSIESKGNEITLLFMSGIHVSGRGFLASYSVIDKQDLITCLDTASNFLEPEFSKYCPAGCLLPFAEISGTIPHGYRDSSPLCMAGVHAGVVSNTLGGQISVVISKGIPYYESSLANNVTSVVGHLSTSLFTFKTSGCYGTLGMESGVIADPQITASSVLEWTDHTGQENSWKPKKARLKKPGPPWAAFATDEYQWLQIDLNKEKKITGIITTGSTMVEHNYYVSAYRILYSDDGQKWTVYREPGVEQDKIFQGNKDYHQDVRNNFLPPIIARFIRVNPTQWQQKIAMKMELLGCQFIPKGRPPKLTQPPPPRNSNDLKNTTAPPKIAKGRAPKFTQPLQPRSSNEFPAQTEQTTASPDIRNTTVTPNVTKDVALAAVLVPVLVMVLTTLILILVCAWHWRNRKKKTEGTYDLPYWDRAGWWKGMKQFLPAKAVDHEETPVRYSSSEVNHLSPREVTTVLQADSAEYAQPLVGGIVGTLHQRSTFKPEEGKEAGYADLDPYNSPGQEVYHAYAEPLPITGPEYATPIIMDMSGHPTTSVGQPSTSTFKATGNQPPPLVGTYNTLLSRTDSCSSAQAQYDTPKAGKPGLPAPDELVYQVPQSTQEVSGAGRDGECDVFKEIL

SEQ ID NO: 1115 - DNA, 60 bp, Homo sapiens
ctgctgctcgaggacgctggagcccagcaaggtgatggatgtggacacactgtactaggc

SEQ ID NO: 1116 - protein, 20 aa, Homo sapiens
LLLEDAGAQQGDGCGHTVLG

SEQ ID NO: 1117 - DNA, 35 bp, Homo sapiens
atggtcctcactactctcattctcatattagtgtg

SEQ ID NO: 1118 - DNA, 33 bp, Homo sapiens
tcaaaggatttctttaaaaacatcacattcccc

SEQ ID NO: 1119 - DNA, 35 bp, Homo sapiens
ctgtactaggccctgagagtggaacccttacatcc

SEQ ID NO: 1120 - DNA, 31 bp, Homo sapiens
ggggtttcctcatggtccactgcttttgcag

SEQ ID NO: 1121 - DNA, 39 bp, Homo sapiens
ccaccatgcctctgttcctcctgctcttacttgtcctgc

SEQ ID NO: 1122 - DNA, 36 bp, Homo sapiens
tcaaaggatttctttaaaaacatcacattccccatc

SEQ ID NO: 1123 - DNA, 33 bp, Homo sapiens
ctgcccggtcccagtaaggtaagtcataggtgc

(19) United States
(12) Patent Application Publication
(10) Pub. No.: US 2003/0032154 A1
(43) Pub. Date: Feb. 13, 2003

(54) Human LCCL domain containing protein

(76) Inventors: Yizhong Gu, Cupertino, CA (US); Cung-Tuong Nguyen, San Jose, CA (US)

Correspondence Address: Stephen G. Ryan, Amersham Biosciences, 800 Centennial Avenue, Piscataway, NJ, 08855, US

(21) Appl. No.: 10/060,830

(22) Filed: Jan. 30, 2002

Related U.S. Application Data
(63) Continuation-in-part of application No. 09/864,761, filed on May 23, 2001.
(60) Provisional application No. 60/325,062, filed on Sep. 25, 2001.

(30) Foreign Application Priority Data
Jan. 30, 2001 (US) PCT/US01/00663
Jan. 30, 2001 (US) PCT/US01/00664
Jan. 30, 2001 (US) PCT/US01/00665
Jan. 30, 2001 (US) PCT/US01/00667
Jan. 30, 2001 (US) PCT/US01/00668
Jan. 30, 2001 (US) PCT/US01/00669

(51) Int. Cl. C12Q 1/68; C07H 21/04; C12N 9/00; C12P 21/02; C12N 5/06

(52) U.S. Cl. 435/183; 435/6; 435/320.1; 435/325; 435/69.1; 536/23.2

(57) ABSTRACT

The invention provides isolated nucleic acids that encode LCP, including two isoforms, and fragments thereof, vectors for propagating and expressing LCP nucleic acids, host cells comprising the nucleic acids and vectors of the present invention, proteins, protein fragments, and protein fusions of the novel LCP isoforms, and antibodies thereto. The invention further provides transgenic cells and non-human organisms comprising human LCP nucleic acids, and transgenic cells and non-human organisms with targeted disruption of the endogenous orthologue of the human LCP gene. The invention further provides pharmaceutical formulations of the nucleic acids, proteins, and antibodies of the present invention, and diagnostic, investigational, and therapeutic methods based on the LCP nucleic acids, proteins, and antibodies of the present invention.

### CROSS REFERENCE TO RELATED APPLICATIONS

This application claims priority under 35 U.S.C. §365(c) to international patent application no. PCT/US01/00663, PCT/US01/00664, PCT/US01/00665, PCT/US01/00667, PCT/US01/00668 and PCT/US01/00669, all filed Jan. 30, 2001; claims priority under 35 U.S.C. §120 to commonly owned and copending U.S. application Serial No. U.S. 09/864,761, filed May 23, 2001; claims priority to U.S. provisional application Serial No. 60/325,062, filed Sep. 25, 2001; the disclosures of which are incorporated herein by reference in their entireties.

### REFERENCE TO SEQUENCE LISTING SUBMITTED ON COMPACT DISC

The present application includes a Sequence Listing filed on one CD-R disc, provided in duplicate, containing a single file named pto_PB0169.txt, having 184 kilobytes, last modified on Jan. 23, 2002 and recorded Jan. 23, 2002. The Sequence Listing contained in said file on said disc is incorporated herein by reference in its entirety.

### FIELD OF THE INVENTION

The present invention relates to a human LCCL domain containing protein, which has two isforms. More specifically, the invention provides isolated nucleic acid molecules encoding human LCCL domain containing protein, fragments thereof, vectors and host cells comprising isolated nucleic acid molecules encoding human LCCL domain containing protein, human LCCL domain containing protein polypeptides, antibodies, transgenic cells and non-human organisms, and diagnostic, therapeutic, and investigational methods of using the same.

### BACKGROUNT OF THE INVENTION

The function of proteins can often be predicted and characterized through the functional domains within the protein sequences. The LCCL domain was first identified in Limulus Factor C, COCH and Lgl 1 genes. Limulus Factor C is an endotoxin-sensitive, trypsin type serine protease, COCH is the candidate gene for the deafness disorder DFNA9 and Lgl 1 is a late gestation lung protein. The LCCL domain is about 93 amino acids in length and contains multiple conserved cysteines (Cys). These three proteins have been grouped together as an LCCL domain containing protein family. DFNA9 is an autosomal dominant, nonsyndromic, progressive sensorineural hearing loss with vestibular pathology. Three point mutations were identified that change conserved amino acid sequence within the LCCL domain of the COCH protein in DFNA9 patients. Robertson et al, Nature Genetics 20:299-302 (1998). The pattern of COCH gene expression in chicken inner ear correlates well with histopathological findings in DFNA9 patients. The high COCH expression in cochlea and vestibule, the only two organ systems identified to be affected in DFNA9 patients, further indicate COCH gene as the candidate gene for DFNA9.

The CUB domain is a 110-amino acid module that was first identified in the complement subcomponents Cls and Clr. Bork FEBS Lett. 282:9-12 (1991). Later, this domain was also identified in bone morphogenetic protein 1 (BMP1), an embryonic sea urchin protein Uegf as well as other proteins. Although there is no clear indication of the function of the CUB domain, it is believed to be involved in the developmental and differentiation processes. Chen et al, J. Biol. Chem. 274:32215-32224 (1999). The Coagulation factor 5/8 C-terminal (FA58C)/discoidin (DSD) domain is a cell surface-attached carbohydrate-binding domain. Members of the discoidin (DSD) domain family, which includes the C1 and C2 repeats of blood coagulation factors V and VIII, occur in a great variety of eukaryotic proteins, most of which have been implicated in cell-adhesion or developmental processes. Baumgartner et al, Protein Sci. 7(7):1626-1631 (1998).

The fluid-mosaic model for the structure of the cell membrane holds that the membrane consists of a protein-embedded bilayer of phospholipids. The proteins embedded within the lipid bilayer can exist in several different positions: extending through both layers, projecting out one side or the other, or totally contained within the bilayer. Jacobson K. et al, Science 268:1441-1442 (1995). The plasma membrane is directly involved in dynamic cellular functions, such as growth, movement, and signaling. Plasma membrane proteins play central roles in such functions. Due to their critical role in cell signaling, membrane proteins are increasingly considered as excellent drug intervention targets. An obvious approach is to develop humanized monoclonal antibodies and use them against membrane targets, which are overexpressed in certain tumors (Mokbel K. and Hassanally D., Curr. Med. Res. Opin. 17:51-59 (2001)).

Recent reports suggest that at least one-third, and likely a higher percentage, of human genes are alternatively spliced. Hanke et al., Trends Genet. 15(1):389-390 (1999); Mironov et al., Genome Res. 9:1288-93 (1999); Brett et al., FEBS Lett. 474(1):83-6 (2000). Alternative splicing has been proposed to account for at least part of the difference between the number of genes recently called from the completed human genome draft sequence—30,000 to 40,000 (Genome International Sequencing Consortium, Nature 409:860-921 (15 February 2001)—and earlier predictions of human gene number that routinely ranged as high as 120,000, Liang et al., Nature Genet. 25(2):239-240 (2000). With the Drosophila homolog of one human gene reported to have 38,000 potential alternatively spliced variants, Schmucker et al., Cell 101:671 (2000), it now appears that alternative splicing may permit the relatively small number of human coding regions to encode millions, perhaps tens of millions, of structurally distinct proteins and protein isoforms.

Due to the critical role of the COCH gene in DFNA9, and the potential role of CUB and DSD/FA58C in cell-cell adhesion and development, there is a need to identify and to characterize new members of the LCCL domain containing gene family as they have potential therapeutic as well as diagnostic roles for neurological and developmental disorders, as well as diseases involving cell-cell adhesion process.

### SUMMARY OF THE INVENTION

The present invention solves these and other needs in the art by providing isolated nucleic acids that encode human LCCL domain containing protein (LCP), which has two isoforms (LCP1 and LCP2), and fragments thereof.

In other aspects, the invention provides vectors for propagating and expressing the nucleic acids of the present invention, host cells comprising the nucleic acids and vectors of the present invention, proteins, protein fragments, and protein fusions of the LCP, and antibodies thereto.

The invention further provides pharmaceutical formulations of the nucleic acids, proteins, and antibodies of the present invention.

In other aspects, the invention provides transgenic cells and non-human organisms comprising LCP nucleic acids, and transgenic cells and non-human organisms with targeted disruption of the endogenous orthologue of the LCP.

The invention additionally provides diagnostic, investigational, and therapeutic methods based on the LCP nucleic acids, proteins, and antibodies of the present invention.

### BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIG. 1(A) schematizes the protein domain structure of LCP1,

FIG. 1(B) presents the alignment of the CUB domain of LCP1 with that of other proteins,

FIG. 1(C) presents the alignment of the LCCL domain of LCP with that of other proteins, and

FIG. 1(D) presents the alignment of the DSD/FA58C domain of LCP with that of other proteins;

FIG. 2 is a map showing the genomic structure of LCP encoded at chromosome 3q12.1;

FIG. 3 presents the nucleotide and predicted amino acid sequences of LCP1;

FIG. 4 presents the nucleotide and predicted amino acid sequences of LCP2; and

FIG. 5 presents the expression profile of LCP by RT-PCR analysis.

### DETAILED DESCRIPTION OF THE INVENTION

Mining the sequence of the human genome for novel human genes, the present inventors have identified LCP, a membrane protein potentially associated with neurological and developmental disorders, as well as diseases involving cell-cell adhesion process.

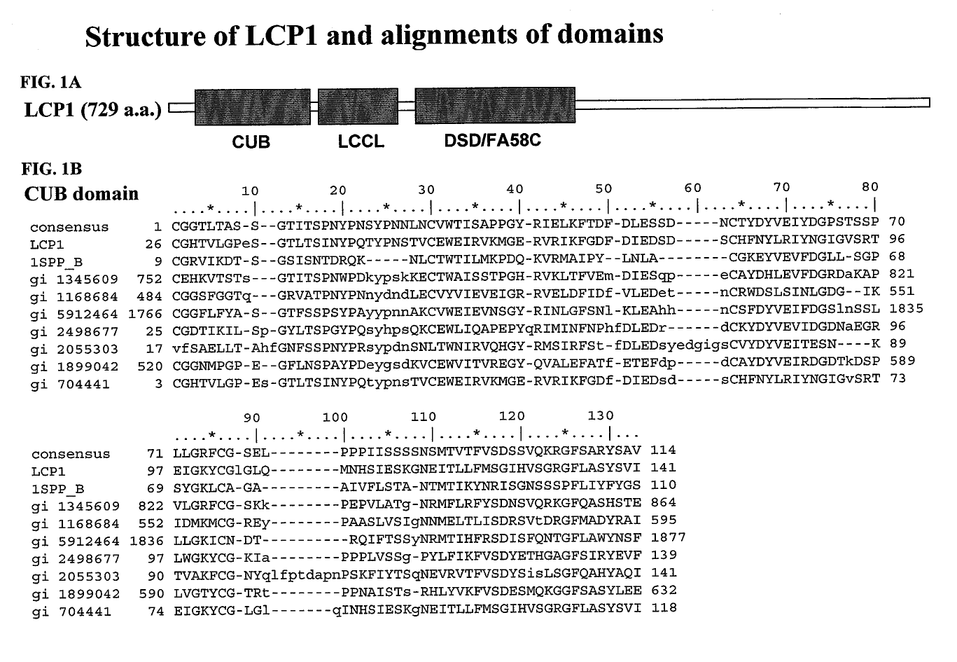

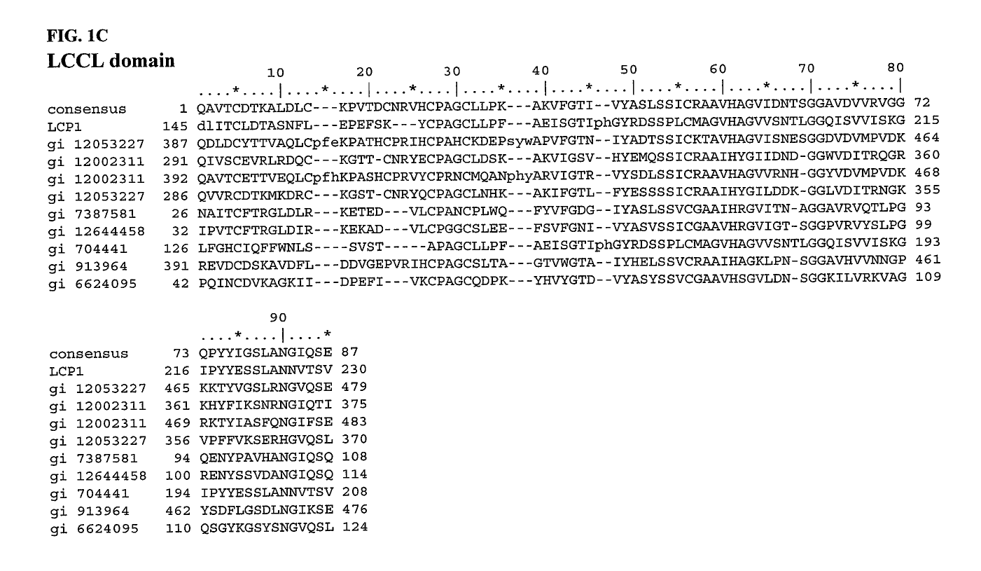

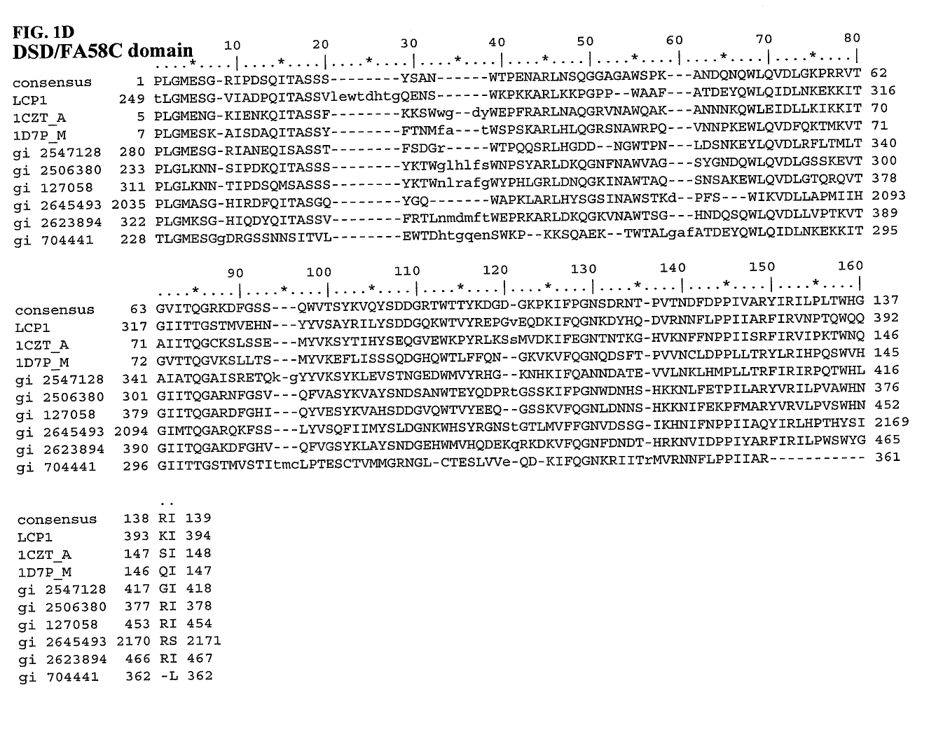

The newly isolated membrane protein LCP contains three distinct protein domains, including a CUB, an LCCL and a DSD/FA58C domain, respectively. The following four paragraphs describe the protein structure of LCP using LCP1 as an example. However, such description is also true for LCP2 except that, in comparison to LCP1, the LCP2 protein product lacks amino acid sequence 23-98 of LCP1, and therefore has a partial CUB domain the N-terminal of which is truncated. The structural features of LCP1 are schematized in FIG. 1.

LCP1 contains a CUB domain at residues 26-138 (http://smart.embl-heidelberg.de/) or alternatively at residues 26-141 (http://pfam.wustl.edu/). CUB is a protein domain with a predicted beta-barrel structure similar to that of immunoglobulins. It is an extracellular domain found in functionally diverse, mostly developmentally regulated proteins.

LCP1 has an LCCL domain at residues 147-230 (http://smart.embl-heidelberg.de/). First identified in Limulus factor C, Coch-5b2 and Lg11, the LCCL domain is hypothesized to have an antimicrobial function. Mutations in the LCCL domain have been shown to cause the deafness disorder DFNA9 in humans.

LCP1 also has a discoidin domain, also known as a F5/8 type C domain or an FA58C domain. In LCP1, the discoidin/FA58C domain occurs at residues 250-394 (discoidin domain, http://smart.embl-heidelberg.de/) or alternatively at residues 250-400 (F5/8 type C domain, http://pfam.wustl.edu/) or at residues 248-403 (FA58C, http://smart.embl-heidelberg.de/). The discoidin domain is a protein domain with a predicted amphipathic, membrane binding alpha helical structure at the C-terminal. This domain is found in a number of coagulation factors and has been shown to be responsible for phosphatidylserine-binding and essential for phosphatidylserine activity. The discoidin domain is also present in a subset of the tyrosine kinase receptor family known as discoidin domain receptors that are putatively involved in tumor progression.

The LCP1 protein contains a signal peptide consisting of the first 20 amino acid sequence of the protein. It also contains a transmembrane domain between amino acids 487 and 506 (http://www.ch.embnet.org/software/TMPRED_form.html). Other signatures of the newly isolated LCP1 proteins were identified by searching the PROSITE database (http://www.expasy.ch/tools/scnpsitl.html). These include six N-glycosylation sites (49-52, 109-112, 226-229, 428-431, 470-473, and 476-479), two cAMP- and cGMP-dependent protein kinase phosphorylation sites (313-316 and 512-515), six protein kinase C phosphorylation sites (130-132, 240-242, 279-281, 560-562, 592-594, and 654-656), thirteen casein kinase II phosphorylation sites, a single tyrosine kinase phosphorylation site (512-519), and twenty N-myristoylation sites.

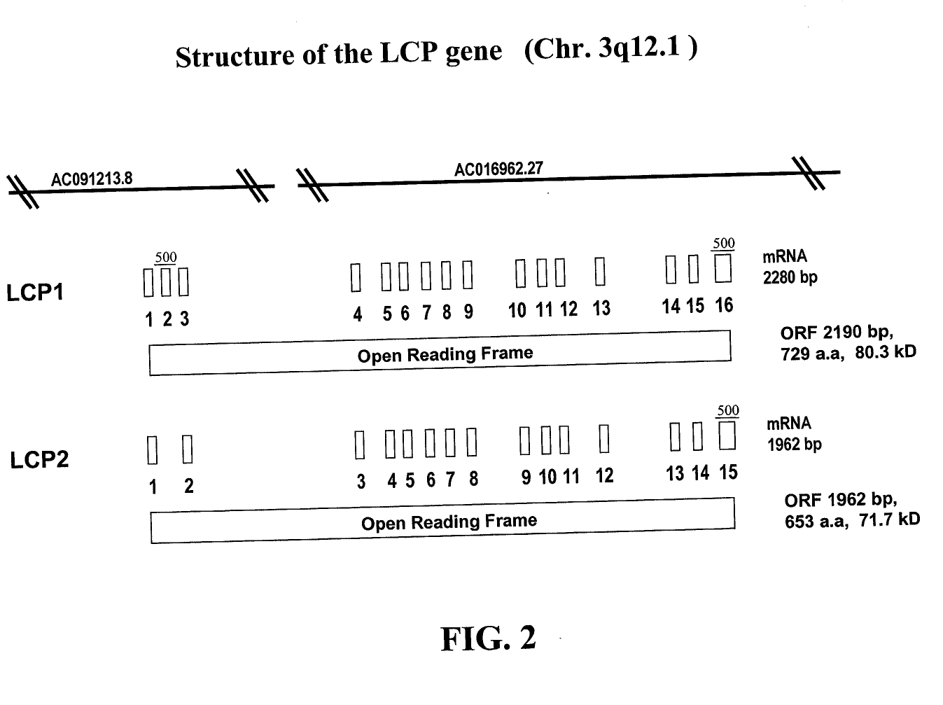

FIG. 2 shows the genomic organization of LCP.

At the top is shown the two bacterial artificial chromosomes (BACs), with GenBank accession numbers (AC091213.8, AC016962.27), which span the LCP locus. The genome-derived single-exon probes first used to demonstrate expression from this locus are shown above the exons and labeled “500”.

As shown in FIG. 2, LCP1 encodes a protein of 729 amino acids and is comprised of exons 1-16. LCP1 has a predicted molecular weight, prior to any post-translational modification, of 80.3 kD. LCP2 encodes a protein of 653 amino acids and is lacking exon 2 of LCP1. LCP2 has a predicted molecular weight, prior to any post-translational modification, of 80.3 kD.

As further discussed in the examples herein, expression of LCP was assessed using hybridization to genome-derived single exon microarrays and RT-PCR. Microarray analysis of exons two and sixteen of LCP1 showed expression in adrenal, adult liver, bone marrow, brain, fetal liver, heart, kidney, lung, placenta and prostate as well as a cell line, hela. RT-PCR confirmed microarray data, and further provided expression data for skeletal muscle and colon.

As more fully described below, the present invention provides isolated nucleic acids that encode LCP and fragments thereof. The invention further provides vectors for propagation and expression of the nucleic acids of the present invention, host cells comprising the nucleic acids and vectors of the present invention, proteins, protein fragments, and protein fusions of the present invention, and antibodies specific for all or any one of the isoforms. The invention provides pharmaceutical formulations of the nucleic acids, proteins, and antibodies of the present invention. The invention further provides transgenic cells and non-human organisms comprising human LCP nucleic acids, and transgenic cells and non-human organisms with targeted disruption of the endogenous orthologue of the human LCP. The invention additionally provides diagnostic, investigational, and therapeutic methods based on the LCP nucleic acids, proteins, and antibodies of the present invention.

Definitions

Unless defined otherwise, all technical and scientific terms used herein have the meaning commonly understood by one of ordinary skill in the art to which this invention belongs.

As used herein, “nucleic acid” (synonymously, “polynucleotide”) includes polynucleotides having natural nucleotides in native 5′-3′ phosphodiester linkage—e.g., DNA or RNA—as well as polynucleotides that have nonnatural nucleotide analogues, nonnative internucleoside bonds, or both, so long as the nonnatural polynucleotide is capable of sequence-discriminating basepairing under experimentally desired conditions. Unless otherwise specified, the term “nucleic acid” includes any topological conformation; the term thus explicitly comprehends single-stranded, double-stranded, partially duplexed, triplexed, hairpinned, circular, and padlocked conformations.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment.

For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature.

As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

As used herein, an isolated nucleic acid “encodes” a reference polypeptide when at least a portion of the nucleic acid, or its complement, can be directly translated to provide the amino acid sequence of the reference polypeptide, or when the isolated nucleic acid can be used, alone or as part of an expression vector, to express the reference polypeptide in vitro, in a prokaryotic host cell, or in a eukaryotic host cell.

As used herein, the term “exon” refers to a nucleic acid sequence found in genomic DNA that is bioinformatically predicted and/or experimentally confirmed to contribute contiguous sequence to a mature mRNA transcript.

As used herein, the phrase “open reading frame” and the equivalent acronym “ORF” refer to that portion of a transcript-derived nucleic acid that can be translated in its entirety into a sequence of contiguous amino acids. As so defined, an ORF has length, measured in nucleotides, exactly divisible by 3. As so defined, an ORF need not encode the entirety of a natural protein.

As used herein, the phrase “ORF-encoded peptide” refers to the predicted or actual translation of an ORF.

As used herein, the phrase “degenerate variant” of a reference nucleic acid sequence intends all nucleic acid sequences that can be directly translated, using the standard genetic code, to provide an amino acid sequence identical to that translated from the reference nucleic acid sequence.

As used herein, the term “microarray” and the equivalent phrase “nucleic acid microarray” refer to a substrate-bound collection of plural nucleic acids, hybridization to each of the plurality of bound nucleic acids being separately detectable. The substrate can be solid or porous, planar or non-planar, unitary or distributed.

As so defined, the term “microarray” and phrase “nucleic acid microarray” include all the devices so called in Schena (ed.), DNA Microarrays: A Practical Approach (Practical Approach Series), Oxford University Press (1999) (ISBN: 0199637768); Nature Genet. 21(1)(suppl):1-60 (1999); and Schena (ed.), Microarray Biochip: Tools and Technology, Eaton Publishing Company/BioTechniques Books Division (2000) (ISBN: 1881299376), the disclosures of which are incorporated herein by reference in their entireties.

As so defined, the term “microarray” and phrase “nucleic acid microarray” also include substrate-bound collections of plural nucleic acids in which the plurality of nucleic acids are distributably disposed on a plurality of beads, rather than on a unitary planar substrate, as is described, inter alia, in Brenner et al., Proc. Natl. Acad. Sci. USA 97(4):1665-1670 (2000), the disclosure of which is incorporated herein by reference in its entirety; in such case, the term “microarray” and phrase “nucleic acid microarray” refer to the plurality of beads in aggregate.

As used herein with respect to solution phase hybridization, the term “probe”, or equivalently, “nucleic acid probe” or “hybridization probe”, refers to an isolated nucleic acid of known sequence that is, or is intended to be, detectably labeled. As used herein with respect to a nucleic acid microarray, the term “probe” (or equivalently “nucleic acid probe” or “hybridization probe”) refers to the isolated nucleic acid that is, or is intended to be, bound to the substrate. In either such context, the term “target” refers to nucleic acid intended to be bound to probe by sequence complementarity.

As used herein, the expression “probe comprising SEQ ID NO:X”, and variants thereof, intends a nucleic acid probe, at least a portion of which probe has either (i) the sequence directly as given in the referenced SEQ ID NO:X, or (ii) a sequence complementary to the sequence as given in the referenced SEQ ID NO:X, the choice as between sequence directly as given and complement thereof dictated by the requirement that the probe be complementary to the desired target.

As used herein, the phrases “expression of a probe” and “expression of an isolated nucleic acid” and their linguistic equivalents intend that the probe or, (respectively, the isolated nucleic acid), or a probe (or, respectively, isolated nucleic acid) complementary in sequence thereto, can hybridize detectably under high stringency conditions to a sample of nucleic acids that derive from mRNA transcripts from a given source. For example, and by way of illustration only, expression of a probe in “liver” means that the probe can hybridize detectably under high stringency conditions to a sample of nucleic acids that derive from mRNA obtained from liver.

As used herein, “a single exon probe” comprises at least part of an exon (“reference exon”) and can hybridize detectably under high stringency conditions to transcript-derived nucleic acids that include the reference exon. The single exon probe will not, however, hybridize detectably under high stringency conditions to nucleic acids that lack the reference exon and that consist of one or more exons that are found adjacent to the reference exon in the genome.

For purposes herein, “high stringency conditions” are defined for solution phase hybridization as aqueous hybridization (i.e., free of formamide) in 6× SSC (where 20× SSC contains 3.0 M NaCl and 0.3 M sodium citrate), 1% SDS at 65° C. for at least 8 hours, followed by one or more washes in 0.2× SSC, 0.1% SDS at 65° C. “Moderate stringency conditions” are defined for solution phase hybridization as aqueous hybridization (i.e., free of formamide) in 6× SSC, 1% SDS at 65° C. for at least 8 hours, followed by one or more washes in 2× SSC, 0.1% SDS at room temperature.

For microarray-based hybridization, standard “high stringency conditions” are defined as hybridization in 50% formamide, 5× SSC, 0.2 μg/μl poly(dA), 0.2 μg/μl human cot1 DNA, and 0.5% SDS, in a humid oven at 42° C. overnight, followed by successive washes of the microarray in 1× SSC, 0.2% SDS at 55° C. for 5 minutes, and then 0.1× SSC, 0.2% SDS, at 55° C. for 20 minutes. For microarray-based hybridization, “moderate stringency conditions”, suitable for cross-hybridization to mRNA encoding structurally- and functionally-related proteins, are defined to be the same as those for high stringency conditions but with reduction in temperature for hybridization and washing to room temperature (approximately 25° C.).

As used herein, the terms “protein”, “polypeptide”, and “peptide” are used interchangeably to refer to a naturally-occurring or synthetic polymer of amino acid monomers (residues), irrespective of length, where amino acid monomer here includes naturally-occurring amino acids, naturally-occurring amino acid structural variants, and synthetic non-naturally occurring analogs that are capable of participating in peptide bonds. The terms “protein”, “polypeptide”, and “peptide” explicitly permits of post-translational and post-synthetic modifications, such as glycosylation.

The term “oligopeptide” herein denotes a protein, polypeptide, or peptide having 25 or fewer monomeric subunits.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment.

For example, a protein can be said to be “isolated” when it includes amino acid analogues or derivatives not found in nature, or includes linkages other than standard peptide bonds.

When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

A “purified protein” (equally, a purified polypeptide, peptide, or oligopeptide) is an isolated protein, as above described, present at a concentration of at least 95%, as measured on a weight basis with respect to total protein in a composition. A “substantially purified protein” (equally, a substantially purified polypeptide, peptide, or oligopeptide) is an isolated protein, as above described, present at a concentration of at least 70%, as measured on a weight basis with respect to total protein in a composition.

As used herein, the phrase “protein isoforms” refers to a plurality of proteins having nonidentical primary amino acid sequence but that share amino acid sequence encoded by at least one common exon.

As used herein, the phrase “alternative splicing” and its linguistic equivalents includes all types of RNA processing that lead to expression of plural protein isoforms from a single gene; accordingly, the phrase “splice variant(s)” and its linguistic equivalents embraces mRNAs transcribed from a given gene that, however processed, collectively encode plural protein isoforms. For example, and by way of illustration only, splice variants can include exon insertions, exon extensions, exon truncations, exon deletions, alternatives in the 5′ untranslated region (“5′ UT”) and alternatives in the 3′ untranslated region (“3′ UT”). Such 3′ alternatives include, for example, differences in the site of RNA transcript cleavage and site of poly(A) addition. See, e.g., Gautheret et al., Genome Res. 8:524-530 (1998).

As used herein, “orthologues” are separate occurrences of the same gene in multiple species. The separate occurrences have similar, albeit nonidentical, amino acid sequences, the degree of sequence similarity depending, in part, upon the evolutionary distance of the species from a common ancestor having the same gene.

As used herein, the term “paralogues” indicates separate occurrences of a gene in one species. The separate occurrences have similar, albeit nonidentical, amino acid sequences, the degree of sequence similarity depending, in part, upon the evolutionary distance from the gene duplication event giving rise to the separate occurrences.

As used herein, the term “homologues” is generic to “orthologues” and “paralogues”.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513), the disclosure of which is incorporated herein by reference in its entirety).

As used herein, antibodies can be produced by any known technique, including harvest from cell culture of native B lymphocytes, harvest from culture of hybridomas, recombinant expression systems, and phage display.

As used herein, “antigen” refers to a ligand that can be bound by an antibody; an antigen need not itself be immunogenic. The portions of the antigen that make contact with the antibody are denominated “epitopes”.

“Specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻⁹ M.

As used herein, “molecular binding partners”—and equivalently, “specific binding partners”—refer to pairs of molecules, typically pairs of biomolecules, that exhibit specific binding. Nonlimiting examples are receptor and ligand, antibody and antigen, and biotin to any of avidin, streptavidin, neutrAvidin and captAvidin.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “portion”, as used with respect to nucleic acids, proteins, and antibodies, is synonymous with “fragment”.

Nucleic Acid Molecules

In a first aspect, the invention provides isolated nucleic acids that encode LCP, variants having at least 65% sequence identity thereto, degenerate variants thereof, variants that encode LCP proteins having conservative or moderately conservative substitutions, cross-hybridizing nucleic acids, and fragments thereof.

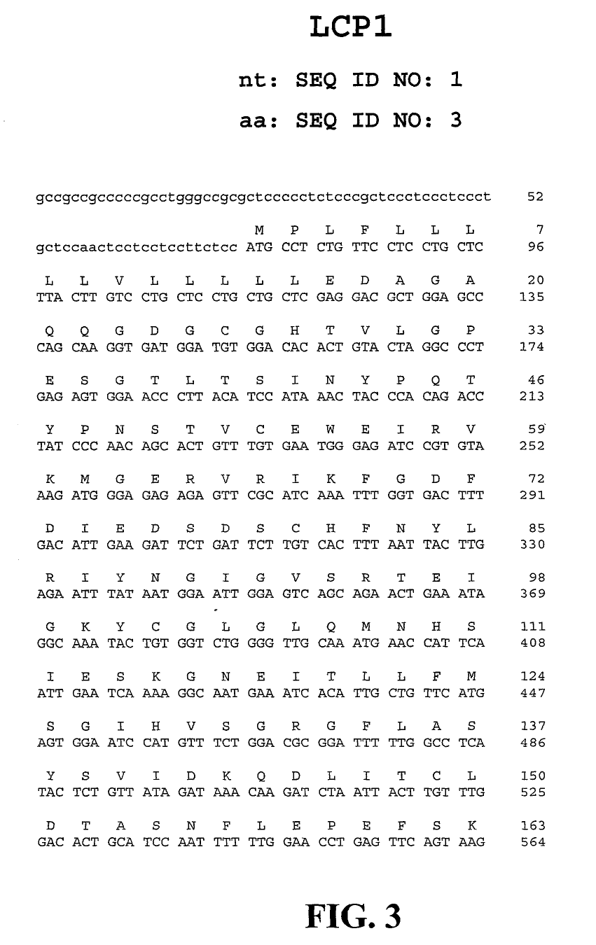

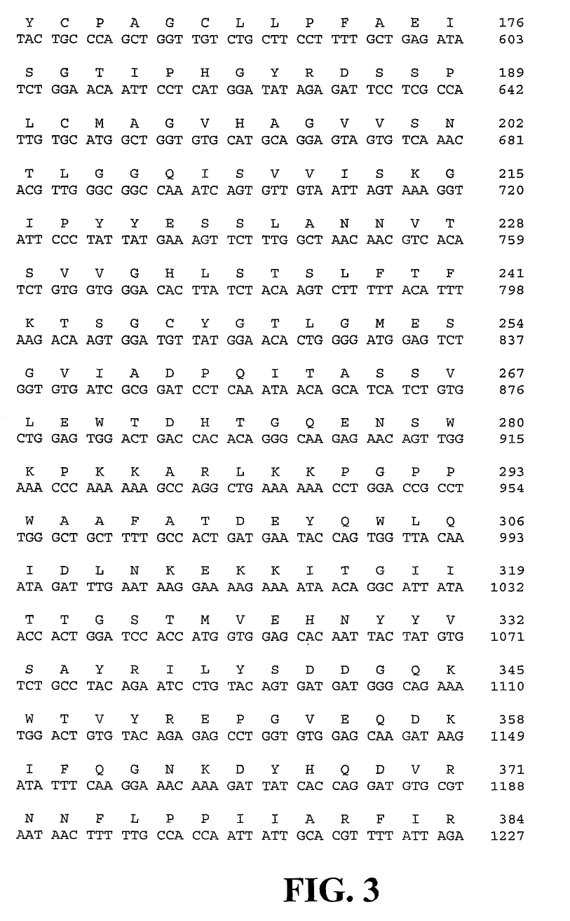

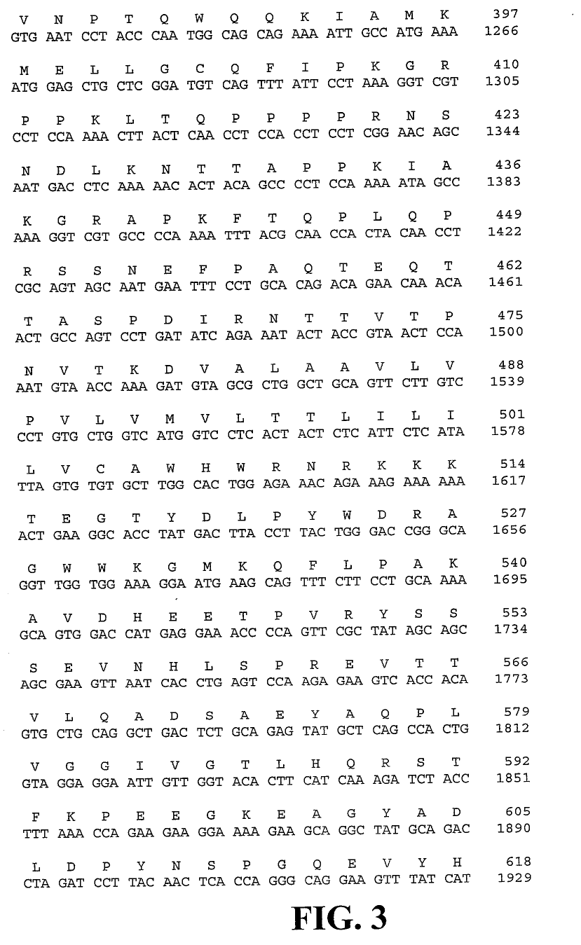

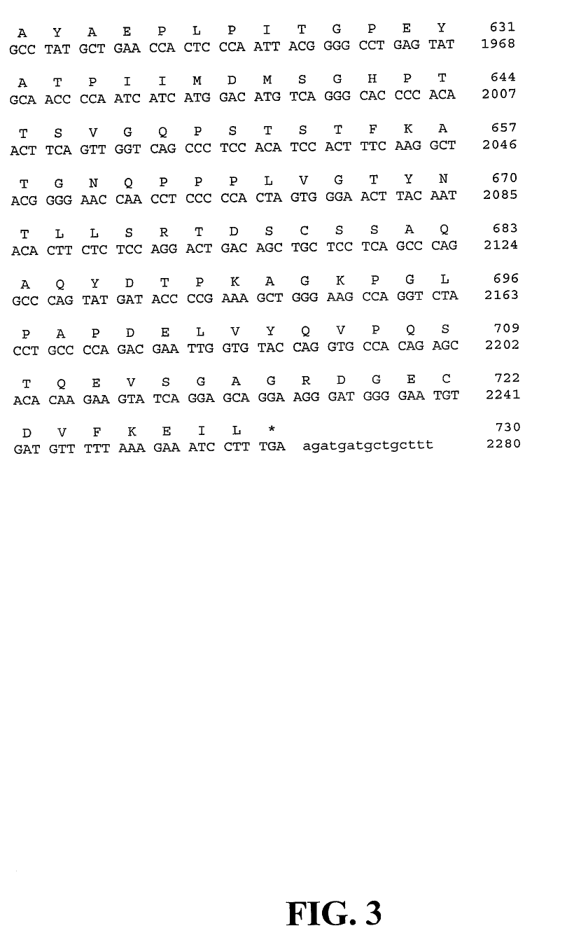

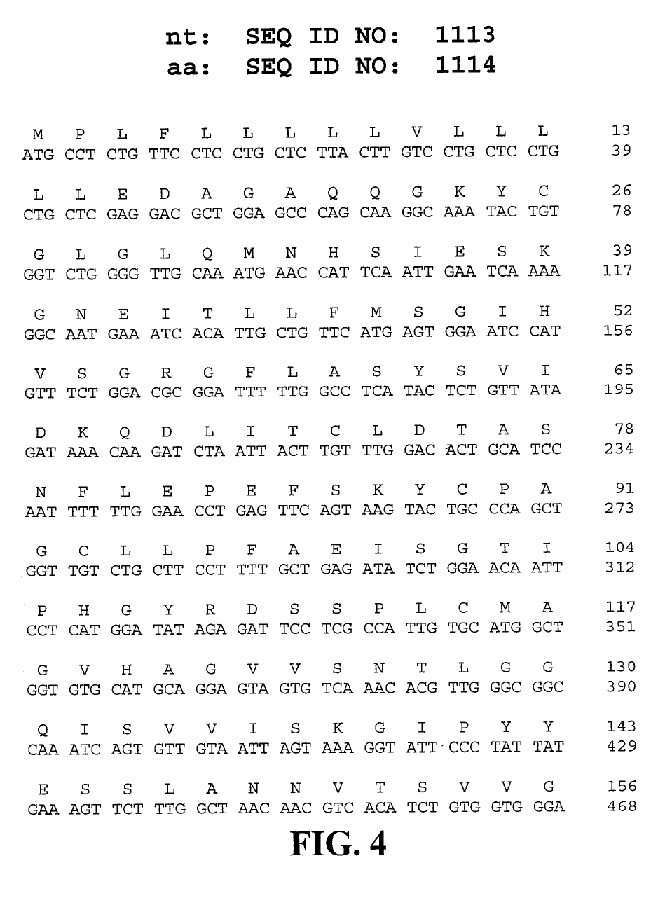

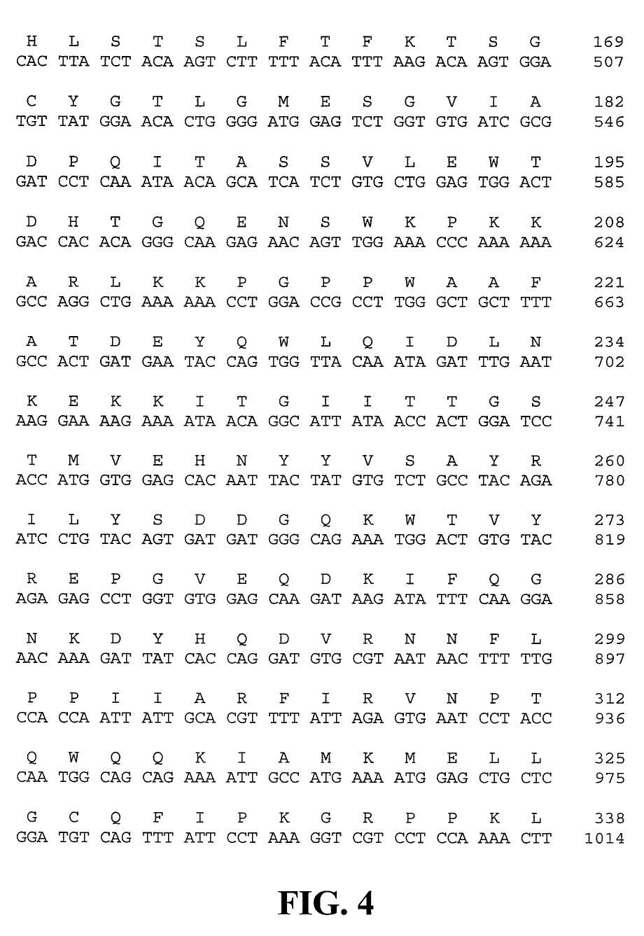

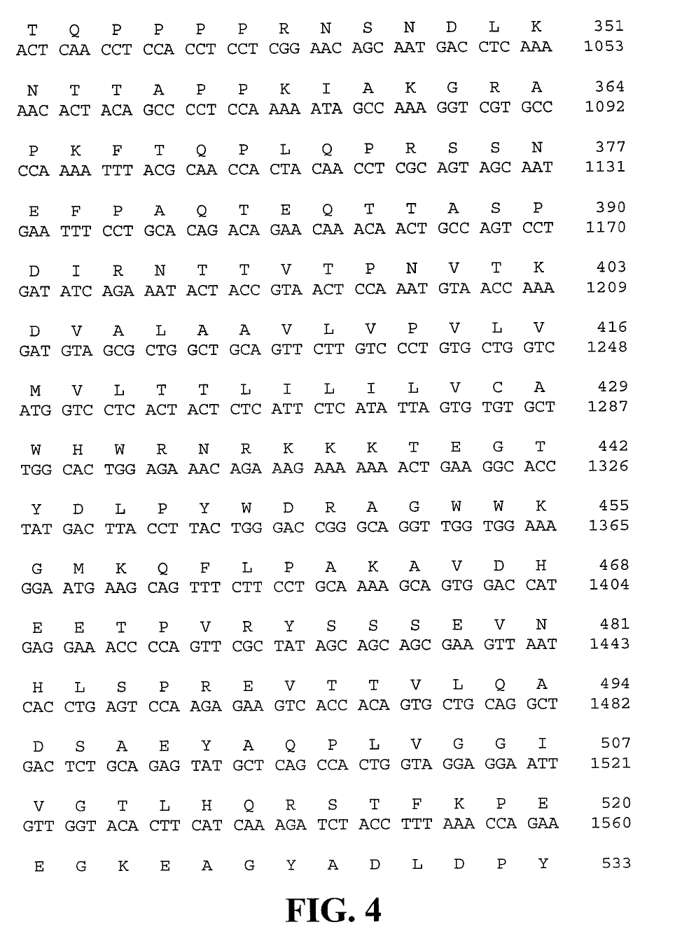

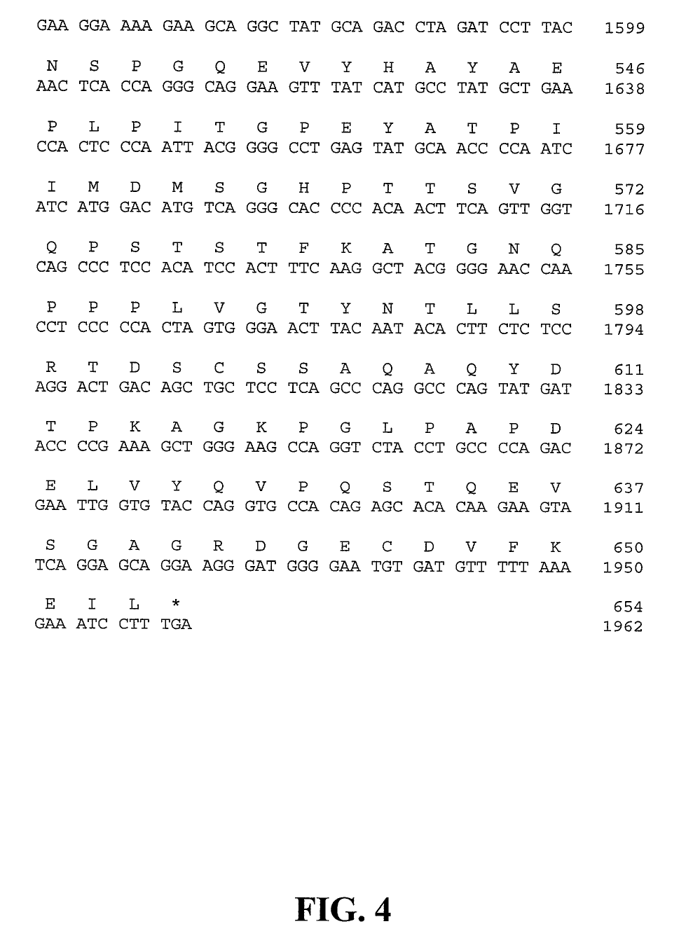

FIGS. 3 and 4 present the nucleotide sequence of the LCP cDNA clones, with predicted amino acid translation; the sequences are further presented in the Sequence Listing, incorporated herein by reference in its entirety, in SEQ ID NOs: 1 (full length nucleotide sequence of human LCP1 cDNA), 3 (full length amino acid coding sequence of human LCP1), 1113 (full length nucleotide sequence of human LCP2 cDNA) and 1114 (full length amino acid coding sequence of human LCP2).

Unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

Unless otherwise indicated, nucleotide sequences of the isolated nucleic acids of the present invention were determined by sequencing a DNA molecule that had resulted, directly or indirectly, from at least one enzymatic polymerization reaction (e.g., reverse transcription and/or polymerase chain reaction) using an automated sequencer (such as the MegaBACE™ 1000, Amersham Biosciences, Sunnyvale, Calif., USA), or by reliance upon such sequence or upon genomic sequence prior-accessioned into a public database. Unless otherwise indicated, all amino acid sequences of the polypeptides of the present invention were predicted by translation from the nucleic acid sequences so determined.

As a consequence, any nucleic acid sequence presented herein may contain errors introduced by erroneous incorporation of nucleotides during polymerization, by erroneous base calling by the automated sequencer (although such sequencing errors have been minimized for the nucleic acids directly determined herein, unless otherwise indicated, by the sequencing of each of the complementary strands of a duplex DNA), or by similar errors accessioned into the public database. Such errors can readily be identified and corrected by resequencing of the genomic locus using standard techniques.

Single nucleotide polymorphisms (SNPs) occur frequently in eukaryotic genomes—more than 1.4 million SNPs have already identified in the human genome, International Human Genome Sequencing Consortium, Nature 409:860-921 (2001)—and the sequence determined from one individual of a species may differ from other allelic forms present within the population. Additionally, small deletions and insertions, rather than single nucleotide polymorphisms, are not uncommon in the general population, and often do not alter the function of the protein.

Accordingly, it is an aspect of the present invention to provide nucleic acids not only identical in sequence to those described with particularity herein, but also to provide isolated nucleic acids at least about 65% identical in sequence to those described with particularity herein, typically at least about 70%, 75%, 80%, 85%, or 90% identical in sequence to those described with particularity herein, usefully at least about 91%, 92%, 93%, 94%, or 95% identical in sequence to those described with particularity herein, usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention, as by random or directed mutagenesis.

For purposes herein, percent identity of two nucleic acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program BLAST 2 SEQUENCES, available online at

http://www.ncbi.nlm.nih.gov/blast/bl2seq/bl2.html.

To assess percent identity of nucleic acids, the BLASTN module of BLAST 2 SEQUENCES is used with default values of (i) reward for a match: 1; (ii) penalty for a mismatch: −2; (iii) open gap 5 and extension gap 2 penalties; (iv) gap X_dropoff 50 expect 10 word size 11 filter, and both sequences are entered in their entireties.

As is well known, the genetic code is degenerate, with each amino acid except methionine translated from a plurality of codons, thus permitting a plurality of nucleic acids of disparate sequence to encode the identical protein. As is also well known, codon choice for optimal expression varies from species to species. The isolated nucleic acids of the present invention being useful for expression of LCP proteins and protein fragments, it is, therefore, another aspect of the present invention to provide isolated nucleic acids that encode LCP proteins and portions thereof not only identical in sequence to those described with particularity herein, but degenerate variants thereof as well.

As is also well known, amino acid substitutions occur frequently among natural allelic variants, with conservative substitutions often occasioning only de minimis change in protein function.

Accordingly, it is an aspect of the present invention to provide nucleic acids not only identical in sequence to those described with particularity herein, but also to provide isolated nucleic acids that encode LCP, and portions thereof, having conservative amino acid substitutions, and also to provide isolated nucleic acids that encode LCP, and portions thereof, having moderately conservative amino acid substitutions.

Although there are a variety of metrics for calling conservative amino acid substitutions, based primarily on either observed changes among evolutionarily related proteins or on predicted chemical similarity, for purposes herein a conservative replacement is any change having a positive value in the PAM250 log-likelihood matrix reproduced herein below (see Gonnet et al., Science 256(5062):1443-5 (1992)):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 2 | −1 | 0 | 0 | 0 | 0 | 0 | 0 | −1 | −1 | −1 | 0 | −1 | −2 | 0 | 1 | 1 | −4 | −2 | 0 |
| R | −1 | 5 | 0 | 0 | −2 | 2 | 0 | −1 | 1 | −2 | −2 | 3 | −2 | −3 | −1 | 0 | 0 | −2 | −2 | −2 |
| N | 0 | 0 | 4 | 2 | −2 | 1 | 1 | 0 | 1 | −3 | −3 | 1 | −2 | −3 | −1 | 1 | 0 | −4 | −1 | −2 |
| D | 0 | 0 | 2 | 5 | −3 | 1 | 3 | 0 | 0 | −4 | −4 | 0 | −3 | −4 | −1 | 0 | 0 | −5 | −3 | −3 |
| C | 0 | −2 | −2 | −3 | 12 | −2 | −3 | −2 | −1 | −1 | −2 | −3 | −1 | −1 | −3 | 0 | 0 | −1 | 0 | 0 |
| Q | 0 | 2 | 1 | 1 | −2 | 3 | 2 | −1 | 1 | −2 | −2 | 2 | −1 | −3 | 0 | 0 | 0 | −3 | −2 | −2 |
| E | 0 | 0 | 1 | 3 | −3 | 2 | 4 | −1 | 0 | −3 | −3 | 1 | −2 | −4 | 0 | 0 | 0 | −4 | −3 | −2 |
| G | 0 | −1 | 0 | 0 | −2 | −1 | −1 | 7 | −1 | −4 | −4 | −1 | −4 | −5 | −2 | 0 | −1 | −4 | −4 | −3 |
| H | −1 | 1 | 1 | 0 | −1 | 1 | 0 | −1 | 6 | −2 | −2 | 1 | −1 | 0 | −1 | 0 | 0 | −1 | 2 | −2 |
| I | −1 | −2 | −3 | −4 | −1 | −2 | −3 | −4 | −2 | 4 | 3 | −2 | 2 | 1 | −3 | −2 | −1 | −2 | −1 | 3 |
| L | −1 | −2 | −3 | −4 | −2 | −2 | −3 | −4 | −2 | 3 | 4 | −2 | 3 | 2 | −2 | −2 | −1 | −1 | 0 | 2 |
| K | 0 | 3 | 1 | 0 | −3 | 2 | 1 | −1 | 1 | −2 | −2 | 3 | −1 | −3 | −1 | 0 | 0 | −4 | −2 | −2 |
| M | −1 | −2 | −2 | −3 | −1 | −1 | −2 | −4 | −1 | 2 | 3 | −1 | 4 | 2 | −2 | −1 | −1 | −1 | 0 | 2 |
| F | −2 | −3 | −3 | −4 | −1 | −3 | −4 | −5 | 0 | 1 | 2 | −3 | 2 | 7 | −4 | −3 | −2 | 4 | 5 | 0 |
| P | 0 | −1 | −1 | −1 | −3 | 0 | 0 | −2 | −1 | −3 | −2 | −1 | −2 | −4 | 8 | 0 | 0 | −5 | −3 | −2 |
| S | 1 | 0 | 1 | 0 | 0 | 0 | 0 | 0 | 0 | −2 | −2 | 0 | −1 | −3 | 0 | 2 | 2 | −3 | −2 | −1 |
| T | 1 | 0 | 0 | 0 | 0 | 0 | 0 | −1 | 0 | −1 | −1 | 0 | −1 | −2 | 0 | 2 | 2 | −4 | −2 | 0 |
| W | −4 | −2 | −4 | −5 | −1 | −3 | −4 | −4 | −1 | −2 | −1 | −4 | −1 | 4 | −5 | −3 | −4 | 14 | 4 | −3 |
| Y | −2 | −2 | −1 | −3 | 0 | −2 | −3 | −4 | 2 | −1 | 0 | −2 | 0 | 5 | −3 | −2 | −2 | 4 | 8 | −1 |
| V | 0 | −2 | −2 | −3 | 0 | −2 | −2 | −3 | −2 | 3 | 2 | −2 | 2 | 0 | −2 | −1 | 0 | −3 | −1 | 3 |

For purposes herein, a “moderately conservative” replacement is any change having a nonnegative value in the PAM250 log-likelihood matrix reproduced herein above.

As is also well known in the art, relatedness of nucleic acids can also be characterized using a functional test, the ability of the two nucleic acids to base-pair to one another at defined hybridization stringencies.

It is, therefore, another aspect of the invention to provide isolated nucleic acids not only identical in sequence to those described with particularity herein, but also to provide isolated nucleic acids (“cross-hybridizing nucleic acids”) that hybridize under high stringency conditions (as defined herein below) to all or to a portion of various of the isolated LCP nucleic acids of the present invention (“reference nucleic acids”), as well as cross-hybridizing nucleic acids that hybridize under moderate stringency conditions to all or to a portion of various of the isolated LCP nucleic acids of the present invention.

Such cross-hybridizing nucleic acids are useful, inter alia, as probes for, and to drive expression of, proteins related to the proteins of the present invention as alternative isoforms, homologues, paralogues, and orthologues. Particularly useful orthologues are those from other primate species, such as chimpanzee, rhesus macaque, monkey, baboon, orangutan, and gorilla; from rodents, such as rats, mice, guinea pigs; from lagomorphs, such as rabbits; and from domestic livestock, such as cow, pig, sheep, horse, goat and chicken.

For purposes herein, high stringency conditions are defined as aqueous hybridization (i.e., free of formamide) in 6× SSC (where 20× SSC contains 3.0 M NaCl and 0.3 M sodium citrate), 1% SDS at 65° C. for at least 8 hours, followed by one or more washes in 0.2× SSC, 0.1% SDS at 65° C. For purposes herein, moderate stringency conditions are defined as aqueous hybridization (i.e., free of formamide) in 6× SSC, 1% SDS at 65° C. for at least 8 hours, followed by one or more washes in 2× SSC, 0.1% SDS at room temperature.

The hybridizing portion of the reference nucleic acid is typically at least 15 nucleotides in length, often at least 17 nucleotides in length. Often, however, the hybridizing portion of the reference nucleic acid is at least 20 nucleotides in length, 25 nucleotides in length, and even 30 nucleotides, 35 nucleotides, 40 nucleotides, and 50 nucleotides in length. Of course, cross-hybridizing nucleic acids that hybridize to a larger portion of the reference nucleic acid—for example, to a portion of at least 50 nt, at least 100 nt, at least 150 nt, 200 nt, 250 nt, 300 nt, 350 nt, 400 nt, 450 nt, or 500 nt or more—or even to the entire length of the reference nucleic acid, are also useful.

The hybridizing portion of the cross-hybridizing nucleic acid is at least 75% identical in sequence to at least a portion of the reference nucleic acid. Typically, the hybridizing portion of the cross-hybridizing nucleic acid is at least 80%, often at least 85%, 86%, 87%, 88%, 89% or even at least 90% identical in sequence to at least a portion of the reference nucleic acid. Often, the hybridizing portion of the cross-hybridizing nucleic acid will be at least 91%, 92%, 93%, 94%, 95%, 96%, 97%, 98%, or 99% identical in sequence to at least a portion of the reference nucleic acid sequence. At times, the hybridizing portion of the cross-hybridizing nucleic acid will be at least 99.5% identical in sequence to at least a portion of the reference nucleic acid.

The invention also provides fragments of various of the isolated nucleic acids of the present invention.

By “fragments” of a reference nucleic acid is here intended isolated nucleic acids, however obtained, that have a nucleotide sequence identical to a portion of the reference nucleic acid sequence, which portion is at least 17 nucleotides and less than the entirety of the reference nucleic acid. As so defined, “fragments” need not be obtained by physical fragmentation of the reference nucleic acid, although such provenance is not thereby precluded.

In theory, an oligonucleotide of 17 nucleotides is of sufficient length as to occur at random less frequently than once in the three gigabase human genome, and thus to provide a nucleic acid probe that can uniquely identify the reference sequence in a nucleic acid mixture of genomic complexity. As is well known, further specificity can be obtained by probing nucleic acid samples of subgenomic complexity, and/or by using plural fragments as short as 17 nucleotides in length collectively to prime amplification of nucleic acids, as, e.g., by polymerase chain reaction (PCR).

As further described herein below, nucleic acid fragments that encode at least 6 contiguous amino acids (i.e., fragments of 18 nucleotides or more) are useful in directing the expression or the synthesis of peptides that have utility in mapping the epitopes of the protein encoded by the reference nucleic acid. See, e.g., Geysen et al., “Use of peptide synthesis to probe viral antigens for epitopes to a resolution of a single amino acid,” Proc. Natl. Acad. Sci. USA 81:3998-4002 (1984); and U.S. Pat. Nos. 4,708,871 and 5,595,915, the disclosures of which are incorporated herein by reference in their entireties.

As further described herein below, fragments that encode at least 8 contiguous amino acids (i.e., fragments of 24 nucleotides or more) are useful in directing the expression or the synthesis of peptides that have utility as immunogens. See, e.g., Lerner, “Tapping the immunological repertoire to produce antibodies of predetermined specificity,” Nature 299:592-596 (1982); Shinnick et al., “Synthetic peptide immunogens as vaccines,” Annu. Rev. Microbiol. 37:425-46 (1983); Sutcliffe et al., “Antibodies that react with predetermined sites on proteins,” Science 219:660-6 (1983), the disclosures of which are incorporated herein by reference in their entireties.

The nucleic acid fragment of the present invention is thus at least 17 nucleotides in length, typically at least 18 nucleotides in length, and often at least 24 nucleotides in length. Often, the nucleic acid of the present invention is at least 25 nucleotides in length, and even 30 nucleotides, 35 nucleotides, 40 nucleotides, or 45 nucleotides in length. Of course, larger fragments having at least 50 nt, at least 100 nt, at least 150 nt, 200 nt, 250 nt, 300 nt, 350 nt, 400 nt, 450 nt, or 500 nt or more are also useful, and at times preferred.

Having been based upon the mining of genomic sequence, rather than upon surveillance of expressed message, the present invention further provides isolated genome-derived nucleic acids that include portions of the LCP gene.

The invention particularly provides genome-derived single exon probes.

As further described in commonly owned and copending U.S. patent application Ser. No. 09/864,761, filed May 23, 2001; Ser. No. 09/774,203, filed Jan. 29, 2001; and Ser. No. 09/632,366, filed Aug. 3, 2000, the disclosures of which are incorporated herein by reference in their entireties, “a single exon probe” comprises at least part of an exon (“reference exon”) and can hybridize detectably under high stringency conditions to transcript-derived nucleic acids that include the reference exon. The single exon probe will not, however, hybridize detectably under high stringency conditions to nucleic acids that lack the reference exon and instead consist of one or more exons that are found adjacent to the reference exon in the genome.

Genome-derived single exon probes typically further comprise, contiguous to a first end of the exon portion, a first intronic and/or intergenic sequence that is identically contiguous to the exon in the genome. Often, the genome-derived single exon probe further comprises, contiguous to a second end of the exonic portion, a second intronic and/or intergenic sequence that is identically contiguous to the exon in the genome.

The minimum length of genome-derived single exon probes is defined by the requirement that the exonic portion be of sufficient length to hybridize under high stringency conditions to transcript-derived nucleic acids. Accordingly, the exon portion is at least 17 nucleotides, typically at least 18 nucleotides, 20 nucleotides, 24 nucleotides, 25 nucleotides or even 30, 35, 40, 45, or 50 nucleotides in length, and can usefully include the entirety of the exon, up to 100 nt, 150 nt, 200 nt, 250 nt, 300 nt, 350 nt, 400 nt or even 500 nt or more in length.

The maximum length of genome-derived single exon probes is defined by the requirement that the probes contain portions of no more than one exon, that is, be unable to hybridize detectably under high stringency conditions to nucleic acids that lack the reference exon but include one or more exons that are found adjacent to the reference exon the genome.

Given variable spacing of exons through eukaryotic genomes, the maximum length of single exon probes of the present invention is typically no more than 25 kb, often no more than 20 kb, 15 kb, 10 kb or 7.5 kb, or even no more than 5 kb, 4 kb, 3 kb, or even no more than about 2.5 kb in length.

The genome-derived single exon probes of the present invention can usefully include at least a first terminal priming sequence not found in contiguity with the rest of the probe sequence in the genome, and often will contain a second terminal priming sequence not found in contiguity with the rest of the probe sequence in the genome.

The present invention also provides isolated genome-derived nucleic acids that include nucleic acid sequence elements that control transcription of the LCP gene.

With a complete draft of the human genome now available, genomic sequences that are within the vicinity of the LCP coding region (and that are additional to those described with particularity herein) can readily be obtained by PCR amplification.

The isolated nucleic acids of the present invention can be composed of natural nucleotides in native 5′-3′ phosphodiester internucleoside linkage—e.g., DNA or RNA—or can contain any or all of nonnatural nucleotide analogues, nonnative internucleoside bonds, or post-synthesis modifications, either throughout the length of the nucleic acid or localized to one or more portions thereof.

As is well known in the art, when the isolated nucleic acid is used as a hybridization probe, the range of such nonnatural analogues, nonnative internucleoside bonds, or post-synthesis modifications will be limited to those that permit sequence-discriminating basepairing of the resulting nucleic acid. When used to direct expression or RNA or protein in vitro or in vivo, the range of such nonnatural analogues, nonnative internucleoside bonds, or post-synthesis modifications will be limited to those that permit the nucleic acid to function properly as a polymerization substrate. When the isolated nucleic acid is used as a therapeutic agent, the range of such changes will be limited to those that do not confer toxicity upon the isolated nucleic acid.

For example, when desired to be used as probes, the isolated nucleic acids of the present invention can usefully include nucleotide analogues that incorporate labels that are directly detectable, such as radiolabels or fluorophores, or nucleotide analogues that incorporate labels that can be visualized in a subsequent reaction, such as biotin or various haptens.

Common radiolabeled analogues include those labeled with ³³P, ³²P, and ³⁵S, such as α-³²P-DATP, α-³²P-dCTP, α-³²P-dGTP, α-³²P-dTTP, α-³²P-3′dATP, α-³²P-ATP, α-³²P-CTP, α-³²P-GTP, α-32P-UTP, α-³⁵S-dATP, γ-³⁵S-GTP, γ-³³P-DATP, and the like.

Commercially available fluorescent nucleotide analogues readily incorporated into the nucleic acids of the present invention include Cy3-dCTP, Cy3-dUTP, Cy5-dCTP, Cy3-dUTP (Amersham Pharmacia Biotech, Piscataway, N.J., USA), fluorescein-12-dUTP, tetramethylrhodamine-6-dUTP, Texas Red®-5-dUTP, Cascade Blue®-7-dUTP, BODIPY® FL-14-dUTP, BODIPY® TMR-14-dUTP, BODIPY® TR-14-dUTP, Rhodamine Green™-5 dUTP, Oregon Green® 488-5-dUTP, Texas Red®-12-dUTP, BODIPY® 630/650-14-dUTP, BODIPY® 650/665-14-dUTP, Alexa Fluor® 488-5-dUTP, Alexa Fluor® 532-5-dUTP, Alexa Fluor® 568-5-dUTP, Alexa Fluor® 594-5-dUTP, Alexa Fluor® 546-14-dUTP, fluorescein-12-UTP, tetramethylrhodamine-6-UTP, Texas Red®-5-UTP, Cascade Blue®-7-UTP, BODIPY® FL-14-UTP, BODIPY® TMR-14-UTP, BODIPY® TR-14-UTP, Rhodamine Green™-5-UTP, Alexa Fluor® 488-5-UTP, Alexa Fluor® 546-14-UTP (Molecular Probes, Inc. Eugene, Oreg., USA).

Protocols are available for custom synthesis of nucleotides having other fluorophores. Henegariu et al., “Custom Fluorescent-Nucleotide Synthesis as an Alternative Method for Nucleic Acid Labeling,” Nature Biotechnol. 18:345-348 (2000), the disclosure of which is incorporated herein by reference in its entirety.

Haptens that are commonly conjugated to nucleotides for subsequent labeling include biotin (biotin-11-dUTP, Molecular Probes, Inc., Eugene, Oreg., USA; biotin-21-UTP, biotin-21-dUTP, Clontech Laboratories, Inc., Palo Alto, Calif., USA), digoxigenin (DIG-11-dUTP, alkali labile, DIG-11-UTP, Roche Diagnostics Corp., Indianapolis, Ind., USA), and dinitrophenyl (dinitrophenyl-11-dUTP, Molecular Probes, Inc., Eugene, Oreg., USA).

As another example, when desired to be used for antisense inhibition of transcription or translation, the isolated nucleic acids of the present invention can usefully include altered, often nuclease-resistant, internucleoside bonds. See Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents—Symposium No. 209, John Wiley & Son Ltd (1997) (ISBN: 0471972797), the disclosures of which are incorporated herein by reference in their entireties. Such altered internucloside bonds are often desired also when the isolated nucleic acid of the present invention is to be used for targeted gene correction, Gamper et al., Nucl. Acids Res. 28(21):4332-4339 (2000), the disclosures of which are incorporated herein by reference in its entirety.

Modified oligonucleotide backbones often preferred when the nucleic acid is to be used for antisense purposes are, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosures of which are incorporated herein by reference in their entireties.

Preferred modified oligonucleotide backbones for antisense use that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above backbones include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, the disclosures of which are incorporated herein by reference in their entireties.

In other preferred oligonucleotide mimetics, both the sugar and the internucleoside linkage are replaced with novel groups, such as peptide nucleic acids (PNA).

In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza nitrogen atoms of the amide portion of the backbone, typically by methylene carbonyl linkages.

The uncharged nature of the PNA backbone provides PNA/DNA and PNA/RNA duplexes with a higher thermal stability than is found in DNA/DNA and DNA/RNA duplexes, resulting from the lack of charge repulsion between the PNA and DNA or RNA strand. In general, the Tm of a PNA/DNA or PNA/RNA duplex is 1° C. higher per base pair than the Tm of the corresponding DNA/DNA or DNA/RNA duplex (in 100 mM NaCl).

The neutral backbone also allows PNA to form stable DNA duplexes largely independent of salt concentration. At low ionic strength, PNA can be hybridized to a target sequence at temperatures that make DNA hybridization problematic or impossible. And unlike DNA/DNA duplex formation, PNA hybridization is possible in the absence of magnesium. Adjusting the ionic strength, therefore, is useful if competing DNA or RNA is present in the sample, or if the nucleic acid being probed contains a high level of secondary structure.

PNA also demonstrates greater specificity in binding to complementary DNA. A PNA/DNA mismatch is more destabilizing than DNA/DNA mismatch. A single mismatch in mixed a PNA/DNA 15-mer lowers the Tm by 8-20° C. (15° C. on average). In the corresponding DNA/DNA duplexes, a single mismatch lowers the Tm by 4-16° C. (11° C. on average). Because PNA probes can be significantly shorter than DNA probes, their specificity is greater.

Additionally, nucleases and proteases do not recognize the PNA polyamide backbone with nucleobase sidechains. As a result, PNA oligomers are resistant to degradation by enzymes, and the lifetime of these compounds is extended both in vivo and in vitro. In addition, PNA is stable over a wide pH range.

Because its backbone is formed from amide bonds, PNA can be synthesized using a modified peptide synthesis protocol. PNA oligomers can be synthesized by both Fmoc and tBoc methods. Representative U.S. patents that teach the preparation of PNA compounds include, but are not limited to, U.S. Pat. Nos. 5,539,082; 5,714,331; and 5,719,262, each of which is herein incorporated by reference; automated PNA synthesis is readily achievable on commercial synthesizers (see, e.g., “PNA User's Guide,” Rev. 2, February 1998, Perseptive Biosystems Part No. 60138, Applied Biosystems, Inc., Foster City, Calif.).

PNA chemistry and applications are reviewed, inter alia, in Ray et al., FASEB J. 14(9):1041-60 (2000); Nielsen et al., Pharmacol Toxicol. 86(1):3-7 (2000); Larsen et al., Biochim Biophys Acta. 1489(1):159-66 (1999); Nielsen, Curr. Opin. Struct. Biol. 9(3):353-7 (1999), and Nielsen, Curr. Opin. Biotechnol. 10(1):71-5 (1999), the disclosures of which are incorporated herein by reference in their entireties.

Differences from nucleic acid compositions found in nature—e.g., nonnative bases, altered internucleoside linkages, post-synthesis modification—can be present throughout the length of the nucleic acid or can, instead, usefully be localized to discrete portions thereof. As an example of the latter, chimeric nucleic acids can be synthesized that have discrete DNA and RNA domains and demonstrated utility for targeted gene repair, as further described in U.S. Pat. Nos. 5,760,012 and 5,731,181, the disclosures of which are incorporated herein by reference in their entireties. As another example, chimeric nucleic acids comprising both DNA and PNA have been demonstrated to have utility in modified PCR reactions. See Misra et al., Biochem. 37: 1917-1925 (1998); see also Finn et al., Nucl. Acids Res. 24: 3357-3363 (1996), incorporated herein by reference.

Unless otherwise specified, nucleic acids of the present invention can include any topological conformation appropriate to the desired use; the term thus explicitly comprehends, among others, single-stranded, double-stranded, triplexed, quadruplexed, partially double-stranded, partially-triplexed, partially-quadruplexed, branched, hairpinned, circular, and padlocked conformations. Padlock conformations and their utilities are further described in Baner et al., Curr. Opin. Biotechnol. 12:11-15 (2001); Escude et al., Proc. Natl. Acad. Sci. USA 14;96(19):10603-7 (1999); Nilsson et al., Science 265(5181):2085-8 (1994), the disclosures of which are incorporated herein by reference in their entireties. Triplex and quadruplex conformations, and their utilities, are reviewed in Praseuth et al., Biochim. Biophys. Acta. 1489(1):181-206 (1999); Fox, Curr. Med. Chem. 7(1):17-37 (2000); Kochetkova et al., Methods Mol. Biol. 130:189-201 (2000); Chan et al., J. Mol. Med. 75(4):267-82 (1997), the disclosures of which are incorporated herein by reference in their entireties.

The nucleic acids of the present invention can be detectably labeled.

Commonly-used labels include radionuclides, such as ³²P, ³³P, ³⁵S, ³H (and for NMR detection, ¹³C and ¹⁵N), haptens that can be detected by specific antibody or high affinity binding partner (such as avidin), and fluorophores.

As noted above, detectable labels can be incorporated by inclusion of labeled nucleotide analogues in the nucleic acid. Such analogues can be incorporated by enzymatic polymerization, such as by nick translation, random priming, polymerase chain reaction (PCR), terminal transferase tailing, and end-filling of overhangs, for DNA molecules, and in vitro transcription driven, e.g., from phage promoters, such as T7, T3, and SP6, for RNA molecules. Commercial kits are readily available for each such labeling approach.

Analogues can also be incorporated during automated solid phase chemical synthesis.

As is well known, labels can also be incorporated after nucleic acid synthesis, with the 5′ phosphate and 3′ hydroxyl providing convenient sites for post-synthetic covalent attachment of detectable labels.

Various other post-synthetic approaches permit internal labeling of nucleic acids.

For example, fluorophores can be attached using a cisplatin reagent that reacts with the N7 of guanine residues (and, to a lesser extent, adenine bases) in DNA, RNA, and PNA to provide a stable coordination complex between the nucleic acid and fluorophore label (Universal Linkage System) (available from Molecular Probes, Inc., Eugene, Oreg., USA and Amersham Pharmacia Biotech, Piscataway, N.J., USA); see Alers et al., Genes, Chromosomes & Cancer, Vol. 25, pp. 301-305 (1999); Jelsma et al., J. NIH Res. 5:82 (1994); Van Belkum et al., BioTechniques 16:148-153 (1994), incorporated herein by reference. As another example, nucleic acids can be labeled using a disulfide-containing linker (FastTag™ Reagent, Vector Laboratories, Inc., Burlingame, Calif., USA) that is photo- or thermally coupled to the target nucleic acid using aryl azide chemistry; after reduction, a free thiol is available for coupling to a hapten, fluorophore, sugar, affinity ligand, or other marker.

Multiple independent or interacting labels can be incorporated into the nucleic acids of the present invention.

For example, both a fluorophore and a moiety that in proximity thereto acts to quench fluorescence can be included to report specific hybridization through release of fluorescence quenching, Tyagi et al., Nature Biotechnol. 14: 303-308 (1996); Tyagi et al., Nature Biotechnol. 16, 49-53 (1998); Sokol et al., Proc. Natl. Acad. Sci. USA 95: 11538-11543 (1998); Kostrikis et al., Science 279:1228-1229 (1998); Marras et al., Genet. Anal. 14: 151-156 (1999); U.S. Pat. Nos. 5,846,726, 5,925,517, 5925517, or to report exonucleotidic excision, U.S. Pat. No. 5,538,848; Holland et al., Proc. Natl. Acad. Sci: USA 88:7276-7280 (1991); Heid et al., Genome Res. 6(10):986-94 (1996); Kuimelis et al., Nucleic Acids Symp Ser. (37):255-6 (1997); U.S. Pat. No. 5,723,591, the disclosures of which are incorporated herein by reference in their entireties.

So labeled, the isolated nucleic acids of the present invention can be used as probes, as further described below.

Nucleic acids of the present invention can also usefully be bound to a substrate. The substrate can porous or solid, planar or non-planar, unitary or distributed; the bond can be covalent or noncovalent. Bound to a substrate, nucleic acids of the present invention can be used as probes in their unlabeled state.

For example, the nucleic acids of the present invention can usefully be bound to a porous substrate, commonly a membrane, typically comprising nitrocellulose, nylon, or positively-charged derivatized nylon; so attached, the nucleic acids of the present invention can be used to detect LCP nucleic acids present within a labeled nucleic acid sample, either a sample of genomic nucleic acids or a sample of transcript-derived nucleic acids, e.g. by reverse dot blot.

The nucleic acids of the present invention can also usefully be bound to a solid substrate, such as glass, although other solid materials, such as amorphous silicon, crystalline silicon, or plastics, can also be used. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof.

Typically, the solid substrate will be rectangular, although other shapes, particularly disks and even spheres, present certain advantages. Particularly advantageous alternatives to glass slides as support substrates for array of nucleic acids are optical discs, as described in Demers, “Spatially Addressable Combinatorial Chemical Arrays in CD-ROM Format,” international patent publication WO 98/12559, incorporated herein by reference in its entirety.

The nucleic acids of the present invention can be attached covalently to a surface of the support substrate or applied to a derivatized surface in a chaotropic agent that facilitates denaturation and adherence by presumed noncovalent interactions, or some combination thereof.

The nucleic acids of the present invention can be bound to a substrate to which a plurality of other nucleic acids are concurrently bound, hybridization to each of the plurality of bound nucleic acids being separately detectable. At low density, e.g. on a porous membrane, these substrate-bound collections are typically denominated macroarrays; at higher density, typically on a solid support, such as glass, these substrate bound collections of plural nucleic acids are colloquially termed microarrays. As used herein, the term microarray includes arrays of all densities. It is, therefore, another aspect of the invention to provide microarrays that include the nucleic acids of the present invention.

The isolated nucleic acids of the present invention can be used as hybridization probes to detect, characterize, and quantify LCP nucleic acids in, and isolate LCP nucleic acids from, both genomic and transcript-derived nucleic acid samples. When free in solution, such probes are typically, but not invariably, detectably labeled; bound to a substrate, as in a microarray, such probes are typically, but not invariably unlabeled.

For example, the isolated nucleic acids of the present invention can be used as probes to detect and characterize gross alterations in the LCP genomic locus, such as deletions, insertions, translocations, and duplications of the LCP genomic locus through fluorescence in situ hybridization (FISH) to chromosome spreads. See, e.g., Andreeff et al. (eds.), Introduction to Fluorescence In Situ Hybridization: Principles and Clinical Applications, John Wiley & Sons (1999) (ISBN: 0471013455), the disclosure of which is incorporated herein by reference in its entirety. The isolated nucleic acids of the present invention can be used as probes to assess smaller genomic alterations using, e.g., Southern blot detection of restriction fragment length polymorphisms. The isolated nucleic acids of the present invention can be used as probes to isolate genomic clones that include the nucleic acids of the present invention, which thereafter can be restriction mapped and sequenced to identify deletions, insertions, translocations, and substitutions (single nucleotide polymorphisms, SNPs) at the sequence level.

The isolated nucleic acids of the present invention can also be used as probes to detect, characterize, and quantify LCP nucleic acids in, and isolate LCP nucleic acids from, transcript-derived nucleic acid samples.

For example, the isolated nucleic acids of the present invention can be used as hybridization probes to detect, characterize by length, and quantify LCP mRNA by northern blot of total or poly-A⁺-selected RNA samples. For example, the isolated nucleic acids of the present invention can be used as hybridization probes to detect, characterize by location, and quantify LCP message by in situ hybridization to tissue sections (see, e.g., Schwarchzacher et al., In Situ Hybridization, Springer-Verlag New York (2000) (ISBN: 0387915966), the disclosure of which is incorporated herein by reference in its entirety). For example, the isolated nucleic acids of the present invention can be used as hybridization probes to measure the representation of LCP clones in a cDNA library. For example, the isolated nucleic acids of the present invention can be used as hybridization probes to isolate LCP nucleic acids from cDNA libraries, permitting sequence level characterization of LCP messages, including identification of deletions, insertions, truncations—including deletions, insertions, and truncations of exons in alternatively spliced forms—and single nucleotide polymorphisms.

All of the aforementioned probe techniques are well within the skill in the art, and are described at greater length in standard texts such as Sambrook et al., Molecular Cloning: A Laboratory Manual (3^{rd} ed.), Cold Spring Harbor Laboratory Press (2001) (ISBN: 0879695773); Ausubel et al. (eds.), Short Protocols in Molecular Biology: A Compendium of Methods from Current Protocols in Molecular Biology (4^{th} ed.), John Wiley & Sons, 1999 (ISBN: 047132938X); and Walker et al. (eds.), The Nucleic Acids Protocols Handbook, Humana Press (2000) (ISBN: 0896034593), the disclosures of which are incorporated herein by reference in their entirety.

As described in the Examples herein below, the nucleic acids of the present invention can also be used to detect and quantify LCP nucleic acids in transcript-derived samples—that is, to measure expression of the LCP gene—when included in a microarray. Measurement of LCP expression has particular utility as LCP proteins have potential therapeutic as well as diagnostic roles for neurological and developmental disorders, as well as diseases involving cell-cell adhesion process, as further described in the Examples herein below.

As would be readily apparent to one of skill in the art, each LCP nucleic acid probe—whether labeled, substrate-bound, or both—is thus currently available for use as a tool for measuring the level of LCP expression in each of the tissues in which expression has already been confirmed, notably adrenal, adult liver, bone marrow, brain, fetal liver, heart, kidney, lung, placenta, skeletal muscle, colon and prostate. The utility is specific to the probe: under high stringency conditions, the probe reports the level of expression of message specifically containing that portion of the LCP gene included within the probe.

Measuring tools are well known in many arts, not just in molecular biology, and are known to possess credible, specific, and substantial utility. For example, U.S. Pat. No. 6,016,191 describes and claims a tool for measuring characteristics of fluid flow in a hydrocarbon well; U.S. Pat. No. 6,042,549 describes and claims a device for measuring exercise intensity; U.S. Pat. No. 5,889,351 describes and claims a device for measuring viscosity and for measuring characteristics of a fluid; U.S. Pat. No. 5,570,694 describes and claims a device for measuring blood pressure; U.S. Pat. No. 5,930,143 describes and claims a device for measuring the dimensions of machine tools; U.S. Pat. No. 5,279,044 describes and claims a measuring device for determining an absolute position of a movable element; U.S. Pat. No. 5,186,042 describes and claims a device for measuring action force of a wheel; and U.S. Pat. No. 4,246,774 describes and claims a device for measuring the draft of smoking articles such as cigarettes.

As for tissues not yet demonstrated to express LCP, the LCP nucleic acid probes of the present invention are currently available as tools for surveying such tissues to detect the presence of LCP nucleic acids.

Survey tools—i.e., tools for determining the presence and/or location of a desired object by search of an area—are well known in many arts, not just in molecular biology, and are known to possess credible, specific, and substantial utility. For example, U.S. Pat. No. 6,046,800 describes and claims a device for surveying an area for objects that move; U.S. Pat. No. 6,025,201 describes and claims an apparatus for locating and discriminating platelets from non-platelet particles or cells on a cell-by-cell basis in a whole blood sample; U.S. Pat. No. 5,990,689 describes and claims a device for detecting and locating anomalies in the electromagnetic protection of a system; U.S. Pat. No. 5,984,175 describes and claims a device for detecting and identifying wearable user identification units; U.S. Pat. No. 3,980,986 (“Oil well survey tool”), describes and claims a tool for finding the position of a drill bit working at the bottom of a borehole.

As noted above, the nucleic acid probes of the present invention are useful in constructing microarrays; the microarrays, in turn, are products of manufacture that are useful for measuring and for surveying gene expression.

When included on a microarray, each LCP nucleic acid probe makes the microarray specifically useful for detecting that portion of the LCP gene included within the probe, thus imparting upon the microarray device the ability to detect a signal where, absent such probe, it would have reported no signal. This utility makes each individual probe on such microarray akin to an antenna, circuit, firmware or software element included in an electronic apparatus, where the antenna, circuit, firmware or software element imparts upon the apparatus the ability newly and additionally to detect signal in a portion of the radio-frequency spectrum where previously it could not; such devices are known to have specific, substantial, and credible utility.

Changes in the level of expression need not be observed for the measurement of expression to have utility.

For example, where gene expression analysis is used to assess toxicity of chemical agents on cells, the failure of the agent to change a gene's expression level is evidence that the drug likely does not affect the pathway of which the gene's expressed protein is a part. Analogously, where gene expression analysis is used to assess side effects of pharmacologic agents—whether in lead compound discovery or in subsequent screening of lead compound derivatives—the inability of the agent to alter a gene's expression level is evidence that the drug does not affect the pathway of which the gene's expressed protein is a part.

WO 99/58720, incorporated herein by reference in its entirety, provides methods for quantifying the relatedness of a first and second gene expression profile and for ordering the relatedness of a plurality of gene expression profiles, without regard to the identity or function of the genes whose expression is used in the calculation.

Gene expression analysis, including gene expression analysis by microarray hybridization, is, of course, principally a laboratory-based art. Devices and apparatus used principally in laboratories to facilitate laboratory research are well-established to possess specific, substantial, and credible utility. For example, U.S. Pat. No. 6,001,233 describes and claims a gel electrophoresis apparatus having a cam-activated clamp; for example, U.S. Pat. No. 6,051,831 describes and claims a high mass detector for use in time-of-flight mass spectrometers; for example, U.S. Patent No. 5,824,269 describes and claims a flow cytometer-as is well known, few gel electrophoresis apparatuses, TOF-MS devices, or flow cytometers are sold for consumer use.

Indeed, and in particular, nucleic acid microarrays, as devices intended for laboratory use in measuring gene expression, are well-established to have specific, substantial and credible utility. Thus, the microarrays of the present invention have at least the specific, substantial and credible utilities of the microarrays claimed as devices and articles of manufacture in the following U.S. Patents, the disclosures of each of which is incorporated herein by reference: U.S. Pat. No. 5,445,934 (“Array of oligonucleotides on a solid substrate”); U.S. Pat. No. 5,744,305 (“Arrays of materials attached to a substrate”); and U.S. Pat. No. 6,004,752 (“Solid support with attached molecules”).

Genome-derived single exon probes and genome-derived single exon probe microarrays have the additional utility, inter alia, of permitting high-throughput detection of splice variants of the nucleic acids of the present invention, as further described in copending and commonly owned U.S. patent application Ser. No. 09/632,366, filed Aug. 3, 2000, the disclosure of which is incorporated herein by reference in its entirety.

The isolated nucleic acids of the present invention can also be used to prime synthesis of nucleic acid, for purpose of either analysis or isolation, using mRNA, cDNA, or genomic DNA as template.

For use as primers, at least 17 contiguous nucleotides of the isolated nucleic acids of the present invention will be used. Often, at least 18, 19, or 20 contiguous nucleotides of the nucleic acids of the present invention will be used, and on occasion at least 20, 22, 24, or 25 contiguous nucleotides of the nucleic acids of the present invention will be used, and even 30 nucleotides or more of the nucleic acids of the present invention can be used to prime specific synthesis.

The nucleic acid primers of the present invention can be used, for example, to prime first strand cDNA synthesis on an mRNA template.

Such primer extension can be done directly to analyze the message. Alternatively, synthesis on an mRNA template can be done to produce first strand cDNA. The first strand cDNA can thereafter be used, inter alia, directly as a single-stranded probe, as above-described, as a template for sequencing—permitting identification of alterations, including deletions, insertions, and substitutions, both normal allelic variants and mutations associated with abnormal phenotypes—or as a template, either for second strand cDNA synthesis (e.g., as an antecedent to insertion into a cloning or expression vector), or for amplification.

The nucleic acid primers of the present invention can also be used, for example, to prime single base extension (SBE) for SNP detection (see, e.g., U.S. Pat. No. 6,004,744, the disclosure of which is incorporated herein by reference in its entirety).

As another example, the nucleic acid primers of the present invention can be used to prime amplification of LCP nucleic acids, using transcript-derived or genomic DNA as template.

Primer-directed amplification methods are now well-established in the art. Methods for performing the polymerase chain reaction (PCR) are compiled, inter alia, in McPherson, PCR (Basics: From Background to Bench), Springer Verlag (2000) (ISBN: 0387916008); Innis et al. (eds.), PCR Applications: Protocols for Functional Genomics, Academic Press (1999) (ISBN: 0123721857); Gelfand et al. (eds.), PCR Strategies, Academic Press (1998) (ISBN: 0123721822); Newton et al., PCR, Springer-Verlag New York (1997) (ISBN: 0387915060); Burke (ed.), PCR: Essential Techniques, John Wiley & Son Ltd (1996) (ISBN: 047195697X); White (ed.), PCR Cloning Protocols: From Molecular Cloning to Genetic Engineering, Vol. 67, Humana Press (1996) (ISBN: 0896033430); McPherson et al. (eds.), PCR 2: A Practical Approach, Oxford University Press, Inc. (1995) (ISBN: 0199634254), the disclosures of which are incorporated herein by reference in their entireties. Methods for performing RT-PCR are collected, e.g., in Siebert et al. (eds.), Gene Cloning and Analysis by RT-PCR, Eaton Publishing Company/Bio Techniques Books Division, 1998 (ISBN: 1881299147); Siebert (ed.), PCR Technique:RT-PCR, Eaton Publishing Company/BioTechniques Books (1995) (ISBN:1881299139), the disclosure of which is incorporated herein by reference in its entirety.

Isothermal amplification approaches, such as rolling circle amplification, are also now well-described. See, e.g., Schweitzer et al., Curr. Opin. Biotechnol. 12(1):21-7 (2001); U.S. Pat. Nos. 6,235,502, 6,221,603, 6,210,884, 6,183,960, 5,854,033, 5,714,320, 5,648,245, and international patent publications WO 97/19193 and WO 00/15779, the disclosures of which are incorporated herein by reference in their entireties. Rolling circle amplification can be combined with other techniques to facilitate SNP detection. See, e.g., Lizardi et al., Nature Genet. 19(3):225-32 (1998).

As further described below, nucleic acids of the present invention, inserted into vectors that flank the nucleic acid insert with a phage promoter, such as T7, T3, or SP6 promoter, can be used to drive in vitro expression of RNA complementary to either strand of the nucleic acid of the present invention. The RNA can be used, inter alia, as a single-stranded probe, in cDNA-mRNA subtraction, or for in vitro translation.

As will be further discussed herein below, nucleic acids of the present invention that encode LCP protein or portions thereof can be used, inter alia, to express the LCP proteins or protein fragments, either alone, or as part of fusion proteins.

Expression can be from genomic nucleic acids of the present invention, or from transcript-derived nucleic acids of the present invention.

Where protein expression is effected from genomic DNA, expression will typically be effected in eukaryotic, typically mammalian, cells capable of splicing introns from the initial RNA transcript. Expression can be driven from episomal vectors, such as EBV-based vectors, or can be effected from genomic DNA integrated into a host cell chromosome. As will be more fully described below, where expression is from transcript-derived (or otherwise intron-less) nucleic acids of the present invention, expression can be effected in wide variety of prokaryotic or eukaryotic cells.

Expressed in vitro, the protein, protein fragment, or protein fusion can thereafter be isolated, to be used, inter alia, as a standard in immunoassays specific for the proteins, or protein isoforms, of the present invention; to be used as a therapeutic agent, e.g., to be administered as passive replacement therapy in individuals deficient in the proteins of the present invention, or to be administered as a vaccine; to be used for in vitro production of specific antibody, the antibody thereafter to be used, e.g., as an analytical reagent for detection and quantitation of the proteins of the present invention or to be used as an immunotherapeutic agent.

The isolated nucleic acids of the present invention can also be used to drive in vivo expression of the proteins of the present invention. In vivo expression can be driven from a vector—typically a viral vector, often a vector based upon a replication incompetent retrovirus, an adenovirus, or an adeno-associated virus (AAV)—for purpose of gene therapy. In vivo expression can also be driven from signals endogenous to the nucleic acid or from a vector, often a plasmid vector, such as pVAX1 (Invitrogen, Carlsbad Calif., USA), for purpose of “naked” nucleic acid vaccination, as further described in U.S. Pat. Nos. 5,589,466; 5,679,647; 5,804,566; 5,830,877; 5,843,913; 5,880,104; 5,958,891; 5,985,847; 6,017,897; 6,110,898; 6,204,250, the disclosures of which are incorporated herein by reference in their entireties.

The nucleic acids of the present invention can also be used for antisense inhibition of transcription or translation. See Phillips (ed.), Antisense Technology, Part B, Methods in Enzymology Vol. 314, Academic Press, Inc. (1999) (ISBN: 012182215X); Phillips (ed.), Antisense Technology, Part A, Methods in Enzymology Vol. 313, Academic Press, Inc. (1999) (ISBN: 0121822141); Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Agrawal et al. (eds.), Antisense Research and Application, Springer-Verlag New York, Inc. (1998) (ISBN: 3540638334); Lichtenstein et al. (eds.), Antisense Technology: A Practical Approach, Vol. 185, Oxford University Press, INC. (1998) (ISBN: 0199635838); Gibson (ed.), Antisense and Ribozyme Methodology: Laboratory Companion, Chapman & Hall (1997) (ISBN: 3826100794); Chadwick et al. (eds.), Oligonucleotides as Therapeutic Agents—Symposium No. 209, John Wiley & Son Ltd (1997) (ISBN: 0471972797), the disclosures of which are incorporated herein by reference in their entireties.

Nucleic acids of the present invention, particularly cDNAs of the present invention, that encode full-length human LCP protein isoforms, have additional, well-recognized, immediate, real world utility as commercial products of manufacture suitable for sale.

For example, Invitrogen Corp. (Carlsbad, Calif., USA), through its Research Genetics subsidiary, sells full length human cDNAs cloned into one of a selection of expression vectors as GeneStorm® expression-ready clones; utility is specific for the gene, since each gene is capable of being ordered separately and has a distinct catalogue number, and utility is substantial, each clone selling for $650.00 US. Similarly, Incyte Genomics (Palo Alto, Calif., USA) sells clones from public and proprietary sources in multi-well plates or individual tubes.

Nucleic acids of the present invention that include genomic regions encoding the human LCP protein, or portions thereof, have yet further utilities.

For example, genomic nucleic acids of the present invention can be used as amplification substrates, e.g. for preparation of genome-derived single exon probes of the present invention, as described above and in copending and commonly-owned U.S. patent application Ser. No. 09/864,761, filed May 23, 2001, Ser. No. 09/774,203, filed Jan. 29, 2001, and Ser. No. 09/632,366, filed Aug. 3, 2000, the disclosures of which are incorporated herein by reference in their entireties.

As another example, genomic nucleic acids of the present invention can be integrated non-homologously into the genome of somatic cells, e.g. CHO cells, COS cells, or 293 cells, with or without amplification of the insertional locus, in order, e.g., to create stable cell lines capable of producing the proteins of the present invention.

As another example, more fully described herein below, genomic nucleic acids of the present invention can be integrated nonhomologously into embryonic stem (ES) cells to create transgenic non-human animals capable of producing the proteins of the present invention.

Genomic nucleic acids of the present invention can also be used to target homologous recombination to the human LCP locus. See, e.g., U.S. Pat. Nos. 6,187,305; 6,204,061; 5,631,153; 5,627,059; 5,487,992; 5,464,764; 5,614,396; 5,527,695 and 6,063,630; and Kmiec et al. (eds.), Gene Targeting Protocols, Vol. 133, Humana Press (2000) (ISBN: 0896033600); Joyner (ed.), Gene Targeting: A Practical Approach, Oxford University Press, Inc. (2000) (ISBN: 0199637938); Sedivy et al., Gene Targeting, Oxford University Press (1998) (ISBN: 071677013X); Tymms et al. (eds.), Gene Knockout Protocols, Humana Press (2000) (ISBN: 0896035727); Mak et al. (eds.), The Gene Knockout FactsBook, Vol. 2, Academic Press, Inc. (1998) (ISBN: 0124660444); Torres et al., Laboratory Protocols for Conditional Gene Targeting, Oxford University Press (1997) (ISBN: 019963677X); Vega (ed.), Gene Targeting, CRC Press, LLC (1994) (ISBN: 084938950X), the disclosures of which are incorporated herein by reference in their entireties.

Where the genomic region includes transcription regulatory elements, homologous recombination can be used to alter the expression of LCP, both for purpose of in vitro production of LCP protein from human cells, and for purpose of gene therapy. See, e.g., U.S. Pat. Nos. 5,981,214, 6,048,524; 5,272,071.

Fragments of the nucleic acids of the present invention smaller than those typically used for homologous recombination can also be used for targeted gene correction or alteration, possibly by cellular mechanisms different from those engaged during homologous recombination.

For example, partially duplexed RNA/DNA chimeras have been shown to have utility in targeted gene correction, U.S. Pat. Nos. 5,945,339, 5,888,983, 5,871,984, 5,795,972, 5,780,296, 5,760,012, 5,756,325, 5,731,181, the disclosures of which are incorporated herein by reference in their entireties. So too have small oligonucleotides fused to triplexing domains have been shown to have utility in targeted gene correction, Culver et al., “Correction of chromosomal point mutations in human cells with bifunctional oligonucleotides,” Nature Biotechnol. 17(10):989-93 (1999), as have oligonucleotides having modified terminal bases or modified terminal internucleoside bonds, Gamper et al., Nucl. Acids Res. 28(21):4332-9 (2000), the disclosures of which are incorporated herein by reference.

The isolated nucleic acids of the present invention can also be used to provide the initial substrate for recombinant engineering of LCP protein variants having desired phenotypic improvements. Such engineering includes, for example, site-directed mutagenesis, random mutagenesis with subsequent functional screening, and more elegant schemes for recombinant evolution of proteins, as are described, inter alia, in U.S. Pat. Nos. 6,180,406; 6,165,793; 6,117,679; and 6,096,548, the disclosures of which are incorporated herein by reference in their entireties.

Nucleic acids of the present invention can be obtained by using the labeled probes of the present invention to probe nucleic acid samples, such as genomic libraries, cDNA libraries, and mRNA samples, by standard techniques. Nucleic acids of the present invention can also be obtained by amplification, using the nucleic acid primers of the present invention, as further demonstrated in Example 1, herein below. Nucleic acids of the present invention of fewer than about 100 nt can also be synthesized chemically, typically by solid phase synthesis using commercially available automated synthesizers.

“Full Length” LCP Nucleic Acids

In a first series of nucleic acid embodiments, the invention provides isolated nucleic acids that encode the entirety of the LCP proteins. As discussed above, the “full-length” nucleic acids of the present invention can be used, inter alia, to express full length LCP proteins. The full-length nucleic acids can also be used as nucleic acid probes; used as probes, the isolated nucleic acids of these embodiments will hybridize to LCP.

In a first such embodiment, the invention provides an isolated nucleic acid comprising (i) the nucleotide sequence of SEQ ID NO: 1, or (ii) the complement of (i). SEQ ID NO: 1 presents the entire cDNA of LCP1, including the 5′ untranslated (UT) region and 3′ UT.

In a second embodiment, the invention provides an isolated nucleic acid comprising (i) the nucleotide sequence of SEQ ID NOs: 2 or 1113, (ii) a degenerate variant of the nucleotide sequence of SEQ ID NOs: 2 or 1113, or (iii) the complement of (i) or (ii). SEQ ID NO: 2 and 1113 present the open reading frame (ORF) of LCP1 and LCP2.

In a third embodiment, the invention provides an isolated nucleic acid comprising (i) a nucleotide sequence that encodes a polypeptide with the amino acid sequence of SEQ ID NOs: 3 or 1114 or (ii) the complement of a nucleotide sequence that encodes a polypeptide with the amino acid sequence of SEQ ID NOs: 3 or 1114. SEQ ID NO: 3 and 1114 provides the amino acid sequences of LCP1 and LCP2.

In a fourth embodiment, the invention provides an isolated nucleic acid having a nucleotide sequence that (i) encodes a polypeptide having the sequence of SEQ ID NOs: 3 or 1114, (ii) encodes a polypeptide having the sequence of SEQ ID NOs: 3 or 1114 with conservative amino acid substitutions, or (iii) that is the complement of (i) or (ii), where SEQ ID NO: 3 and 1114 provides the amino acid sequence of LCP1 and LCP2.

Selected Partial Nucleic Acids

In a second series of nucleic acid embodiments, the invention provides isolated nucleic acids that encode select portions of LCP. As will be further discussed herein below, these “partial” nucleic acids can be used, inter alia, to express specific portions of the LCP. These “partial” nucleic acids can also be used, inter alia, as nucleic probes.

In a first such embodiment, the invention provides an isolated nucleic acid comprising (i) the nucleotide sequence of SEQ ID NO: 4, (ii) a degenerate variant of SEQ ID NO: 4, or (iii) the complement of (i) or (ii), wherein the isolated nucleic acid is no more than about 100 kb in length, typically no more than about 75 kb in length, more typically no more than about 50 kb length. SEQ ID NO: 4 encodes a novel portion of LCP. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) a nucleotide sequence that encodes SEQ ID NO: 5 or (ii) the complement of a nucleotide sequence that encodes SEQ ID NO: 5, wherein the isolated nucleic acid is no more than about 100 kb in length, typically no more than about 75 kb in length, frequently no more than about 50 kb in length. SEQ ID NO: 5 is the amino acid sequence encoded by a portion of LCP not found in any EST fragments. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) a nucleotide sequence that encodes SEQ ID NO: 5, (ii) a nucleotide sequence that encodes SEQ ID NO: 5 with conservative substititions, or (iii) the complement of (i) or (ii), wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In another such embodiment, the invention provides an isolated nucleic acid comprising (i) the nucleotide sequence of SEQ ID NO: 6, (ii) a degenerate variant of SEQ ID NO: 7, or (iii) the complement of (i) or (ii), wherein the isolated nucleic acid is no more than about 100 kb in length, typically no more than about 75 kb in length, more typically no more than about 50 kb length. SEQ ID NO: 7 encodes a novel portion of LCP. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) a nucleotide sequence that encodes SEQ ID NO: 9 or (ii) the complement of a nucleotide sequence that encodes SEQ ID NO: 9, wherein the isolated nucleic acid is no more than about 100 kb in length, typically no more than about 75 kb in length, frequently no more than about 50 kb in length. SEQ ID NO: 9 is the amino acid sequence encoded by another portion of LCP not found in any EST fragments. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) a nucleotide sequence that encodes SEQ ID NO: 9, (ii) a nucleotide sequence that encodes SEQ ID NO: 9 with conservative substititions, or (iii) the complement of (i) or (ii), wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a first such embodiment, the invention provides an isolated nucleic acid comprising (i) the nucleotide sequence of SEQ ID NO: 1115, (ii) a degenerate variant of SEQ ID NO: 1115, or (iii) the complement of (i) or (ii), wherein the isolated nucleic acid is no more than about 100 kb in length, typically no more than about 75 kb in length, more typically no more than about 50 kb length. SEQ ID NO: 1115 represents the splice junction of exons 1 and 3 of LCP1 and encodes a novel portion of LCP2. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) a nucleotide sequence that encodes SEQ ID NO: 1116 or (ii) the complement of a nucleotide sequence that encodes SEQ ID NO: 1116, wherein the isolated nucleic acid is no more than about 100 kb in length, typically no more than about 75 kb in length, frequently no more than about 50 kb in length. SEQ ID NO: 1116 is the amino acid sequence encoded by SEQ ID NO: 1115. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) a nucleotide sequence that encodes SEQ ID NO: 1116, (ii) a nucleotide sequence that encodes SEQ ID NO: 1116 with conservative substititions, or (iii) the complement of (i) or (ii), wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

Cross-Hybridizing Nucleic Acids

In another series of nucleic acid embodiments, the invention provides isolated nucleic acids that hybridize to various of the LCP nucleic acids of the present invention. These cross-hybridizing nucleic acids can be used, inter alia, as probes for, and to drive expression of, proteins that are related to LCP of the present invention as further isoforms, homologues, paralogues, or orthologues.

In a first embodiment, the invention provides an isolated nucleic acid comprising a sequence that hybridizes under high stringency conditions to a probe the nucleotide sequence of which consists of at least 17 nt, 18, 19, 20, 21, 22, 23, 24, 25, 30, 40, or 50 nt of SEQ ID NO: 4 or the complement of SEQ ID NO: 4, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under moderate stringency conditions to a probe the nucleotide sequence of which consists of at least 17 nt, 18, 19, 20, 21, 22, 23, 24, 25, 30, 40, or 50 nt of SEQ ID NO: 4 or the complement of SEQ ID NO: 4, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under high stringency conditions to a hybridization probe the nucleotide sequence of which (i) encodes a polypeptide having the sequence of SEQ ID NO: 5, (ii) encodes a polypeptide having the sequence of SEQ ID NO: 5 with conservative amino acid substitutions, or (iii) is the complement of (i) or (ii), wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In another embodiment, the invention provides an isolated nucleic acid comprising a sequence that hybridizes under high stringency conditions to a probe the nucleotide sequence of which consists of at least 17 nt, 18, 19, 20, 21, 22, 23, 24, 25, 30, 40, or 50 nt of SEQ ID NO: 6 or the complement of SEQ ID NO: 6, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under moderate stringency conditions to a probe the nucleotide sequence of which consists of at least 17 nt, 18, 19, 20, 21, 22, 23, 24, 25, 30, 40, or 50 nt of SEQ ID NO: 6 or the complement of SEQ ID NO: 6, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under high stringency conditions to a hybridization probe the nucleotide sequence of which (i) encodes a polypeptide having the sequence of SEQ ID NO: 9, (ii) encodes a polypeptide having the sequence of SEQ ID NO: 9 with conservative amino acid substitutions, or (iii) is the complement of (i) or (ii), wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under high stringency conditions to a probe the nucleotide sequence of which consists of at least 17 nt, 18, 19, 20, 21, 22, 23, 24, 25, 30, 40, or 50 nt of SEQ ID NO: 1115 or the complement of SEQ ID NO: 1115, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under moderate stringency conditions to a probe the nucleotide sequence of which consists of at least 17 nt, 18, 19, 20, 21, 22, 23, 24, 25, 30, 40, or 50 nt of SEQ ID NO: 1115 or the complement of SEQ ID NO: 1115, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under high stringency conditions to a hybridization probe the nucleotide sequence of which (i) encodes a polypeptide having the sequence of SEQ ID NO: 1116, (ii) encodes a polypeptide having the sequence of SEQ ID NO: 1116 with conservative amino acid substitutions, or (iii) is the complement of (i) or (ii), wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

Particularly Useful Nucleic Acids

Particularly useful among the above-described nucleic acids are those that are expressed, or the complement of which are expressed, in adrenal, adult liver, bone marrow, brain, fetal liver, heart, kidney, lung, placenta, skeletal muscle, colon and prostate as well as a cell line hela.

Also particularly useful among the above-described nucleic acids are those that encode, or the complement of which encode, a polypeptide that plays a potential therapeutic as well as diagnostic role for neurological and developmental disorders, as well as diseases involving cell-cell adhesion process.

Other particularly useful embodiments of the nucleic acids above-described are those that encode, or the complement of which encode, a polypeptide having any or all of CUB, LCCL, and discoidin/FA8C domains.

Nucleic Acid Fragments

In another series of nucleic acid embodiments, the invention provides fragments of various of the isolated nucleic acids of the present invention which prove useful, inter alia, as nucleic acid probes, as amplification primers, and to direct expression or synthesis of epitopic or immunogenic protein fragments.

In a first such embodiment, the invention provides an isolated nucleic acid comprising at least 17 nucleotides, 18 nucleotides, 20 nucleotides, 24 nucleotides, or 25 nucleotides of (i) SEQ ID NO: 4, (ii) a degenerate variant of SEQ ID NO: 4, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

The invention also provides an isolated nucleic acid comprising (i) a nucleotide sequence that encodes a peptide of at least 8 contiguous amino acids of SEQ ID NO: 5, (ii) a nucleotide sequence that encodes a peptide of at least 15 contiguous amino acids of SEQ ID NO: 5, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

The invention also provides an isolated nucleic acid comprising a nucleotide sequence that encodes (i) a polypeptide having the sequence of at least 8 contiguous amino acids of SEQ ID NO: 5 with conservative amino acid substitutions, (ii) a polypeptide having the sequence of at least 15 contiguous amino acids of SEQ ID NO: 5 with conservative amino acid substitutions, (iii) a polypeptide having the sequence of at least 8 contiguous amino acids of SEQ ID NO: 5 with moderately conservative substitutions, (iv) a polypeptide having the sequence of at last 15 congiuous amino acids of SEQ ID NO: 5 with moderately conservative substitutions, or (v) the complement of any of (i)-(iv), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

In another such embodiment, the invention provides an isolated nucleic acid comprising at least 17 nucleotides, 18 nucleotides, 20 nucleotides, 24 nucleotides, or 25 nucleotides of (i) SEQ ID NO: 6, (ii) a degenerate variant of SEQ ID NO: 7, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

The invention also provides an isolated nucleic acid comprising (i) a nucleotide sequence that encodes a peptide of at least 8 contiguous amino acids of SEQ ID NO: 9, (ii) a nucleotide sequence that encodes a peptide of at least 15 contiguous amino acids of SEQ ID NO: 9, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

The invention also provides an isolated nucleic acid comprising a nucleotide sequence that encodes (i) a polypeptide having the sequence of at least 8 contiguous amino acids of SEQ ID NO: 9 with conservative amino acid substitutions, (ii) a polypeptide having the sequence of at least 15 contiguous amino acids of SEQ ID NO: 9 with conservative amino acid substitutions, (iii) a polypeptide having the sequence of at least 8 contiguous amino acids of SEQ ID NO: 9 with moderately conservative substitutions, (iv) a polypeptide having the sequence of at last 15 congiuous amino acids of SEQ ID NO: 9 with moderately conservative substitutions, or (v) the complement of any of (i)-(iv), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

In another such embodiment, the invention provides an isolated nucleic acid comprising at least 17 nucleotides, 18 nucleotides, 20 nucleotides, 24 nucleotides, or 25 nucleotides of (i) SEQ ID NO: 1115, (ii) a degenerate variant of SEQ ID NO: 1115, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

The invention also provides an isolated nucleic acid comprising (i) a nucleotide sequence that encodes a peptide of at least 8 contiguous amino acids of SEQ ID NO: 1116, (ii) a nucleotide sequence that encodes a peptide of at least 15 contiguous amino acids of SEQ ID NO: 1116, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

The invention also provides an isolated nucleic acid comprising a nucleotide sequence that encodes (i) a polypeptide having the sequence of at least 8 contiguous amino acids of SEQ ID NO: 1116 with conservative amino acid substitutions, (ii) a polypeptide having the sequence of at least 15 contiguous amino acids of SEQ ID NO: 1116 with conservative amino acid substitutions, (iii) a polypeptide having the sequence of at least 8 contiguous amino acids of SEQ ID NO: 1116 with moderately conservative substitutions, (iv) a polypeptide having the sequence of at last 15 congiuous amino acids of SEQ ID NO: 1116 with moderately conservative substitutions, or (v) the complement of any of (i)-(iv), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

Single Exon Probes

The invention further provides genome-derived single exon probes having portions of no more than one exon of the LCP gene. As further described in commonly owned and copending U.S. patent application Ser. No. 09/632,366, filed Aug. 3, 2000 (“Methods and Apparatus for High Throughput Detection and Characterization of alternatively Spliced Genes”), the disclosure of which is incorporated herein by reference in its entirety, such single exon probes have particular utility in identifying and characterizing splice variants. In particular, such single exon probes are useful for identifying and discriminating the expression of distinct isoforms of LCP.

In a first embodiment, the invention provides an isolated nucleic acid comprising a nucleotide sequence of no more than one portion of SEQ ID NOs: 10-25 or the complement of SEQ ID NOs: 10-25, wherein the portion comprises at least 17 contiguous nucleotides, 18 contiguous nucleotides, 20 contiguous nucleotides, 24 contiguous nucleotides, 25 contiguous nucleotides, or 50 contiguous nucleotides of any one of SEQ ID NOs: 10-25, or their complement. In a further embodiment, the exonic portion comprises the entirety of the referenced SEQ ID NO: or its complement.

In other embodiments, the invention provides isolated single exon probes having the nucleotide sequence of any one of SEQ ID NOs: 26-41.

Transcription Control Nucleic Acids

In another aspect, the present invention provides genome-derived isolated nucleic acids that include nucleic acid sequence elements that control transcription of the LCP gene. These nucleic acids can be used, inter alia, to drive expression of heterologous coding regions in recombinant constructs, thus conferring upon such heterologous coding regions the expression pattern of the native LCP gene. These nucleic acids can also be used, conversely, to target heterologous transcription control elements to the LCP genomic locus, altering the expression pattern of the LCP gene itself.

In a first such embodiment, the invention provides an isolated nucleic acid comprising the nucleotide sequence of SEQ ID NO: 42 or its complement, wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

In another embodiment, the invention provides an isolated nucleic acid comprising at least 17, 18, 20, 24, or 25 nucleotides of the sequence of SEQ ID NO: 42 or its complement, wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

Vectors and Host Cells

In another aspect, the present invention provides vectors that comprise one or more of the isolated nucleic acids of the present invention, and host cells in which such vectors have been introduced.

The vectors can be used, inter alia, for propagating the nucleic acids of the present invention in host cells (cloning vectors), for shuttling the nucleic acids of the present invention between host cells derived from disparate organisms (shuttle vectors), for inserting the nucleic acids of the present invention into host cell chromosomes (insertion vectors), for expressing sense or antisense RNA transcripts of the nucleic acids of the present invention in vitro or within a host cell, and for expressing polypeptides encoded by the nucleic acids of the present invention, alone or as fusions to heterologous polypeptides. Vectors of the present invention will often be suitable for several such uses.

Vectors are by now well-known in the art, and are described, inter alia, in Jones et al. (eds.), Vectors: Cloning Applications: Essential Techniques (Essential Techniques Series), John Wiley & Son Ltd 1998 (ISBN: 047196266X); Jones et al. (eds.), Vectors: Expression Systems: Essential Techniques (Essential Techniques Series), John Wiley & Son Ltd, 1998 (ISBN:0471962678); Gacesa et al., Vectors: Essential Data, John Wiley & Sons, 1995 (ISBN: 0471948411); Cid-Arregui (eds.), Viral Vectors: Basic Science and Gene Therapy, Eaton Publishing Co., 2000 (ISBN: 188129935X); Sambrook et al., Molecular Cloning: A Laboratory Manual (3^{rd} ed.), Cold Spring Harbor Laboratory Press, 2001 (ISBN: 0879695773); Ausubel et al. (eds.), Short Protocols in Molecular Biology: A Compendium of Methods from Current Protocols in Molecular Biology (4^{th} ed.), John Wiley & Sons, 1999 (ISBN: 047132938X), the disclosures of which are incorporated herein by reference in their entireties. Furthermore, an enormous variety of vectors are available commercially. Use of existing vectors and modifications thereof being well within the skill in the art, only basic features need be described here.

Typically, vectors are derived from virus, plasmid, prokaryotic or eukaryotic chromosomal elements, or some combination thereof, and include at least one origin of replication, at least one site for insertion of heterologous nucleic acid, typically in the form of a polylinker with multiple, tightly clustered, single cutting restriction sites, and at least one selectable marker, although some integrative vectors will lack an origin that is functional in the host to be chromosomally modified, and some vectors will lack selectable markers. Vectors of the present invention will further include at least one nucleic acid of the present invention inserted into the vector in at least one location.

Where present, the origin of replication and selectable markers are chosen based upon the desired host cell or host cells; the host cells, in turn, are selected based upon the desired application.

For example, prokaryotic cells, typically E. coli, are typically chosen for cloning. In such case, vector replication is predicated on the replication strategies of coliform-infecting phage—such as phage lambda, M13, T7, T3 and P1—or on the replication origin of autonomously replicating episomes, notably the ColE1 plasmid and later derivatives, including pBR322 and the pUC series plasmids. Where E. coli is used as host, selectable markers are, analogously, chosen for selectivity in gram negative bacteria: e.g., typical markers confer resistance to antibiotics, such as ampicillin, tetracycline, chloramphenicol, kanamycin, streptomycin, zeocin; auxotrophic markers can also be used.

As another example, yeast cells, typically S. cerevisiae, are chosen, inter alia, for eukaryotic genetic studies, due to the ease of targeting genetic changes by homologous recombination and to the ready ability to complement genetic defects using recombinantly expressed proteins, for identification of interacting protein components, e.g. through use of a two-hybrid system, and for protein expression. Vectors of the present invention for use in yeast will typically, but not invariably, contain an origin of replication suitable for use in yeast and a selectable marker that is functional in yeast.

Integrative YIp vectors do not replicate autonomously, but integrate, typically in single copy, into the yeast genome at low frequencies and thus replicate as part of the host cell chromosome; these vectors lack an origin of replication that is functional in yeast, although they typically have at least one origin of replication suitable for propagation of the vector in bacterial cells. YEp vectors, in contrast, replicate episomally and autonomously due to presence of the yeast 2 micron plasmid origin (2 μm ori). The YCp yeast centromere plasmid vectors are autonomously replicating vectors containing centromere sequences, CEN, and autonomously replicating sequences, ARS; the ARS sequences are believed to correspond to the natural replication origins of yeast chromosomes. YACs are based on yeast linear plasmids, denoted YLp, containing homologous or heterologous DNA sequences that function as telomeres (TEL) in vivo, as well as containing yeast ARS (origins of replication) and CEN (centromeres) segments.

Selectable markers in yeast vectors include a variety of auxotrophic markers, the most common of which are (in Saccharomyces cerevisiae) URA3, HIS3, LEU2, TRP1 and LYS2, which complement specific auxotrophic mutations, such as ura3-52, his3-D1, leu2-D1, trp1-D1 and lys2-201. The URA3 and LYS2 yeast genes further permit negative selection based on specific inhibitors, 5-fluoro-orotic acid (FOA) and α-aminoadipic acid (αAA), respectively, that prevent growth of the prototrophic strains but allows growth of the ura3 and lys2 mutants, respectively. Other selectable markers confer resistance to, e.g., zeocin.

As yet another example, insect cells are often chosen for high efficiency protein expression. Where the host cells are from Spodoptera frugiperda—e.g., Sf9 and Sf21 cell lines, and expresSF™ cells (Protein Sciences Corp., Meriden, Conn., USA)—the vector replicative strategy is typically based upon the baculovirus life cycle. Typically, baculovirus transfer vectors are used to replace the wild-type AcMNPV polyhedrin gene with a heterologous gene of interest. Sequences that flank the polyhedrin gene in the wild-type genome are positioned 5′ and 3′ of the expression cassette on the transfer vectors. Following cotransfection with AcMNPV DNA, a homologous recombination event occurs between these sequences resulting in a recombinant virus carrying the gene of interest and the polyhedrin or p10 promoter. Selection can be based upon visual screening for lacZ fusion activity.

As yet another example, mammalian cells are often chosen for expression of proteins intended as pharmaceutical agents, and are also chosen as host cells for screening of potential agonist and antagonists of a protein or a physiological pathway.

Where mammalian cells are chosen as host cells, vectors intended for autonomous extrachromosomal replication will typically include a viral origin, such as the SV40 origin (for replication in cell lines expressing the large T-antigen, such as COS1 and COS7 cells), the papillomavirus origin, or the EBV origin for long term episomal replication (for use, e.g., in 293-EBNA cells, which constitutively express the EBV EBNA-1 gene product and adenovirus E1A). Vectors intended for integration, and thus replication as part of the mammalian chromosome, can, but need not, include an origin of replication functional in mammalian cells, such as the SV40 origin. Vectors based upon viruses, such as adenovirus, adeno-associated virus, vaccinia virus, and various mammalian retroviruses, will typically replicate according to the viral replicative strategy.

Selectable markers for use in mammalian cells include resistance to neomycin (G418), blasticidin, hygromycin and to zeocin, and selection based upon the purine salvage pathway using HAT medium.

Plant cells can also be used for expression, with the vector replicon typically derived from a plant virus (e.g., cauliflower mosaic virus, CaMV; tobacco mosaic virus, TMV) and selectable markers chosen for suitability in plants.

For propagation of nucleic acids of the present invention that are larger than can readily be accomodated in vectors derived from plasmids or virus, the invention further provides artificial chromosomes—BACs, YACs, PACs, and HACs—that comprise LCP nucleic acids, often genomic nucleic acids.

The BAC system is based on the well-characterized E. coli F-factor, a low copy plasmid that exists in a supercoiled circular form in host cells. The structural features of the F-factor allow stable maintenance of individual human DNA clones as well as easy manipulation of the cloned DNA. See Shizuya et al., Keio J. Med. 50(1):26-30 (2001); Shizuya et al., Proc. Natl. Acad. Sci. USA 89(18):8794-7 (1992).

YACs are based on yeast linear plasmids, denoted YLp, containing homologous or heterologous DNA sequences that function as telomeres (TEL) in vivo, as well as containing yeast ARS (origins of replication) and CEN (centromeres) segments.

HACs are human artifical chromosomes. Kuroiwa et al., Nature Biotechnol. 18(10):1086-90 (2000); Henning et al., Proc. Natl. Acad. Sci. USA 96(2):592-7 (1999); Harrington et al., Nature Genet. 15(4):345-55 (1997). In one version, long synthetic arrays of alpha satellite DNA are combined with telomeric DNA and genomic DNA to generate linear microchromosomes that are mitotically and cytogenetically stable in the absence of selection.

PACs are P1-derived artificial chromosomes. Sternberg, Proc. Natl. Acad. Sci. USA 87(l):103-7 (1990); Sternberg et al., New Biol. 2(2):151-62 (1990); Pierce et al., Proc. Natl Acad. Sci. USA 89(6):2056-60 (1992).

Vectors of the present invention will also often include elements that permit in vitro transcription of RNA from the inserted heterologous nucleic acid. Such vectors typically include a phage promoter, such as that from T7, T3, or SP6, flanking the nucleic acid insert. Often two different such promoters flank the inserted nucleic acid, permitting separate in vitro production of both sense and antisense strands.

Expression vectors of the present invention—that is, those vectors that will drive expression of polypeptides from the inserted heterologous nucleic acid—will often include a variety of other genetic elements operatively linked to the protein-encoding heterologous nucleic acid insert, typically genetic elements that drive transcription, such as promoters and enhancer elements, those that facilitate RNA processing, such as transcription termination and/or polyadenylation signals, and those that facilitate translation, such as ribosomal consensus sequences.

For example, vectors for expressing proteins of the present invention in prokaryotic cells, typically E. coli, will include a promoter, often a phage promoter, such as phage lambda pL promoter, the trc promoter, a hybrid derived from the trp and lac promoters, the bacteriophage T7 promoter (in E. coli cells engineered to express the T7 polymerase), or the araBAD operon. Often, such prokaryotic expression vectors will further include transcription terminators, such as the aspA terminator, and elements that facilitate translation, such as a consensus ribosome binding site and translation termination codon, Schomer et al., Proc. Natl. Acad. Sci. USA 83:8506-8510 (1986).

As another example, vectors for expressing proteins of the present invention in yeast cells, typically S. cerevisiae, will include a yeast promoter, such as the CYC1 promoter, the GAL1 promoter, ADH1 promoter, or the GPD promoter, and will typically have elements that facilitate transcription termination, such as the transcription termination signals from the CYC1 or ADH1 gene.

As another example, vectors for expressing proteins of the present invention in mammalian cells will include a promoter active in mammalian cells. Such promoters are often drawn from mammalian viruses—such as the enhancer-promoter sequences from the immediate early gene of the human cytomegalovirus (CMV), the enhancer-promoter sequences from the Rous sarcoma virus long terminal repeat (RSV LTR), and the enhancer-promoter from SV40. Often, expression is enhanced by incorporation of polyadenylation sites, such as the late SV40 polyadenylation site and the polyadenylation signal and transcription termination sequences from the bovine growth hormone (BGH) gene, and ribosome binding sites. Furthermore, vectors can include introns, such as intron II of rabbit β-globin gene and the SV40 splice elements.

Vector-drive protein expression can be constitutive or inducible.

Inducible vectors include either naturally inducible promoters, such as the trc promoter, which is regulated by the lac operon, and the pL promoter, which is regulated by tryptophan, the MMTV-LTR promoter, which is inducible by dexamethasone, or can contain synthetic promoters and/or additional elements that confer inducible control on adjacent promoters. Examples of inducible synthetic promoters are the hybrid Plac/ara-1 promoter and the PLtetO-1 promoter. The PltetO-1 promoter takes advantage of the high expression levels from the PL promoter of phage lambda, but replaces the lambda repressor sites with two copies of operator 2 of the Tn10 tetracycline resistance operon, causing this promoter to be tightly repressed by the Tet repressor protein and induced in response to tetracycline (Tc) and Tc derivatives such as anhydrotetracycline.

As another example of inducible elements, hormone response elements, such as the glucocorticoid response element (GRE) and the estrogen response element (ERE), can confer hormone inducibility where vectors are used for expression in cells having the respective hormone receptors. To reduce background levels of expression, elements responsive to ecdysone, an insect hormone, can be used instead, with coexpression of the ecdysone receptor.

Expression vectors can be designed to fuse the expressed polypeptide to small protein tags that facilitate purification and/or visualization.

For example, proteins of the present invention can be expressed with a polyhistidine tag that facilitates purification of the fusion protein by immobilized metal affinity chromatography, for example using NiNTA resin (Qiagen Inc., Valencia, Calif., USA) or TALON™ resin (cobalt immobilized affinity chromatography medium, Clontech Labs, Palo Alto, Calif., USA). As another example, the fusion protein can include a chitin-binding tag and self-excising intein, permitting chitin-based purification with self-removal of the fused tag (IMPACT™ system, New England Biolabs, Inc., Beverley, Mass., USA). Alternatively, the fusion protein can include a calmodulin-binding peptide tag, permitting purification by calmodulin affinity resin (Stratagene, La Jolla, Calif., USA), or a specifically excisable fragment of the biotin carboxylase carrier protein, permitting purification of in vivo biotinylated protein using an avidin resin and subsequent tag removal (Promega, Madison, Wis., USA). As another useful alternative, the proteins of the present invention can be expressed as a fusion to glutathione-S-transferase, the affinity and specificity of binding to glutathione permitting purification using glutathione affinity resins, such as Glutathione-Superflow Resin (Clontech Laboratories, Palo Alto, Calif., USA), with subsequent elution with free glutathione.

Other tags include, for example, the Xpress epitope, detectable by anti-Xpress antibody (Invitrogen, Carlsbad, Calif., USA), a myc tag, detectable by anti-myc tag antibody, the V5 epitope, detectable by anti-V5 antibody (Invitrogen, Carlsbad, Calif., USA), FLAG® epitope, detectable by anti-FLAG antibody (Stratagene, La Jolla, Calif., USA), and the HA epitope.

For secretion of expressed proteins, vectors can include appropriate sequences that encode secretion signals, such as leader peptides. For example, the pSecTag2 vectors (Invitrogen, Carlsbad, Calif., USA) are 5.2 kb mammalian expression vectors that carry the secretion signal from the V-J2-C region of the mouse Ig kappa-chain for efficient secretion of recombinant proteins from a variety of mammalian cell lines.

Expression vectors can also be designed to fuse proteins encoded by the heterologous nucleic acid insert to polypeptides larger than purification and/or identification tags. Useful protein fusions include those that permit display of the encoded protein on the surface of a phage or cell, fusions to intrinsically fluorescent proteins, such as those that have a green fluorescent protein (GFP)-like chromophore, fusions to the IgG Fc region, and fusions for use in two hybrid systems.

Vectors for phage display fuse the encoded polypeptide to, e.g., the gene III protein (pIII) or gene VIII protein (pVIII) for display on the surface of filamentous phage, such as M13. See Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, San Diego: Academic Press, Inc., 1996; Abelson et al. (eds.), Combinatorial Chemistry, Methods in Enzymology vol. 267, Academic Press (May 1996).

Vectors for yeast display, e.g. the pYD1 yeast display vector (Invitrogen, Carlsbad, Calif., USA), use the a-agglutinin yeast adhesion receptor to display recombinant protein on the surface of S. cerevisiae. Vectors for mammalian display, e.g., the pDisplay™ vector (Invitrogen, Carlsbad, Calif., USA), target recombinant proteins using an N-terminal cell surface targeting signal and a C-terminal transmembrane anchoring domain of platelet derived growth factor receptor.

A wide variety of vectors now exist that fuse proteins encoded by heterologous nucleic acids to the chromophore of the substrate-independent, intrinsically fluorescent green fluorescent protein from Aequorea victoria (“GFP”) and its variants. These proteins are intrinsically fluorescent: the GFP-like chromophore is entirely encoded by its amino acid sequence and can fluoresce without requirement for cofactor or substrate.

Structurally, the GFP-like chromophore comprises an 11-stranded β-barrel (β-can) with a central α-helix, the central α-helix having a conjugated π-resonance system that includes two aromatic ring systems and the bridge between them. The π-resonance system is created by autocatalytic cyclization among amino acids; cyclization proceeds through an imidazolinone intermediate, with subsequent dehydrogenation by molecular oxygen at the Cα-Cβ bond of a participating tyrosine.

The GFP-like chromophore can be selected from GFP-like chromophores found in naturally occurring proteins, such as A. victoria GFP (GenBank accession number AAA27721), Renilla reniformis GFP, FP583 (GenBank accession no. AF168419) (DsRed), FP593 (AF272711), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. Li et al., “Deletions of the Aequorea Victoria Green Fluorescent Protein Define the Minimal Domain Required for Fluorescence,” J. Biol. Chem. 272:28545-28549 (1997).

Alternatively, the GFP-like chromophore can be selected from GFP-like chromophores modified from those found in nature. Typically, such modifications are made to improve recombinant production in heterologous expression systems (with or without change in protein sequence), to alter the excitation and/or emission spectra of the native protein, to facilitate purification, to facilitate or as a consequence of cloning, or are a fortuitous consequence of research investigation.

The methods for engineering such modified GFP-like chromophores and testing them for fluorescence activity, both alone and as part of protein fusions, are well-known in the art. Early results of these efforts are reviewed in Heim et al., Curr. Biol. 6:178-182 (1996), incorporated herein by reference in its entirety; a more recent review, with tabulation of useful mutations, is found in Palm et al., “Spectral Variants of Green Fluorescent Protein,” in Green Fluorescent Proteins, Conn (ed.), Methods Enzymol. vol. 302, pp. 378-394 (1999), incorporated herein by reference in its entirety. A variety of such modified chromophores are now commercially available and can readily be used in the fusion proteins of the present invention.

For example, EGFP (“enhanced GFP”), Cormack et al., Gene 173:33-38 (1996); U.S. Pat. Nos. 6,090,919 and 5,804,387, is a red-shifted, human codon-optimized variant of GFP that has been engineered for brighter fluorescence, higher expression in mammalian cells, and for an excitation spectrum optimized for use in flow cytometers. EGFP can usefully contribute a GFP-like chromophore to the fusion proteins of the present invention. A variety of EGFP vectors, both plasmid and viral, are available commercially (Clontech Labs, Palo Alto, Calif., USA), including vectors for bacterial expression, vectors for N-terminal protein fusion expression, vectors for expression of C-terminal protein fusions, and for bicistronic expression.

Toward the other end of the emission spectrum, EBFP (“enhanced blue fluorescent protein”) and BFP2 contain four amino acid substitutions that shift the emission from green to blue, enhance the brightness of fluorescence and improve solubility of the protein, Heim et al., Curr. Biol. 6:178-182 (1996); Cormack et al., Gene 173:33-38 (1996). EBFP is optimized for expression in mammalian cells whereas BFP2, which retains the original jellyfish codons, can be expressed in bacteria; as is further discussed below, the host cell of production does not affect the utility of the resulting fusion protein. The GFP-like chromophores from EBFP and BFP2 can usefully be included in the fusion proteins of the present invention, and vectors containing these blue-shifted variants are available from Clontech Labs (Palo Alto, Calif., USA).

Analogously, EYFP (“enhanced yellow fluorescent protein”), also available from Clontech Labs, contains four amino acid substitutions, different from EBFP, Ormb et al., Science 273:1392-1395 (1996), that shift the emission from green to yellowish-green. Citrine, an improved yellow fluorescent protein mutant, is described in Heikal et al., Proc. Natl. Acad. Sci. USA 97:11996-12001 (2000). ECFP (“enhanced cyan fluorescent protein”) (Clontech Labs, Palo Alto, Calif., USA) contains six amino acid substitutions, one of which shifts the emission spectrum from green to cyan. Heim et al., Curr. Biol. 6:178-182 (1996); Miyawaki et al., Nature 388:882-887 (1997). The GFP-like chromophore of each of these GFP variants can usefully be included in the fusion proteins of the present invention.

The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048, the disclosures of which are incorporated herein by reference in their entireties. See also Conn (ed.), Green Fluorescent Protein, Methods in Enzymol. Vol. 302, pp 378-394 (1999), incorporated herein by reference in its entirety. A variety of such modified chromophores are now commercially available and can readily be used in the fusion proteins of the present invention.

Fusions to the IgG Fc region increase serum half life of protein pharmaceutical products through interaction with the FcRn receptor (also denominated the FcRp receptor and the Brambell receptor, FcRb), further described in international patent application nos. WO 97/43316, WO 97/34631, WO 96/32478, WO 96/18412.

For long-term, high-yield recombinant production of the proteins, protein fusions, and protein fragments of the present invention, stable expression is particularly useful.

Stable expression is readily achieved by integration into the host cell genome of vectors having selectable markers, followed by selection for integrants.

For example, the pUB6/V5-His A, B, and C vectors (Invitrogen, Carlsbad, Calif., USA) are designed for high-level stable expression of heterologous proteins in a wide range of mammalian tissue types and cell lines. pUB6/V5-His uses the promoter/enhancer sequence from the human ubiquitin C gene to drive expression of recombinant proteins: expression levels in 293, CHO, and NIH3T3 cells are comparable to levels from the CMV and human EF-1a promoters. The bsd gene permits rapid selection of stably transfected mammalian cells with the potent antibiotic blasticidin.

Replication incompetent retroviral vectors, typically derived from Moloney murine leukemia virus, prove particularly useful for creating stable transfectants having integrated provirus. The highly efficient transduction machinery of retroviruses, coupled with the availability of a variety of packaging cell lines—such as RetroPack™ PT 67, EcoPack2™-293, AmphoPack-293, GP2-293 cell lines (all available from Clontech Laboratories, Palo Alto, Calif., USA)—allow a wide host range to be infected with high efficiency; varying the multiplicity of infection readily adjusts the copy number of the integrated provirus. Retroviral vectors are available with a variety of selectable markers, such as resistance to neomycin, hygromycin, and puromycin, permitting ready selection of stable integrants.

The present invention further includes host cells comprising the vectors of the present invention, either present episomally within the cell or integrated, in whole or in part, into the host cell chromosome.

Among other considerations, some of which are described above, a host cell strain may be chosen for its ability to process the expressed protein in the desired fashion. Such post-translational modifications of the polypeptide include, but are not limited to, acetylation, carboxylation, glycosylation, phosphorylation, lipidation, and acylation, and it is an aspect of the present invention to provide LCP proteins with such post-translational modifications.

As noted earlier, host cells can be prokaryotic or eukaryotic. Representative examples of appropriate host cells include, but are not limited to, bacterial cells, such as E. coli, Caulobacter crescentus, Streptomyces species, and Salmonella typhimurium; yeast cells, such as Saccharomyces cerevisiae, Schizosaccharomyces pombe, Pichia pastoris, Pichia methanolica; insect cell lines, such as those from Spodoptera frugiperda—e.g., Sf9 and Sf21 cell lines, and expresSF™ cells (Protein Sciences Corp., Meriden, Conn., USA)—Drosophila S2 cells, and Trichoplusia ni High Five® Cells (Invitrogen, Carlsbad, Calif., USA); and mammalian cells. Typical mammalian cells include COS1 and COS7 cells, chinese hamster ovary (CHO) cells, NIH 3T3 cells, 293 cells, HEPG2 cells, HeLa cells, L cells, murine ES cell lines (e.g., from strains 129/SV, C57/BL6, DBA-1, 129/SVJ), K562, Jurkat cells, and BW5147. Other mammalian cell lines are well known and readily available from the American Type Culture Collection (ATCC) (Manassas, Va., USA) and the National Institute of General medical Sciences (NIGMS) Human Genetic Cell Repository at the Coriell Cell Repositories (Camden, N.J., USA).

Methods for introducing the vectors and nucleic acids of the present invention into the host cells are well known in the art; the choice of technique will depend primarily upon the specific vector to be introduced and the host cell chosen.

For example, phage lambda vectors will typically be packaged using a packaging extract (e.g., Gigapack® packaging extract, Stratagene, La Jolla, Calif., USA), and the packaged virus used to infect E. coli. Plasmid vectors will typically be introduced into chemically competent or electrocompetent bacterial cells.

E. coli cells can be rendered chemically competent by treatment, e.g., with CaCl₂, or a solution of Mg²⁺, Mn²⁺, Ca²⁺, Rb+ or K+, dimethyl sulfoxide, dithiothreitol, and hexamine cobalt (III), Hanahan, J. Mol. Biol. 166(4):557-80 (1983), and vectors introduced by heat shock. A wide variety of chemically competent strains are also available commercially (e.g., Epicurian Coli® XL10-Gold® Ultracompetent Cells (Stratagene, La Jolla, Calif., USA); DH5a competent cells (Clontech Laboratories, Palo Alto, Calif., USA); TOP10 Chemically Competent E. coli Kit (Invitrogen, Carlsbad, Calif., USA)).

Bacterial cells can be rendered electrocompetent—that is, competent to take up exogenous DNA by electroporation—by various pre-pulse treatments; vectors are introduced by electroporation followed by subsequent outgrowth in selected media. An extensive series of protocols is provided online in Electroprotocols (BioRad, Richmond, Calif., USA) (http://www.bio-rad.com/LifeScience/pdf/New_Gene_Pulser.pdf).

Vectors can be introduced into yeast cells by spheroplasting, treatment with lithium salts, electroporation, or protoplast fusion.

Spheroplasts are prepared by the action of hydrolytic enzymes—a snail-gut extract, usually denoted Glusulase, or Zymolyase, an enzyme from Arthrobacter luteus—to remove portions of the cell wall in the presence of osmotic stabilizers, typically 1 M sorbitol. DNA is added to the spheroplasts, and the mixture is co-precipitated with a solution of polyethylene glycol (PEG) and Ca²⁺. Subsequently, the cells are resuspended in a solution of sorbitol, mixed with molten agar and then layered on the surface of a selective plate containing sorbitol. For lithium-mediated transformation, yeast cells are treated with lithium acetate, which apparently permeabilizes the cell wall, DNA is added and the cells are co-precipitated with PEG. The cells are exposed to a brief heat shock, washed free of PEG and lithium acetate, and subsequently spread on plates containing ordinary selective medium. Increased frequencies of transformation are obtained by using specially-prepared single-stranded carrier DNA and certain organic solvents. Schiestl et al., Curr. Genet. 16(5-6):339-46 (1989). For electroporation, freshly-grown yeast cultures are typically washed, suspended in an osmotic protectant, such as sorbitol, mixed with DNA, and the cell suspension pulsed in an electroporation device. Subsequently, the cells are spread on the surface of plates containing selective media. Becker et al., Methods Enzymol. 194:182-7 (1991). The efficiency of transformation by electroporation can be increased over 100-fold by using PEG, single-stranded carrier DNA and cells that are in late log-phase of growth. Larger constructs, such as YACs, can be introduced by protoplast fusion.

Mammalian and insect cells can be directly infected by packaged viral vectors, or transfected by chemical or electrical means.

For chemical transfection, DNA can be coprecipitated with CaPO₄ or introduced using liposomal and nonliposomal lipid-based agents. Commercial kits are available for CaPO₄ transfection (CalPhos™ Mammalian Transfection Kit, Clontech Laboratories, Palo Alto, Calif., USA), and lipid-mediated transfection can be practiced using commercial reagents, such as LIPOFECTAMINE™ 2000, LIPOFECTAMINE™ Reagent, CELLFECTIN® Reagent, and LIPOFECTIN® Reagent (Invitrogen, Carlsbad, Calif., USA), DOTAP Liposomal Transfection Reagent, FuGENE 6, X-tremeGENE Q2, DOSPER, (Roche Molecular Biochemicals, Indianapolis, Ind. USA), Effectene™, PolyFect®, Superfect® (Qiagen, Inc., Valencia, Calif., USA). Protocols for electroporating mammalian cells can be found online in Electroprotocols (Bio-Rad, Richmond, Calif., USA) (http://www.bio-rad.com/LifeScience/pdf/New_Gene_Pulser.pdf). See also, Norton et al. (eds.), Gene Transfer Methods: Introducing DNA into Living Cells and Organisms, BioTechniques Books, Eaton Publishing Co. (2000) (ISBN 1-881299-34-1), incorporated herein by reference in its entirety.

Other transfection techniques include transfection by particle embardment. See, e.g., Cheng et al., Proc. Natl. Acad. Sci. USA 90(10):4455-9 (1993); Yang et al., Proc. Natl. Acad. Sci. USA 87(24):9568-72 (1990).

Proteins

In another aspect, the present invention provides LCP proteins, various fragments thereof suitable for use as antigens (e.g., for epitope mapping) and for use as immunogens (e.g., for raising antibodies or as vaccines), fusions of LCP polypeptides and fragments to heterologous polypeptides, and conjugates of the proteins, fragments, and fusions of the present invention to other moieties (e.g., to carrier proteins, to fluorophores).

FIGS. 3 and 4 presents the predicted amino acid sequences encoded by the LCP1 and LCP2 cDNA clones. The amino acid sequence is further presented, respectively, in SEQ ID NOs: 3 and 1114.

Unless otherwise indicated, amino acid sequences of the proteins of the present invention were determined as a predicted translation from a nucleic acid sequence. Accordingly, any amino acid sequence presented herein may contain errors due to errors in the nucleic acid sequence, as described in detail above. Furthermore, single nucleotide polymorphisms (SNPs) occur frequently in eukaryotic genomes—more than 1.4 million SNPs have already identified in the human genome, International Human Genome Sequencing Consortium, Nature 409:860-921 (2001)—and the sequence determined from one individual of a species may differ from other allelic forms present within the population. Small deletions and insertions can often be found that do not alter the function of the protein.

Accordingly, it is an aspect of the present invention to provide proteins not only identical in sequence to those described with particularity herein, but also to provide isolated proteins at least about 65% identical in sequence to those described with particularity herein, typically at least about 70%, 75%, 80%, 85%, or 90% identical in sequence to those described with particularity herein, usefully at least about 91%, 92%, 93%, 94%, or 95% identical in sequence to those described with particularity herein, usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention by way of random or directed mutagenesis.

For purposes herein, percent identity of two amino acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program BLAST 2 SEQUENCES, available online at http://www.ncbi.nlm.nih.gov/blast/bl2seq/bl2.html, To assess percent identity of amino acid sequences, the BLASTP module of BLAST 2 SEQUENCES is used with default values of (i) BLOSUM62 matrix, Henikoff et al., Proc. Natl. Acad. Sci USA 89(22):10915-9 (1992); (ii) open gap 11 and extension gap 1 penalties; and (iii) gap x_dropoff 50 expect 10 word size 3 filter, and both sequences are entered in their entireties.

As is well known, amino acid substitutions occur frequently among natural allelic variants, with conservative substitutions often occasioning only de minimis change in protein function.

Accordingly, it is an aspect of the present invention to provide proteins not only identical in sequence to those described with particularity herein, but also to provide isolated proteins having the sequence of LCP proteins, or portions thereof, with conservative amino acid substitutions. It is a further aspect to provide isolated proteins having the sequence of LCP proteins, and portions thereof, with moderately conservative amino acid substitutions. These conservatively-substituted and moderately conservatively-substituted variants can be naturally occurring or can result from human intervention.

Although there are a variety of metrics for calling conservative amino acid substitutions, based primarily on either observed changes among evolutionarily related proteins or on predicted chemical similarity, for purposes herein a conservative replacement is any change having a positive value in the PAM250 log-likelihood matrix reproduced herein below (see Gonnet et al., Science 256(5062):1443-5 (1992)):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 2 | −1 | 0 | 0 | 0 | 0 | 0 | 0 | −1 | −1 | −1 | 0 | −1 | −2 | 0 | 1 | 1 | −4 | −2 | 0 |
| R | −1 | 5 | 0 | 0 | −2 | 2 | 0 | −1 | 1 | −2 | −2 | 3 | −2 | −3 | −1 | 0 | 0 | −2 | −2 | −2 |
| N | 0 | 0 | 4 | 2 | −2 | 1 | 1 | 0 | 1 | −3 | −3 | 1 | −2 | −3 | −1 | 1 | 0 | −4 | −1 | −2 |
| D | 0 | 0 | 2 | 5 | −3 | 1 | 3 | 0 | 0 | −4 | −4 | 0 | −3 | −4 | −1 | 0 | 0 | −5 | −3 | −3 |
| C | 0 | −2 | −2 | −3 | 12 | −2 | −3 | −2 | −1 | −1 | −2 | −3 | −1 | −1 | −3 | 0 | 0 | −1 | 0 | 0 |
| Q | 0 | 2 | 1 | 1 | −2 | 3 | 2 | −1 | 1 | −2 | −2 | 2 | −1 | −3 | 0 | 0 | 0 | −3 | −2 | −2 |
| E | 0 | 0 | 1 | 3 | −3 | 2 | 4 | −1 | 0 | −3 | −3 | 1 | −2 | −4 | 0 | 0 | 0 | −4 | −3 | −2 |
| G | 0 | −1 | 0 | 0 | −2 | −1 | −1 | 7 | −1 | −4 | −4 | −1 | −4 | −5 | −2 | 0 | −1 | −4 | −4 | −3 |
| H | −1 | 1 | 1 | 0 | −1 | 1 | 0 | −1 | 6 | −2 | −2 | 1 | −1 | 0 | −1 | 0 | 0 | −1 | 2 | −2 |
| I | −1 | −2 | −3 | −4 | −1 | −2 | −3 | −4 | −2 | 4 | 3 | −2 | 2 | 1 | −3 | −2 | −1 | −2 | −1 | 3 |
| L | −1 | −2 | −3 | −4 | −2 | −2 | −3 | −4 | −2 | 3 | 4 | −2 | 3 | 2 | −2 | −2 | −1 | −1 | 0 | 2 |
| K | 0 | 3 | 1 | 0 | −3 | 2 | 1 | −1 | 1 | −2 | −2 | 3 | −1 | −3 | −1 | 0 | 0 | −4 | −2 | −2 |
| M | −1 | −2 | −2 | −3 | −1 | −1 | −2 | −4 | −1 | 2 | 3 | −1 | 4 | 2 | −2 | −1 | −1 | −1 | 0 | 2 |
| F | −2 | −3 | −3 | −4 | −1 | −3 | −4 | −5 | 0 | 1 | 2 | −3 | 2 | 7 | −4 | −3 | −2 | 4 | 5 | 0 |
| P | 0 | −1 | −1 | −1 | −3 | 0 | 0 | −2 | −1 | −3 | −2 | −1 | −2 | −4 | 8 | 0 | 0 | −5 | −3 | −2 |
| S | 1 | 0 | 1 | 0 | 0 | 0 | 0 | 0 | 0 | −2 | −2 | 0 | −1 | −3 | 0 | 2 | 2 | −3 | −2 | −1 |
| T | 1 | 0 | 0 | 0 | 0 | 0 | 0 | −1 | 0 | −1 | −1 | 0 | −1 | −2 | 0 | 2 | 2 | −4 | −2 | 0 |
| W | −4 | −2 | −4 | −5 | −1 | −3 | −4 | −4 | −1 | −2 | −1 | −4 | −1 | 4 | −5 | −3 | −4 | 14 | 4 | −3 |
| Y | −2 | −2 | −1 | −3 | 0 | −2 | −3 | −4 | 2 | −1 | 0 | −2 | 0 | 5 | −3 | −2 | −2 | 4 | 8 | −1 |
| V | 0 | −2 | −2 | −3 | 0 | −2 | −2 | −3 | −2 | 3 | 2 | −2 | 2 | 0 | −2 | −1 | 0 | −3 | −1 | 3 |

For purposes herein, a “moderately conservative” replacement is any change having a nonnegative value in the PAM250 log-likelihood matrix reproduced herein above.

As is also well known in the art, relatedness of proteins can also be characterized using a functional test, the ability of the encoding nucleic acids to base-pair to one another at defined hybridization stringencies.

It is, therefore, another aspect of the invention to provide isolated proteins not only identical in sequence to those described with particularity herein, but also to provide isolated proteins (“hybridization related proteins”) that are encoded by nucleic acids that hybridize under high stringency conditions (as defined herein above) to all or to a portion of various of the isolated nucleic acids of the present invention (“reference nucleic acids”). It is a further aspect of the invention to provide isolated proteins (“hybridization related proteins”) that are encoded by nucleic acids that hybridize under moderate stringency conditions (as defined herein above) to all or to a portion of various of the isolated nucleic acids of the present invention (“reference nucleic acids”).

The hybridization related proteins can be alternative isoforms, homologues, paralogues, and orthologues of the LCP protein of the present invention. Particularly useful orthologues are those from other primate species, such as chimpanzee, rhesus macaque monkey, baboon, orangutan, and gorilla, from rodents, such as rats, mice, guinea pigs; from lagomorphs, such as rabbits, and from domestic livestock, such as cow, pig, sheep, horse, and goat.

Relatedness of proteins can also be characterized using a second functional test, the ability of a first protein competitively to inhibit the binding of a second protein to an antibody.

It is, therefore, another aspect of the present invention to provide isolated proteins not only identical in sequence to those described with particularity herein, but also to provide isolated proteins (“cross-reactive proteins”) that competitively inhibit the binding of antibodies to all or to a portion of various of the isolated LCP proteins of the present invention (“reference proteins”). Such competitive inhibition can readily be determined using immunoassays well known in the art.

Among the proteins of the present invention that differ in amino acid sequence from those described with particularity herein—including those that have deletions and insertions causing up to 10% non-identity, those having conservative or moderately conservative substitutions, hybridization related proteins, and cross-reactive proteins—those that substantially retain one or more LCP activities are particularly useful. As described above, those activities include activities of CUB, LCCL or FA58C/DS domain.

Residues that are tolerant of change while retaining function can be identified by altering the protein at known residues using methods known in the art, such as alanine scanning mutagenesis, Cunningham et al., Science 244(4908):1081-5 (1989); transposon linker scanning mutagenesis, Chen et al., Gene 263(1-2):39-48 (2001); combinations of homolog- and alanine-scanning mutagenesis, Jin et al., J. Mol. Biol. 226(3):851-65 (1992); combinatorial alanine scanning, Weiss et al., Proc. Natl. Acad. Sci USA 97(16):8950-4 (2000), followed by functional assay. Transposon linker scanning kits are available commercially (New England Biolabs, Beverly, Mass., USA, catalog. no. E7-102S; EZ::TN™ In-Frame Linker Insertion Kit, catalogue no. EZI04KN, Epicentre Technologies Corporation, Madison, Wis., USA).

As further described below, the isolated proteins of the present invention can readily be used as specific immunogens to raise antibodies that specifically recognize LCP proteins, their isoforms, homologues, paralogues, and/or orthologues. The antibodies, in turn, can be used, inter alia, specifically to assay for the LCP proteins of the present invention—e.g. by ELISA for detection of protein fluid samples, such as serum, by immunohistochemistry or laser scanning cytometry, for detection of protein in tissue samples, or by flow cytometry, for detection of intracellular protein in cell suspensions—for specific antibody-mediated isolation and/or purification of LCP proteins, as for example by immunoprecipitation, and for use as specific agonists or antagonists of LCP action.

The isolated proteins of the present invention are also immediately available for use as specific standards in assays used to determine the concentration and/or amount specifically of the LCP proteins of the present invention. As is well known, ELISA kits for detection and quantitation of protein analytes typically include isolated and purified protein of known concentration for use as a measurement standard (e.g., the human interferon-γ OptEIA kit, catalog no. 555142, Pharmingen, San Diego, Calif., USA includes human recombinant gamma interferon, baculovirus produced).

The isolated proteins of the present invention are also immediately available for use as specific biomolecule capture probes for surface-enhanced laser desorption ionization (SELDI) detection of protein-protein interactions, WO 98/59362; WO 98/59360; WO 98/59361; and Merchant et al., Electrophoresis 21(6):1164-77 (2000), the disclosures of which are incorporated herein by reference in their entireties. Analogously, the isolated proteins of the present invention are also immediately available for use as specific biomolecule capture probes on BIACORE surface plasmon resonance probes. . See Weinberger et al., Pharmacogenomics 1(4):395-416 (2000); Malmqvist, Biochem. Soc. Trans. 27(2):335-40 (1999).

The isolated proteins of the present invention are also useful as a therapeutic supplement in patients having a specific deficiency in LCP production.

In another aspect, the invention also provides fragments of various of the proteins of the present invention. The protein fragments are useful, inter alia, as antigenic and immunogenic fragments of LCP.

By “fragments” of a protein is here intended isolated proteins (equally, polypeptides, peptides, oligopeptides), however obtained, that have an amino acid sequence identical to a portion of the reference amino acid sequence, which portion is at least 6 amino acids and less than the entirety of the reference nucleic acid. As so defined, “fragments” need not be obtained by physical fragmentation of the reference protein, although such provenance is not thereby precluded.

Fragments of at least 6 contiguous amino acids are useful in mapping B cell and T cell epitopes of the reference protein. See, e.g., Geysen et al., “Use of peptide synthesis to probe viral antigens for epitopes to a resolution of a single amino acid,” Proc. Natl. Acad. Sci. USA 81:3998-4002 (1984) and U.S. Pat. Nos. 4,708,871 and 5,595,915, the disclosures of which are incorporated herein by reference in their entireties. Because the fragment need not itself be immunogenic, part of an immunodominant epitope, nor even recognized by native antibody, to be useful in such epitope mapping, all fragments of at least 6 amino acids of the proteins of the present invention have utility in such a study.

Fragments of at least 8 contiguous amino acids, often at least 15 contiguous amino acids, have utility as immunogens for raising antibodies that recognize the proteins of the present invention. See, e.g., Lerner, “Tapping the immunological repertoire to produce antibodies of predetermined specificity,” Nature 299:592-596 (1982); Shinnick et al., “Synthetic peptide immunogens as vaccines,” Annu. Rev. Microbiol. 37:425-46 (1983); Sutcliffe et al., “Antibodies that react with predetermined sites on proteins,” Science 219:660-6 (1983), the disclosures of which are incorporated herein by reference in their entireties. As further described in the above-cited references, virtually all 8-mers, conjugated to a carrier, such as a protein, prove immunogenic—that is, prove capable of eliciting antibody for the conjugated peptide; accordingly, all fragments of at least 8 amino acids of the proteins of the present invention have utility as immunogens.

Fragments of at least 8, 9, 10 or 12 contiguous amino acids are also useful as competitive inhibitors of binding of the entire protein, or a portion thereof, to antibodies (as in epitope mapping), and to natural binding partners, such as subunits in a multimeric complex or to receptors or ligands of the subject protein; this competitive inhibition permits identification and separation of molecules that bind specifically to the protein of interest, U.S. Pat. Nos. 5,539,084 and 5,783,674, incorporated herein by reference in their entireties.

The protein, or protein fragment, of the present invention is thus at least 6 amino acids in length, typically at least 8, 9, 10 or 12 amino acids in length, and often at least 15 amino acids in length. Often, the protein or the present invention, or fragment thereof, is at least 20 amino acids in length, even 25 amino acids, 30 amino acids, 35 amino acids, or 50 amino acids or more in length. Of course, larger fragments having at least 75 amino acids, 100 amino acids, or even 150 amino acids are also useful, and at times preferred.

The present invention further provides fusions of each of the proteins and protein fragments of the present invention to heterologous polypeptides.

By fusion is here intended that the protein or protein fragment of the present invention is linearly contiguous to the heterologous polypeptide in a peptide-bonded polymer of amino acids or amino acid analogues; by “heterologous polypeptide” is here intended a polypeptide that does not naturally occur in contiguity with the protein or protein fragment of the present invention. As so defined, the fusion can consist entirely of a plurality of fragments of the LCP protein in altered arrangement; in such case, any of the LCP fragments can be considered heterologous to the other LCP fragments in the fusion protein. More typically, however, the heterologous polypeptide is not drawn from the LCP protein itself.

The fusion proteins of the present invention will include at least one fragment of the protein of the present invention, which fragment is at least 6, typically at least 8, often at least 15, and usefully at least 16, 17, 18, 19, or 20 amino acids long. The fragment of the protein of the present to be included in the fusion can usefully be at least 25 amino acids long, at least 50 amino acids long, and can be at least 75, 100, or even 150 amino acids long. Fusions that include the entirety of the proteins of the present invention have particular utility.

The heterologous polypeptide included within the fusion protein of the present invention is at least 6 amino acids in length, often at least 8 amino acids in length, and usefully at least 15, 20, and 25 amino acids in length. Fusions that include larger polypeptides, such as the IgG Fc region, and even entire proteins (such as GFP chromophore-containing proteins), have particular utility.

As described above in the description of vectors and expression vectors of the present invention, which discussion is incorporated herein by reference in its entirety, heterologous polypeptides to be included in the fusion proteins of the present invention can usefully include those designed to facilitate purification and/or visualization of recombinantly-expressed proteins. Although purification tags can also be incorporated into fusions that are chemically synthesized, chemical synthesis typically provides sufficient purity that further purification by HPLC suffices; however, visualization tags as above described retain their utility even when the protein is produced by chemical synthesis, and when so included render the fusion proteins of the present invention useful as directly detectable markers of LCP presence.

As also discussed above, heterologous polypeptides to be included in the fusion proteins of the present invention can usefully include those that facilitate secretion of recombinantly expressed proteins—into the periplasmic space or extracellular milieu for prokaryotic hosts, into the culture medium for eukaryotic cells—through incorporation of secretion signals and/or leader sequences.

Other useful protein fusions of the present invention include those that permit use of the protein of the present invention as bait in a yeast two-hybrid system. See Bartel et al. (eds.), The Yeast Two-Hybrid System, Oxford University Press (1997) (ISBN: 0195109384); Zhu et al., Yeast Hybrid Technologies, Eaton Publishing, (2000) (ISBN 1-881299-15-5); Fields et al., Trends Genet. 10(8):286-92 (1994); Mendelsohn et al., Curr. Opin. Biotechnol. 5(5):482-6 (1994); Luban et al., Curr. Opin. Biotechnol. 6(1):59-64 (1995); Allen et al., Trends Biochem. Sci. 20(12):511-6 (1995); Drees, Curr. Opin. Chem. Biol. 3(1):64-70 (1999); Topcu et al., Pharm. Res. 17(9):1049-55 (2000); Fashena et al., Gene 250(1-2):1-14 (2000), the disclosures of which are incorporated herein by reference in their entireties. Typically, such fusion is to either E. coli LexA or yeast GAL4 DNA binding domains. Related bait plasmids are available that express the bait fused to a nuclear localization signal.

Other useful protein fusions include those that permit display of the encoded protein on the surface of a phage or cell, fusions to intrinsically fluorescent proteins, such as green fluorescent protein (GFP), and fusions to the IgG Fc region, as described above, which discussion is incorporated here by reference in its entirety.

The proteins and protein fragments of the present invention can also usefully be fused to protein toxins, such as Pseudomonas exotoxin A, diphtheria toxin, shiga toxin A, anthrax toxin lethal factor, ricin, in order to effect ablation of cells that bind or take up the proteins of the present invention.

The isolated proteins, protein fragments, and protein fusions of the present invention can be composed of natural amino acids linked by native peptide bonds, or can contain any or all of nonnatural amino acid analogues, nonnative bonds, and post-synthetic (post translational) modifications, either throughout the length of the protein or localized to one or more portions thereof.

As is well known in the art, when the isolated protein is used, e.g., for epitope mapping, the range of such nonnatural analogues, nonnative inter-residue bonds, or post-synthesis modifications will be limited to those that permit binding of the peptide to antibodies. When used as an immunogen for the preparation of antibodies in a non-human host, such as a mouse, the range of such nonnatural analogues, nonnative inter-residue bonds, or post-synthesis modifications will be limited to those that do not interfere with the immunogenicity of the protein. When the isolated protein is used as a therapeutic agent, such as a vaccine or for replacement therapy, the range of such changes will be limited to those that do not confer toxicity upon the isolated protein.

Non-natural amino acids can be incorporated during solid phase chemical synthesis or by recombinant techniques, although the former is typically more common.

Solid phase chemical synthesis of peptides is well established in the art. Procedures are described, inter alia, in Chan et al. (eds.), Fmoc Solid Phase Peptide Synthesis: A Practical Approach (Practical Approach Series), Oxford Univ. Press (March 2000) (ISBN: 0199637245); Jones, Amino Acid and Peptide Synthesis (Oxford Chemistry Primers, No 7), Oxford Univ. Press (August 1992) (ISBN: 0198556683); and Bodanszky, Principles of Peptide Synthesis (Springer Laboratory), Springer Verlag (December 1993) (ISBN: 0387564314), the disclosures of which are incorporated herein by reference in their entireties.

For example, D-enantiomers of natural amino acids can readily be incorporated during chemical peptide synthesis: peptides assembled from D-amino acids are more resistant to proteolytic attack; incorporation of D-enantiomers can also be used to confer specific three dimensional conformations on the peptide. Other amino acid analogues commonly added during chemical synthesis include ornithine, norleucine, phosphorylated amino acids (typically phosphoserine, phosphothreonine, phosphotyrosine), L-malonyltyrosine, a non-hydrolyzable analog of phosphotyrosine (Kole et al., Biochem. Biophys. Res. Com. 209:817-821 (1995)), and various halogenated phenylalanine derivatives.

Amino acid analogues having detectable labels are also usefully incorporated during synthesis to provide a labeled polypeptide.

Biotin, for example (indirectly detectable through interaction with avidin, streptavidin, neutravidin, captavidin, or anti-biotin antibody), can be added using biotinoyl-(9-fluorenylmethoxycarbonyl)-L-lysine (FMOC biocytin) (Molecular Probes, Eugene, Oreg., USA). (Biotin can also be added enzymatically by incorporation into a fusion protein of a E. coli BirA substrate peptide.).

The FMOC and tBOC derivatives of dabcyl-L-lysine (Molecular Probes, Inc., Eugene, Oreg., USA) can be used to incorporate the dabcyl chromophore at selected sites in the peptide sequence during synthesis. The aminonaphthalene derivative EDANS, the most common fluorophore for pairing with the dabcyl quencher in fluorescence resonance energy transfer (FRET) systems, can be introduced during automated synthesis of peptides by using EDANS-FMOC-L-glutamic acid or the corresponding TBOC derivative (both from Molecular Probes, Inc., Eugene, Oreg., USA). Tetramethylrhodamine fluorophores can be incorporated during automated FMOC synthesis of peptides using (FMOC)-TMR-L-lysine (Molecular Probes, Inc. Eugene, Oreg., USA).

Other useful amino acid analogues that can be incorporated during chemical synthesis include aspartic acid, glutamic acid, lysine, and tyrosine analogues having allyl side-chain protection (Applied Biosystems, Inc., Foster City, Calif., USA); the allyl side chain permits synthesis of cyclic, branched-chain, sulfonated, glycosylated, and phosphorylated peptides.

A large number of other FMOC-protected non-natural amino acid analogues capable of incorporation during chemical synthesis are available commercially, including, e.g., Fmoc-2-aminobicyclo[2.2.1]heptane-2-carboxylic acid, Fmoc-3-endo-aminobicyclo[2.2.1]heptane-2-endo-carboxylic acid, Fmoc-3-exo-aminobicyclo[2.2.1]heptane-2-exo-carboxylic acid, Fmoc-3-endo-amino-bicyclo[2.2.1]hept-5-ene-2-endo-carboxylic acid, Fmoc-3-exo-amino-bicyclo[2.2.1]hept-5-ene-2-exo-carboxylic acid, Fmoc-cis-2-amino-1-cyclohexanecarboxylic acid, Fmoc-trans-2-amino-1-cyclohexanecarboxylic acid, Fmoc-1-amino-1-cyclopentanecarboxylic acid, Fmoc-cis-2-amino-1-cyclopentanecarboxylic acid, Fmoc-1-amino-1-cyclopropanecarboxylic acid, Fmoc-D-2-amino-4-(ethylthio)butyric acid, Fmoc-L-2-amino-4-(ethylthio)butyric acid, Fmoc-L-buthionine, Fmoc-S-methyl-L-Cysteine, Fmoc-2-aminobenzoic acid (anthranillic acid), Fmoc-3-aminobenzoic acid, Fmoc-4-aminobenzoic acid, Fmoc-2-aminobenzophenone-2′-carboxylic acid, Fmoc-N-(4-aminobenzoyl)-b-alanine, Fmoc-2-amino-4,5-dimethoxybenzoic acid, Fmoc-4-aminohippuric acid, Fmoc-2-amino-3-hydroxybenzoic acid, Fmoc-2-amino-5-hydroxybenzoic acid, Fmoc-3-amino-4-hydroxybenzoic acid, Fmoc-4-amino-3-hydroxybenzoic acid, Fmoc-4-amino-2-hydroxybenzoic acid, Fmoc-5-amino-2-hydroxybenzoic acid, Fmoc-2-amino-3-methoxybenzoic acid, Fmoc-4-amino-3-methoxybenzoic acid, Fmoc-2-amino-3-methylbenzoic acid, Fmoc-2-amino-5-methylbenzoic acid, Fmoc-2-amino-6-methylbenzoic acid, Fmoc-3-amino-2-methylbenzoic acid, Fmoc-3-amino-4-methylbenzoic acid, Fmoc-4-amino-3-methylbenzoic acid, Fmoc-3-amino-2-naphtoic acid, Fmoc-D,L-3-amino-3-phenylpropionic acid, Fmoc-L-Methyldopa, Fmoc-2-amino-4,6-dimethyl-3-pyridinecarboxylic acid, Fmoc-D,L-?-amino-2-thiophenacetic acid, Fmoc-4-(carboxymethyl)piperazine, Fmoc-4-carboxypiperazine, Fmoc-4-(carboxymethyl)homopiperazine, Fmoc-4-phenyl-4-piperidinecarboxylic acid, Fmoc-L-1,2,3,4-tetrahydronorharman-3-carboxylic acid, Fmoc-L-thiazolidine-4-carboxylic acid, all available from The Peptide Laboratory (Richmond, Calif., USA).

Non-natural residues can also be added biosynthetically by engineering a suppressor tRNA, typically one that recognizes the UAG stop codon, by chemical aminoacylation with the desired unnatural amino acid and. Conventional site-directed mutagenesis is used to introduce the chosen stop codon UAG at the site of interest in the protein gene. When the acylated suppressor tRNA and the mutant gene are combined in an in vitro transcription/translation system, the unnatural amino acid is incorporated in response to the UAG codon to give a protein containing that amino acid at the specified position. Liu et al., Proc. Natl Acad. Sci. USA 96(9):4780-5 (1999); Wang et al., Science 292(5516):498-500 (2001).

The isolated proteins, protein fragments and fusion proteins of the present invention can also include nonnative inter-residue bonds, including bonds that lead to circular and branched forms.

The isolated proteins and protein fragments of the present invention can also include post-translational and post-synthetic modifications, either throughout the length of the protein or localized to one or more portions thereof.

For example, when produced by recombinant expression in eukaryotic cells, the isolated proteins, fragments, and fusion proteins of the present invention will typically include N-linked and/or O-linked glycosylation, the pattern of which will reflect both the availability of glycosylation sites on the protein sequence and the identity of the host cell. Further modification of glycosylation pattern can be performed enzymatically.

As another example, recombinant polypeptides of the invention may also include an initial modified methionine residue, in some cases resulting from host-mediated processes.

When the proteins, protein fragments, and protein fusions of the present invention are produced by chemical synthesis, post-synthetic modification can be performed before deprotection and cleavage from the resin or after deprotection and cleavage. Modification before deprotection and cleavage of the synthesized protein often allows greater control, e.g. by allowing targeting of the modifying moiety to the N-terminus of a resin-bound synthetic peptide.

Useful post-synthetic (and post-translational) modifications include conjugation to detectable labels, such as fluorophores.

A wide variety of amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other.

Kits are available commercially that permit conjugation of proteins to a variety of amine-reactive or thiol-reactive fluorophores: Molecular Probes, Inc. (Eugene, Oreg., USA), e.g., offers kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, and Texas Red-X.

A wide variety of other amine-reactive and thiol-reactive fluorophores are available commercially (Molecular Probes, Inc., Eugene, Oreg., USA), including Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA).

The polypeptides of the present invention can also be conjugated to fluorophores, other proteins, and other macromolecules, using bifunctional linking reagents.

Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA); common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available Pierce, Rockford, Ill., USA).

The proteins, protein fragments, and protein fusions of the present invention can be conjugated, using such cross-linking reagents, to fluorophores that are not amine- or thiol-reactive.

Other labels that usefully can be conjugated to the proteins, protein fragments, and fusion proteins of the present invention include radioactive labels, echosonographic contrast reagents, and MRI contrast agents.

The proteins, protein fragments, and protein fusions of the present invention can also usefully be conjugated using cross-linking agents to carrier proteins, such as KLH, bovine thyroglobulin, and even bovine serum albumin (BSA), to increase immunogenicity for raising anti-LCP antibodies.

The proteins, protein fragments, and protein fusions of the present invention can also usefully be conjugated to polyethylene glycol (PEG); PEGylation increases the serum half life of proteins administered intravenously for replacement therapy. Delgado et al., Crit. Rev. Ther. Drug Carrier Syst. 9(3-4):249-304 (1992); Scott et al., Curr. Pharm. Des. 4(6):423-38 (1998); DeSantis et al., Curr. Opin. Biotechnol. 10(4):324-30 (1999), incorporated herein by reference in their entireties. PEG monomers can be attached to the protein directly or through a linker, with PEGylation using PEG monomers activated with tresyl chloride (2,2,2-trifluoroethanesulphonyl chloride) permitting direct attachment under mild conditions.

The isolated proteins of the present invention, including fusions thereof, can be produced by recombinant expression, typically using the expression vectors of the present invention as above-described or, if fewer than about 100 amino acids, by chemical synthesis (typically, solid phase synthesis), and, on occasion, by in vitro translation.

Production of the isolated proteins of the present invention can optionally be followed by purification. Purification of recombinantly expressed proteins is now well within the skill in the art. See, e.g., Thorner et al. (eds.), Applications of Chimeric Genes and Hybrid Proteins, Part A: Gene Expression and Protein Purification (Methods in Enzymology, Volume 326), Academic Press (2000), (ISBN: 0121822273); Harbin (ed.), Cloning, Gene Expression and Protein Purification: Experimental Procedures and Process Rationale, Oxford Univ. Press (2001) (ISBN: 0195132947); Marshak et al., Strategies for Protein Purification and Characterization: A Laboratory Course Manual, Cold Spring Harbor Laboratory Press (1996) (ISBN: 0-87969-385-1); and Roe (ed.), Protein Purification Applications, Oxford University Press (2001), the disclosures of which are incorporated herein by reference in their entireties, and thus need not be detailed here.

Briefly, however, if purification tags have been fused through use of an expression vector that appends such tag, purification can be effected, at least in part, by means appropriate to the tag, such as use of immobilized metal affinity chromatography for polyhistidine tags. Other techniques common in the art include ammonium sulfate fractionation, immunoprecipitation, fast protein liquid chromatography (FPLC), high performance liquid chromatography (HPLC), and preparative gel electrophoresis.

Purification of chemically-synthesized peptides can readily be effected, e.g., by HPLC.

Accordingly, it is an aspect of the present invention to provide the isolated proteins of the present invention in pure or substantially pure form.

A purified protein of the present invention is an isolated protein, as above described, that is present at a concentration of at least 95%, as measured on a weight basis (w/w) with respect to total protein in a composition. Such purities can often be obtained during chemical synthesis without further purification, as, e.g., by HPLC. Purified proteins of the present invention can be present at a concentration (measured on a weight basis with respect to total protein in a composition) of 96%, 97%, 98%, and even 99%. The proteins of the present invention can even be present at levels of 99.5%, 99.6%, and even 99.7%, 99.8%, or even 99.9% following purification, as by HPLC.

Although high levels of purity are particularly useful when the isolated proteins of the present invention are used as therapeutic agents—such as vaccines, or for replacement therapy—the isolated proteins of the present invention are also useful at lower purity. For example, partially purified proteins of the present invention can be used as immunogens to raise antibodies in laboratory animals.

Thus, in another aspect, the present invention provides the isolated proteins of the present invention in substantially purified form. A “substantially purified protein” of the present invention is an isolated protein, as above described, present at a concentration of at least 70%, measured on a weight basis with respect to total protein in a composition. Usefully, the substantially purified protein is present at a concentration, measured on a weight basis with respect to total protein in a composition, of at least 75%, 80%, or even at least 85%, 90%, 91%, 92%, 93%, 94%, 94.5% or even at least 94.9%.

In preferred embodiments, the purified and substantially purified proteins of the present invention are in compositions that lack detectable ampholytes, acrylamide monomers, bis-acrylamide monomers, and polyacrylamide.

The proteins, fragments, and fusions of the present invention can usefully be attached to a substrate. The substrate can porous or solid, planar or non-planar; the bond can be covalent or noncovalent.

For example, the proteins, fragments, and fusions of the present invention can usefully be bound to a porous substrate, commonly a membrane, typically comprising nitrocellulose, polyvinylidene fluoride (PVDF), or cationically derivatized, hydrophilic PVDF; so bound, the proteins, fragments, and fusions of the present invention can be used to detect and quantify antibodies, e.g. in serum, that bind specifically to the immobilized protein of the present invention.

As another example, the proteins, fragments, and fusions of the present invention can usefully be bound to a substantially nonporous substrate, such as plastic, to detect and quantify antibodies, e.g. in serum, that bind specifically to the immobilized protein of the present invention. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof; when the assay is performed in standard microtiter dish, the plastic is typically polystyrene.

The proteins, fragments, and fusions of the present invention can also be attached to a substrate suitable for use as a surface enhanced laser desorption ionization source; so attached, the protein, fragment, or fusion of the present invention is useful for binding and then detecting secondary proteins that bind with sufficient affinity or avidity to the surface-bound protein to indicate biologic interaction therebetween. The proteins, fragments, and fusions of the present invention can also be attached to a substrate suitable for use in surface plasmon resonance detection; so attached, the protein, fragment, or fusion of the present invention is useful for binding and then detecting secondary proteins that bind with sufficient affinity or avidity to the surface-bound protein to indicate biological interaction therebetween.

LCP Proteins

In a first series of protein embodiments, the invention provides isolated LCP polypeptides having amino acid sequences in SEQ ID NO: 3 or 1114, which are full length LCP1 and LCP2 proteins. When used as immunogens, the full length proteins of the present invention can be used, inter alia, to elicit antibodies that bind to a variety of epitopes of the LCP proteins.

The invention further provides fragments of the above-described polypeptides, particularly fragments having at least 6 amino acids, typically at least 8 amino acids, often at least 15 amino acids, and even the entirety of the sequence given in SEQ ID NOs: 3 or 1114.

The invention further provides fragments of at least 6 amino acids, typically at least 8 amino acids, often at least 15 amino acids, and even the entirety of the sequence given in SEQ ID NO: 5.

The invention also provides fragments of at least 6 amino acids, typically at least 8 amino acids, often at least 15 amino acids, and even the entirety of the sequence given in SEQ ID NO: 9.

The invention also provides fragments of at least 6 amino acids, typically at least 8 amino acids, often at least 15 amino acids, and even the entirety of the sequence given in SEQ ID NO: 1116.

As described above, the invention further provides proteins that differ in sequence from those described with particularity in the above-referenced SEQ ID NOs., whether by way of insertion or deletion, by way of conservative or moderately conservative substitutions, as hybridization related proteins, or as cross-hybridizing proteins, with those that substantially retain an LCP activity particularly useful.

The invention further provides fusions of the proteins and protein fragments herein described to heterologous polypeptides.

Antibodies and Antibody-Producing Cells

In another aspect, the invention provides antibodies, including fragments and derivatives thereof, that bind specifically to LCP proteins and protein fragments of the present invention or to one or more of the proteins and protein fragments encoded by the isolated LCP nucleic acids of the present invention. The antibodies of the present invention can be specific for all of linear epitopes, discontinuous epitopes, or conformational epitopes of such proteins or protein fragments, either as present on the protein in its native conformation or, in some cases, as present on the proteins as denatured, as, e.g., by solubilization in SDS.

In other embodiments, the invention provides antibodies, including fragments and derivatives thereof, the binding of which can be competitively inhibited by one or more of the LCP proteins and protein fragments of the present invention, or by one or more of the proteins and protein fragments encoded by the isolated LCP nucleic acids of the present invention.

As used herein, the term “antibody” refers to a polypeptide, at least a portion of which is encoded by at least one immunoglobulin gene, which can bind specifically to a first molecular species, and to fragments or derivatives thereof that remain capable of such specific binding.

By “bind specifically” and “specific binding” is here intended the ability of the antibody to bind to a first molecular species in preference to binding to other molecular species with which the antibody and first molecular species are admixed. An antibody is said specifically to “recognize” a first molecular species when it can bind specifically to that first molecular species.

As is well known in the art, the degree to which an antibody can discriminate as among molecular species in a mixture will depend, in part, upon the conformational relatedness of the species in the mixture; typically, the antibodies of the present invention will discriminate over adventitious binding to non-LCP proteins by at least two-fold, more typically by at least 5-fold, typically by more than 10-fold, 25-fold, 50-fold, 75-fold, and often by more than 100-fold, and on occasion by more than 500-fold or 1000-fold. When used to detect the proteins or protein fragments of the present invention, the antibody of the present invention is sufficiently specific when it can be used to determine the presence of the protein of the present invention in samples derived from human adrenal, adult liver, bone marrow, brain, fetal liver, heart, kidney, lung, placenta, skeletal muscle, colon and prostate, as well as a cell line, hela.

Typically, the affinity or avidity of an antibody (or antibody multimer, as in the case of an IgM pentamer) of the present invention for a protein or protein fragment of the present invention will be at least about 1×10⁻⁶ molar (M), typically at least about 5×10⁻⁷ M, usefully at least about 1×10⁻⁷ M, with affinities and avidities of at least 1×10⁻⁸ M, 5×10⁻⁹ M, and 1×10⁻¹⁰ M proving especially useful.

The antibodies of the present invention can be naturally-occurring forms, such as IgG, IgM, IgD, IgE, and IgA, from any mammalian species.

Human antibodies can, but will infrequently, be drawn directly from human donors or human cells. In such case, antibodies to the proteins of the present invention will typically have resulted from fortuitous immunization, such as autoimmune immunization, with the protein or protein fragments of the present invention. Such antibodies will typically, but will not invariably, be polyclonal.

Human antibodies are more frequently obtained using transgenic animals that express human immunoglobulin genes, which transgenic animals can be affirmatively immunized with the protein immunogen of the present invention. Human Ig-transgenic mice capable of producing human antibodies and methods of producing human antibodies therefrom upon specific immunization are described, inter alia, in U.S. Pat. Nos. 6,162,963; 6,150,584; 6,114,598; 6,075,181; 5,939,598; 5,877,397; 5,874,299; 5,814,318; 5,789,650; 5,770,429; 5,661,016; 5,633,425; 5,625,126; 5,569,825; 5,545,807; 5,545,806, and 5,591,669, the disclosures of which are incorporated herein by reference in their entireties. Such antibodies are typically monoclonal, and are typically produced using techniques developed for production of murine antibodies.

Human antibodies are particularly useful, and often preferred, when the antibodies of the present invention are to be administered to human beings as in vivo diagnostic or therapeutic agents, since recipient immune response to the administered antibody will often be substantially less than that occasioned by administration of an antibody derived from another species, such as mouse.

IgG, IgM, IgD, IgE and IgA antibodies of the present invention are also usefully obtained from other mammalian species, including rodents—typically mouse, but also rat, guinea pig, and hamster—lagomorphs, typically rabbits, and also larger mammals, such as sheep, goats, cows, and horses. In such cases, as with the transgenic human-antibody-producing non-human mammals, fortuitous immunization is not required, and the non-human mammal is typically affirmatively immunized, according to standard immunization protocols, with the protein or protein fragment of the present invention.

As discussed above, virtually all fragments of 8 or more contiguous amino acids of the proteins of the present invention can be used effectively as immunogens when conjugated to a carrier, typically a protein such as bovine thyroglobulin, keyhole limpet hemocyanin, or bovine serum albumin, conveniently using a bifunctional linker such as those described elsewhere above, which discussion is incorporated by reference here.

Immunogenicity can also be conferred by fusion of the proteins and protein fragments of the present invention to other moieties.

For example, peptides of the present invention can be produced by solid phase synthesis on a branched polylysine core matrix; these multiple antigenic peptides (MAPs) provide high purity, increased avidity, accurate chemical definition and improved safety in vaccine development. Tam et al., Proc. Natl. Acad. Sci. USA 85:5409-5413 (1988); Posnett et al., J. Biol. Chem. 263, 1719-1725 (1988).

Protocols for immunizing non-human mammals are well-established in the art, Harlow et al. (eds.), Antibodies: A Laboratory Manual, Cold Spring Harbor Laboratory (1998) (ISBN: 0879693142); Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907), the disclosures of which are incorporated herein by reference, and often include multiple immunizations, either with or without adjuvants such as Freund's complete adjuvant and Freund's incomplete adjuvant.

Antibodies from nonhuman mammals can be polyclonal or monoclonal, with polyclonal antibodies having certain advantages in immunohistochemical detection of the proteins of the present invention and monoclonal antibodies having advantages in identifying and distinguishing particular epitopes of the proteins of the present invention.

Following immunization, the antibodies of the present invention can be produced using any art-accepted technique. Such techniques are well known in the art, Coligan et al. (eds.), Current Protocols in Immunology, John Wiley & Sons, Inc. (2001) (ISBN: 0-471-52276-7); Zola, Monoclonal Antibodies Preparation and Use of Monoclonal Antibodies and Engineered Antibody Derivatives (Basics: From Background to Bench), Springer Verlag (2000) (ISBN: 0387915907); Howard et al. (eds.), Basic Methods in Antibody Production and Characterization, CRC Press (2000) (ISBN: 0849394457); Harlow et al. (eds.), Antibodies: A Laboratory Manual, Cold Spring Harbor Laboratory (1998) (ISBN: 0879693142); Davis (ed.), Monoclonal Antibody Protocols, Vol. 45, Humana Press (1995) (ISBN: 0896033082); Delves (ed.), Antibody Production: Essential Techniques, John Wiley & Son Ltd (1997) (ISBN: 0471970107); Kenney, Antibody Solution: An Antibody Methods Manual, Chapman & Hall (1997) (ISBN: 0412141914), incorporated herein by reference in their entireties, and thus need not be detailed here.

Briefly, however, such techniques include, inter alia, production of monoclonal antibodies by hybridomas and expression of antibodies or fragments or derivatives thereof from host cells engineered to express immunoglobulin genes or fragments thereof. These two methods of production are not mutually exclusive: genes encoding antibodies specific for the proteins or protein fragments of the present invention can be cloned from hybridomas and thereafter expressed in other host cells. Nor need the two necessarily be performed together: e.g., genes encoding antibodies specific for the proteins and protein fragments of the present invention can be cloned directly from B cells known to be specific for the desired protein, as further described in U.S. Pat. No. 5,627,052, the disclosure of which is incorporated herein by reference in its entirety, or from antibody-displaying phage.

Recombinant expression in host cells is particularly useful when fragments or derivatives of the antibodies of the present invention are desired.

Host cells for recombinant antibody production—either whole antibodies, antibody fragments, or antibody derivatives—can be prokaryotic or eukaryotic.

Prokaryotic hosts are particularly useful for producing phage displayed antibodies of the present invention.

The technology of phage-displayed antibodies, in which antibody variable region fragments are fused, for example, to the gene III protein (pIII) or gene VIII protein (pVIII) for display on the surface of filamentous phage, such as M13, is by now well-established, Sidhu, Curr. Opin. Biotechnol. 11(6):610-6 (2000); Griffiths et al., Curr. Opin. Biotechnol. 9(1):102-8 (1998); Hoogenboom et al., Immunotechnology, 4(1):1-20 (1998); Rader et al., Current Opinion in Biotechnology 8:503-508 (1997); Aujame et al., Human Antibodies 8:155-168 (1997); Hoogenboom, Trends in Biotechnol. 15:62-70 (1997); de Kruif et al., 17:453-455 (1996); Barbas et al., Trends in Biotechnol. 14:230-234 (1996); Winter et al., Ann. Rev. Immunol. 433-455 (1994), and techniques and protocols required to generate, propagate, screen (pan), and use the antibody fragments from such libraries have recently been compiled, Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, Academic Press, Inc. (1996); Abelson et al. (eds.), Combinatorial Chemistry, Methods in Enzymology vol. 267, Academic Press (May 1996), the disclosures of which are incorporated herein by reference in their entireties.

Typically, phage-displayed antibody fragments are scFv fragments or Fab fragments; when desired, full length antibodies can be produced by cloning the variable regions from the displaying phage into a complete antibody and expressing the full length antibody in a further prokaryotic or a eukaryotic host cell.

Eukaryotic cells are also useful for expression of the antibodies, antibody fragments, and antibody derivatives of the present invention.

For example, antibody fragments of the present invention can be produced in Pichia pastoris, Takahashi et al., Biosci. Biotechnol. Biochem. 64(10):2138-44 (2000); Freyre et al., J. Biotechnol. 76(2-3):157-63 (2000); Fischer et al., Biotechnol. Appl. Biochem. 30 (Pt 2):117-20 (1999); Pennell et al., Res. Immunol. 149(6):599-603 (1998); Eldin et al., J. Immunol. Methods. 201(l):67-75 (1997); and in Saccharomyces cerevisiae, Frenken et al., Res. Immunol. 149(6):589-99 (1998); Shusta et al., Nature Biotechnol. 16(8):773-7 (1998), the disclosures of which are incorporated herein by reference in their entireties.

Antibodies, including antibody fragments and derivatives, of the present invention can also be produced in insect cells, Li et al., Protein Expr. Purif. 21(1):121-8 (2001); Ailor et al., Biotechnol. Bioeng. 58(2-3):196-203 (1998); Hsu et al., Biotechnol. Prog. 13(1):96-104 (1997); Edelman et al., Immunology 91(1):13-9 (1997); and Nesbit et al., J. Immunol. Methods. 151(1-2):201-8 (1992), the disclosures of which are incorporated herein by reference in their entireties.

Antibodies and fragments and derivatives thereof of the present invention can also be produced in plant cells, Giddings et al., Nature Biotechnol. 18(11):1151-5 (2000); Gavilondo et al., Biotechniques 29(1):128-38 (2000); Fischer et al., J. Biol. Regul. Homeost. Agents 14(2):83-92 (2000); Fischer et al., Biotechnol. Appl. Biochem. 30 (Pt 2):113-6 (1999); Fischer et al., Biol. Chem. 380(7-8):825-39 (1999); Russell, Curr. Top. Microbiol. Immunol. 240:119-38 (1999); and Ma et al., Plant Physiol. 109(2):341-6 (1995), the disclosures of which are incorporated herein by reference in their entireties.

Mammalian cells useful for recombinant expression of antibodies, antibody fragments, and antibody derivatives of the present invention include CHO cells, COS cells, 293 cells, and myeloma cells.

Verma et al., J. Immunol. Methods 216(1-2):165-81 (1998), review and compare bacterial, yeast, insect and mammalian expression systems for expression of antibodies.

Antibodies of the present invention can also be prepared by cell free translation, as further described in Merk et al., J. Biochem. (Tokyo). 125(2):328-33 (1999) and Ryabova et al., Nature Biotechnol. 15(l):79-84 (1997), and in the milk of transgenic animals, as further described in Pollock et al., J. Immunol. Methods 231(1-2):147-57 (1999), the disclosures of which are incorporated herein by reference in their entireties.

The invention further provides antibody fragments that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Among such useful fragments are Fab, Fab′, Fv, F(ab)′₂, and single chain Fv (scFv) fragments. Other useful fragments are described in Hudson, Curr. Opin. Biotechnol. 9(4):395-402 (1998).

It is also an aspect of the present invention to provide antibody derivatives that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Among such useful derivatives are chimeric, primatized, and humanized antibodies; such derivatives are less immunogenic in human beings, and thus more suitable for in vivo administration, than are unmodified antibodies from non-human mammalian species.

Chimeric antibodies typically include heavy and/or light chain variable regions (including both CDR and framework residues) of immunoglobulins of one species, typically mouse, fused to constant regions of another species, typically human. See, e.g., U.S. Pat. No. 5,807,715; Morrison et al., Proc. Natl. Acad. Sci USA.81(21):6851-5 (1984); Sharon et al., Nature 309(5966):364-7 (1984); Takeda et al., Nature 314(6010):452-4 (1985), the disclosures of which are incorporated herein by reference in their entireties. Primatized and humanized antibodies typically include heavy and/or light chain CDRs from a murine antibody grafted into a non-human primate or human antibody V region framework, usually further comprising a human constant region, Riechmann et al., Nature 332(6162):323-7 (1988); Co et al., Nature 351(6326):501-2 (1991); U.S. Pat. Nos. 6,054,297; 5,821,337; 5,770,196; 5,766,886; 5,821,123; 5,869,619; 6,180,377; 6,013,256; 5,693,761; and 6,180,370, the disclosures of which are incorporated herein by reference in their entireties.

Other useful antibody derivatives of the invention include heteromeric antibody complexes and antibody fusions, such as diabodies (bispecific antibodies), single-chain diabodies, and intrabodies.

The antibodies of the present invention, including fragments and derivatives thereof, can usefully be labeled. It is, therefore, another aspect of the present invention to provide labeled antibodies that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

The choice of label depends, in part, upon the desired use.

For example, when the antibodies of the present invention are used for immunohistochemical staining of tissue samples, the label can usefully be an enzyme that catalyzes production and local deposition of a detectable product.

Enzymes typically conjugated to antibodies to permit their immunohistochemical visualization are well known, and include alkaline phosphatase, β-galactosidase, glucose oxidase, horseradish peroxidase (HRP), and urease. Typical substrates for production and deposition of visually detectable products include o-nitrophenyl-beta-D-galactopyranoside (ONPG); o-phenylenediamine dihydrochloride (OPD); p-nitrophenyl phosphate (PNPP); p-nitrophenyl-beta-D-galactopryanoside (PNPG); 3′,3′-diaminobenzidine (DAB); 3-amino-9-ethylcarbazole (AEC); 4-chloro-1-naphthol (CN); 5-bromo-4-chloro-3-indolyl-phosphate (BCIP); ABTS®; BluoGal; iodonitrotetrazolium (INT); nitroblue tetrazolium chloride (NBT); phenazine methosulfate (PMS); phenolphthalein monophosphate (PMP); tetramethyl benzidine (TMB); tetranitroblue tetrazolium (TNBT); X-Gal; X-Gluc; and X-Glucoside.

Other substrates can be used to produce products for local deposition that are luminescent. For example, in the presence of hydrogen peroxide (H₂O₂), horseradish peroxidase (HRP) can catalyze the oxidation of cyclic diacylhydrazides, such as luminol. Immediately following the oxidation, the luminol is in an excited state (intermediate reaction product), which decays to the ground state by emitting light. Strong enhancement of the light emission is produced by enhancers, such as phenolic compounds. Advantages include high sensitivity, high resolution, and rapid detection without radioactivity and requiring only small amounts of antibody. See, e.g., Thorpe et al., Methods Enzymol. 133:331-53 (1986); Kricka et al., J. Inmunoassay 17(1):67-83 (1996); and Lundqvist et al., J. Biolumin. Chemilumin. 10(6):353-9 (1995), the disclosures of which are incorporated herein by reference in their entireties. Kits for such enhanced chemiluminescent detection (ECL) are available commercially.

The antibodies can also be labeled using colloidal gold.

As another example, when the antibodies of the present invention are used, e.g., for flow cytometric detection, for scanning laser cytometric detection, or for fluorescent immunoassay, they can usefully be labeled with fluorophores.

There are a wide variety of fluorophore labels that can usefully be attached to the antibodies of the present invention.

For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCP-Cy5.5, PE-Cy5, PE-Cy5.5, PE-Cy7, PE-Texas Red, and APC-Cy7.

Other fluorophores include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, Cy7, all of which are also useful for fluorescently labeling the antibodies of the present invention.

For secondary detection using labeled avidin, streptavidin, captavidin or neutravidin, the antibodies of the present invention can usefully be labeled with biotin.

When the antibodies of the present invention are used, e.g., for western blotting applications, they can usefully be labeled with radioisotopes, such as ³³P, ³²P, ³⁵S, ³H, and I²⁵¹.

As another example, when the antibodies of the present invention are used for radioimmunotherapy, the label can usefully be ²²⁸Th, ²²⁷Ac, ²²⁵Ac, ²²³Ra, ²¹³Bi, ²¹²Pb, ²¹²Bi, ²At, ²⁰³Pb, ¹⁹⁴Os, ¹⁸⁸Re, ¹⁸⁶Re, ¹⁵³Sm, ¹⁴⁹Tb, ¹³³I, ¹²⁵I, ¹¹¹In, ¹⁰⁵ Rh, ^{99m}Tc, ⁹⁷R, ⁹⁰Y, ⁹⁰Sr, ⁸⁸Y, ⁷²Se, ⁶⁷Cu, or 47Sc.

As another example, when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2):529-38 (1998), or by radioisotopic labeling.

As would be understood, use of the labels described above is not restricted to the application as for which they were mentioned.

The antibodies of the present invention, including fragments and derivatives thereof, can also be conjugated to toxins, in order to target the toxin's ablative action to cells that display and/or express the proteins of the present invention. Commonly, the antibody in such immunotoxins is conjugated to Pseudomonas exotoxin A, diphtheria toxin, shiga toxin A, anthrax toxin lethal factor, or ricin. See Hall (ed.), Immunotoxin Methods and Protocols (Methods in Molecular Biology, Vol 166), Humana Press (2000) (ISBN:0896037754); and Frankel et al. (eds.), Clinical Applications of Immunotoxins, Springer-Verlag New York, Incorporated (1998) (ISBN:3540640975), the disclosures of which are incorporated herein by reference in their entireties, for review.

The antibodies of the present invention can usefully be attached to a substrate, and it is, therefore, another aspect of the invention to provide antibodies that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention, attached to a substrate.

Substrates can be porous or nonporous, planar or nonplanar.

For example, the antibodies of the present invention can usefully be conjugated to filtration media, such as NHS-activated Sepharose or CNBr-activated Sepharose for purposes of immunoaffinity chromatography.

For example, the antibodies of the present invention can usefully be attached to paramagnetic microspheres, typically by biotin-streptavidin interaction, which microsphere can then be used for isolation of cells that express or display the proteins of the present invention. As another example, the antibodies of the present invention can usefully be attached to the surface of a microtiter plate for ELISA.

As noted above, the antibodies of the present invention can be produced in prokaryotic and eukaryotic cells. It is, therefore, another aspect of the present invention to provide cells that express the antibodies of the present invention, including hybridoma cells, B cells, plasma cells, and host cells recombinantly modified to express the antibodies of the present invention.

In yet a further aspect, the present invention provides aptamers evolved to bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

LCP Antibodies

In a first series of antibody embodiments, the invention provides antibodies, both polyclonal and monoclonal, and fragments and derivatives thereof, that bind specifically to a polypeptide having an amino acid sequence in SEQ ID NOs: 3 or 1114, which are full length LCP1 and LCP2 proteins.

Such antibodies are useful in in vitro immunoassays, such as ELISA, western blot or immunohistochemical assay of disease tissue or cells. Such antibodies are also useful in isolating and purifying LCP proteins, including related cross-reactive proteins, by immunoprecipitation, immunoaffinity chromatography, or magnetic bead-mediated purification.

In a second series of antibody embodiments, the invention provides antibodies, both polyclonal and monoclonal, and fragments and derivative thereof, that bind specifically to polypeptides comprising an amino acid sequence as provided in SEQ ID NO: 1116—a 20 amino acid region of LCP2 centered about the splice junction of exons 1 and 3 of LCP1—and binding of which can be competitively inhibited by a polypeptide the sequence of which is given in SEQ ID NO: 1116 and cannot be competitively inhibited by a polypeptide having the amino acid sequence of SEQ ID NO: 3 (the full length LCP1 protein).

Such antibodies can be used to discriminate LCP2 from the LCP1 isoform and are useful in in vitro immunoassays, such as ELISA, western blot or immunohistochemical assay of disease tissue or cells. Such antibodies are also useful in isolating and purifying LCP2 proteins, including related cross-reactive proteins, by immunoprecipitation, immunoaffinity chromatography, or magnetic bead-mediated purification.

In another series of antibody embodiments, the invention provides antibodies, both polyclonal and monoclonal, and fragments and derivatives thereof, the specific binding of which can be competitively inhibited by the isolated proteins and polypeptides of the present invention.

In other embodiments, the invention further provides the above-described antibodies detectably labeled, and in yet other embodiments, provides the above-described antibodies attached to a substrate.

Pharmaceutical Compositions

LCP is important for neurological and developmental disorders, as well as diseases involving cell-cell adhesion process; defects in LCP expression, activity, distribution, localization, and/or solubility are a cause of human disease, which disease can manifest as a disorder of adrenal, adult liver, bone marrow, brain, fetal liver, heart, kidney, lung, placenta, skeletal muscle, colon or prostate function.

Accordingly, pharmaceutical compositions comprising nucleic acids, proteins, and antibodies of the present invention, as well as mimetics, agonists, antagonists, or inhibitors of LCP activity, can be administered as therapeutics for treatment of LCP defects.

Thus, in another aspect, the invention provides pharmaceutical compositions comprising the nucleic acids, nucleic acid fragments, proteins, protein fusions, protein fragments, antibodies, antibody derivatives, antibody fragments, mimetics, agonists, antagonists, and inhibitors of the present invention.

Such a composition typically contains from about 0.1 to 90% by weight of a therapeutic agent of the invention formulated in and/or with a pharmaceutically acceptable carrier or excipient.

Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X), the disclosures of which are incorporated herein by reference in their entireties, and thus need not be described in detail herein.

Briefly, however, formulation of the pharmaceutical compositions of the present invention will depend upon the route chosen for administration. The pharmaceutical compositions utilized in this invention can be administered by various routes including both enteral and parenteral routes, including oral, intravenous, intramuscular, subcutaneous, inhalation, topical, sublingual, rectal, intra-arterial, intramedullary, intrathecal, intraventricular, transmucosal, transdermal, intranasal, intraperitoneal, intrapulmonary, and intrauterine.

Oral dosage forms can be formulated as tablets, pills, dragees, capsules, liquids, gels, syrups, slurries, suspensions, and the like, for ingestion by the patient.

Solid formulations of the compositions for oral administration can contain suitable carriers or excipients, such as carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; and other agents such as acacia and alginic acid.

Agents that facilitate disintegration and/or solubilization can be added, such as the cross-linked polyvinyl pyrrolidone, agar, alginic acid, or a salt thereof, such as sodium alginate, microcrystalline cellulose, corn starch, sodium starch glycolate, and alginic acid.

Tablet binders that can be used include acacia, methylcellulose, sodium carboxymethylcellulose, polyvinylpyrrolidone (Povidone™), hydroxypropyl methylcellulose, sucrose, starch and ethylcellulose.

Lubricants that can be used include magnesium stearates, stearic acid, silicone fluid, talc, waxes, oils, and colloidal silica.

Fillers, agents that facilitate disintegration and/or solubilization, tablet binders and lubricants, including the aforementioned, can be used singly or in combination.

Solid oral dosage forms need not be uniform throughout.

For example, dragee cores can be used in conjunction with suitable coatings, such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures.

Oral dosage forms of the present invention include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Additionally, dyestuffs or pigments can be added to the tablets or dragee coatings for product identification or to characterize the quantity of active compound, i.e., dosage.

Liquid formulations of the pharmaceutical compositions for oral (enteral) administration are prepared in water or other aqueous vehicles and can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents.

The pharmaceutical compositions of the present invention can also be formulated for parenteral administration.

For intravenous injection, water soluble versions of the compounds of the present invention are formulated in, or if provided as a lyophilate, mixed with, a physiologically acceptable fluid vehicle, such as 5% dextrose (“D5”), physiologically buffered saline, 0.9% saline, Hanks' solution, or Ringer's solution.

Intramuscular preparations, e.g. a sterile formulation of a suitable soluble salt form of the compounds of the present invention, can be dissolved and administered in a pharmaceutical excipient such as Water-for-Injection, 0.9% saline, or 5% glucose solution. Alternatively, a suitable insoluble form of the compound can be prepared and administered as a suspension in an aqueous base or a pharmaceutically acceptable oil base, such as an ester of a long chain fatty acid (e.g., ethyl oleate), fatty oils such as sesame oil, triglycerides, or liposomes.

Parenteral formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like).

Aqueous injection suspensions can also contain substances that increase the viscosity of the suspension, such as sodium carboxymethyl cellulose, sorbitol, or dextran. Non-lipid polycationic amino polymers can also be used for delivery. Optionally, the suspension can also contain suitable stabilizers or agents that increase the solubility of the compounds to allow for the preparation of highly concentrated solutions.

Pharmaceutical compositions of the present invention can also be formulated to permit injectable, long-term, deposition.

The pharmaceutical compositions of the present invention can be administered topically.

A topical semi-solid ointment formulation typically contains a concentration of the active ingredient from about 1 to 20%, e.g., 5 to 10%, in a carrier such as a pharmaceutical cream base. Various formulations for topical use include drops, tinctures, lotions, creams, solutions, and ointments containing the active ingredient and various supports and vehicles. In other transdermal formulations, typically in patch-delivered formulations, the pharmaceutically active compound is formulated with one or more skin penetrants, such as 2-N-methyl-pyrrolidone (NMP) or Azone.

Inhalation formulations can also readily be formulated. For inhalation, various powder and liquid formulations can be prepared.

The pharmaceutically active compound in the pharmaceutical compositions of the present inention can be provided as the salt of a variety of acids, including but not limited to hydrochloric, sulfuric, acetic, lactic, tartaric, malic, and succinic acid. Salts tend to be more soluble in aqueous or other protonic solvents than are the corresponding free base forms.

After pharmaceutical compositions have been prepared, they are packaged in an appropriate container and labeled for treatment of an indicated condition.

The active compound will be present in an amount effective to achieve the intended purpose. The determination of an effective dose is well within the capability of those skilled in the art.

A “therapeutically effective dose” refers to that amount of active ingredient—for example LCP protein, fusion protein, or fragments thereof, antibodies specific for LCP, agonists, antagonists or inhibitors of LCP—which ameliorates the signs or symptoms of the disease or prevents progression thereof; as would be understood in the medical arts, cure, although desired, is not required.

The therapeutically effective dose of the pharmaceutical agents of the present invention can be estimated initially by in vitro tests, such as cell culture assays, followed by assay in model animals, usually mice, rats, rabbits, dogs, or pigs. The animal model can also be used to determine an initial useful concentration range and route of administration.

For example, the ED50 (the dose therapeutically effective in 50% of the population) and LD50 (the dose lethal to 50% of the population) can be determined in one or more cell culture of animal model systems. The dose ratio of toxic to therapeutic effects is the therapeutic index, which can be expressed as LD50/ED50. Pharmaceutical compositions that exhibit large therapeutic indices are particularly useful.

The data obtained from cell culture assays and animal studies is used in formulating an initial dosage range for human use, and preferably provides a range of circulating concentrations that includes the ED50 with little or no toxicity. After administration, or between successive administrations, the circulating concentration of active agent varies within this range depending upon pharmacokinetic factors well known in the art, such as the dosage form employed, sensitivity of the patient, and the route of administration.

The exact dosage will be determined by the practitioner, in light of factors specific to the subject requiring treatment. Factors that can be taken into account by the practitioner include the severity of the disease state, general health of the subject, age, weight, gender of the subject, diet, time and frequency of administration, drug combination(s), reaction sensitivities, and tolerance/response to therapy. Long-acting pharmaceutical compositions can be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Where the therapeutic agent is a protein or antibody of the present invention, the therapeutic protein or antibody agent typically is administered at a daily dosage of 0.01 mg to 30 mg/kg of body weight of the patient (e.g., 1 mg/kg to 5 mg/kg). The pharmaceutical formulation can be administered in multiple doses per day, if desired, to achieve the total desired daily dose.

Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Conventional methods, known to those of ordinary skill in the art of medicine, can be used to administer the pharmaceutical formulation(s) of the present invention to the patient. The pharmaceutical compositions of the present invention can be administered alone, or in combination with other therapeutic agents or interventions.

Therapeutic Methods

The present invention further provides methods of treating subjects having defects in LCP—e.g., in expression, activity, distribution, localization, and/or solubility of LCP—which can manifest as a disorder of adrenal, adult liver, bone marrow, brain, fetal liver, heart, kidney, lung, placenta, skeletal muscle, colon or prostate function. As used herein, “treating” includes all medically-acceptable types of therapeutic intervention, including palliation and prophylaxis (prevention) of disease.

In one embodiment of the therapeutic methods of the present invention, a therapeutically effective amount of a pharmaceutical composition comprising LCP protein, fusion, fragment or derivative thereof is administered to a subject with a clinically-significant LCP defect.

Protein compositions are administered, for example, to complement a deficiency in native LCP. In other embodiments, protein compositions are administered as a vaccine to elicit a humoral and/or cellular immune response to LCP. The immune response can be used to modulate activity of LCP or, depending on the immunogen, to immunize against aberrant or aberrantly expressed forms, such as mutant or inappropriately expressed isoforms. In yet other embodiments, protein fusions having a toxic moiety are administered to ablate cells that aberrantly accumulate LCP.

In another embodiment of the therapeutic methods of the present invention, a therapeutically effective amount of a pharmaceutical composition comprising nucleic acid of the present invention is administered. The nucleic acid can be delivered in a vector that drives expression of LCP protein, fusion, or fragment thereof, or without such vector.

Nucleic acid compositions that can drive expression of LCP are administered, for example, to complement a deficiency in native LCP, or as DNA vaccines. Expression vectors derived from virus, replication deficient retroviruses, adenovirus, adeno-associated (AAV) virus, herpes virus, or vaccinia virus can be used—see, e.g., Cid-Arregui (ed.), Viral Vectors: Basic Science and Gene Therapy, Eaton Publishing Co., 2000 (ISBN: 188129935X)—as can plasmids.

Antisense nucleic acid compositions, or vectors that drive expression of LCP antisense nucleic acids, are administered to downregulate transcription and/or translation of LCP in circumstances in which excessive production, or production of aberrant protein, is the pathophysiologic basis of disease.

Antisense compositions useful in therapy can have sequence that is complementary to coding or to noncoding regions of the LCP gene. For example, oligonucleotides derived from the transcription initiation site, e.g., between positions −10 and +10 from the start site, are particularly useful.

Catalytic antisense compositions, such as ribozymes, that are capable of sequence-specific hybridization to LCP transcripts, are also useful in therapy. See, e.g., Phylactou, Adv. Drug Deliv. Rev. 44(2-3):97-108 (2000); Phylactou et al., Hum. Mol. Genet. 7(10):1649-53 (1998); Rossi, Ciba Found. Symp. 209:195-204 (1997); and Sigurdsson et al., Trends Biotechnol. 13(8):286-9 (1995), the disclosures of which are incorporated herein by reference in their entireties.

Other nucleic acids useful in the therapeutic methods of the present invention are those that are capable of triplex helix formation in or near the LCP genomic locus. Such triplexing oligonucleotides are able to inhibit transcription, Intody et al., Nucleic Acids Res. 28(21):4283-90 (2000); McGuffie et al., Cancer Res. 60(14):3790-9 (2000), the disclosures of which are incorporated herein by reference, and pharmaceutical compositions comprising such triplex forming oligos (TFOS) are administered in circumstances in which excessive production, or production of aberrant protein, is a pathophysiologic basis of disease.

In another embodiment of the therapeutic methods of the present invention, a therapeutically effective amount of a pharmaceutical composition comprising an antibody (including fragment or derivative thereof) of the present invention is administered. As is well known, antibody compositions are administered, for example, to antagonize activity of LCP, or to target therapeutic agents to sites of LCP presence and/or accumulation.

In another embodiment of the therapeutic methods of the present invention, a pharmaceutical composition comprising a non-antibody antagonist of LCP is administered. Antagonists of LCP can be produced using methods generally known in the art. In particular, purified LCP can be used to screen libraries of pharmaceutical agents, often combinatorial libraries of small molecules, to identify those that specifically bind and antagonize at least one activity of LCP.

In other embodiments a pharmaceutical composition comprising an agonist of LCP is administered. Agonists can be identified using methods analogous to those used to identify antagonists.

In still other therapeutic methods of the present invention, pharmaceutical compositions comprising host cells that express LCP, fusions, or fragments thereof can be administered. In such cases, the cells are typically autologous, so as to circumvent xenogeneic or allotypic rejection, and are administered to complement defects in LCP production or activity.

In other embodiments, pharmaceutical compositions comprising the LCP proteins, nucleic acids, antibodies, antagonists, and agonists of the present invention can be administered in combination with other appropriate therapeutic agents. Selection of the appropriate agents for use in combination therapy can be made by one of ordinary skill in the art according to conventional pharmaceutical principles. The combination of therapeutic agents or approaches can act additively or synergistically to effect the treatment or prevention of the various disorders described above, providing greater therapeutic efficacy and/or permitting use of the pharmaceutical compositions of the present invention using lower dosages, reducing the potential for adverse side effects.

Transgenic Animals and Cells

In another aspect, the invention provides transgenic cells and non-human organisms comprising LCP isoform nucleic acids, and transgenic cells and non-human organisms with targeted disruption of the endogenous orthologue of the human LCP gene.

The cells can be embryonic stem cells or somatic cells. The transgenic non-human organisms can be chimeric, nonchimeric heterozygotes, and nonchimeric homozygotes.

Diagnostic Methods

The nucleic acids of the present invention can be used as nucleic acid probes to assess the levels of LCP mRNA in adrenal, adult liver, bone marrow, brain, fetal liver, heart, kidney, lung, placenta, skeletal muscle, colon and prostate, and antibodies of the present invention can be used to assess the expression levels of LCP proteins in adrenal, adult liver, bone marrow, brain, fetal liver, heart, kidney, lung, placenta, skeletal muscle, colon and prostate to diagnose neurological and developmental disorders, as well as diseases involving cell-cell adhesion process.

The following examples are offered for purpose of illustration, not limitation.

### EXAMPLE 1

### Identification and Characterization of cDNAs Encoding LCP Proteins

Predicating our gene discovery efforts on use of genome-derived single exon probes and hybridization to genome-derived single exon microarrays—an approach that we have previously demonstrated will readily identify novel genes that have proven refractory to mRNA-based identification efforts—we identified an exon in raw human genomic sequence that is particularly expressed in human adrenal, adult liver, bone marrow, brain, fetal liver, heart, kidney, lung, placenta and prostate, as well as a cell line, hela.

Briefly, bioinformatic algorithms were applied to human genomic sequence data to identify putative exons. Each of the predicted exons was amplified from genomic DNA, typically centering the putative coding sequence within a larger amplicon that included flanking noncoding sequence. These genome-derived single exon probes were arrayed on a support and expression of the bioinformatically predicted exons assessed through a series of simultaneous two-color hybridizations to the genome-derived single exon microarrays.

The approach and procedures are further described in detail in Penn et al., “Mining the Human Genome using Microarrays of Open Reading Frames,” Nature Genetics 26:315-318 (2000); commonly owned and copending U.S. patent application Ser. No. 09/864,761, filed May 23, 2001, Ser. No. 09/774,203, filed Jan. 29, 2001, and Ser. No. 09/632,366, filed Aug. 3, 2000, the disclosures of which are incorporated herein by reference in their entireties.

Using a graphical display particularly designed to facilitate computerized query of the resulting exon-specific expression data, as further described in commonly owned and copending U.S. patent application Ser. No. 09/864,761, filed May 23, 2001, Ser. No. 09/774,203, filed Jan. 29, 2001 and Ser. No. 09/632,366, filed Aug. 3, 2000, the disclosures of which are incorporated herein by reference in their entireties, two exons were identified that are expressed in all the human tissues tested; subsequent analysis revealed that the two exons belong to the same gene.

Table 1 summarizes the microarray expression data obtained using genome-derived single exon probes corresponding to exons two and sixteen of LCP1. Each probe was completely sequenced on both strands prior to its use on a genome-derived single exon microarray; sequencing confirmed the exact chemical structure of each probe. An added benefit of sequencing is that it placed us in possession of a set of single base-incremented fragments of the sequenced nucleic acid, starting from the sequencing primer's 3′ OH. (Since the single exon probes were first obtained by PCR amplification from genomic DNA, we were of course additionally in possession of an even larger set of single base incremented fragments of each of the single exon probes, each fragment corresponding to an extension product from one of the two amplification primers.).

Signals and expression ratios are normalized values measured and calculated as further described in commonly owned and copending U.S. patent application Ser. No. 9/864,761, filed May 23, 2001, Ser. No. 09/774,203, filed Jan. 29, 2001, Ser. No. 09/632,366, filed Aug. 3, 2000, and U.S. provisional patent application No. 60/207,456, filed May 26, 2000, the disclosures of which are incorporated herein by reference in their entireties.

TABLE 1
Expression Analysis
Genome-Derived Single Exon Microarray

| | Amplicon 24980 (exon 2 | | Amplicon 24976 (exon 16 | |
|---|---|---|---|---|
| | of LCP1) | | of LCP1) | |
| | Signal | Expression ratio | Signal | Expression ratio |
| ADRENAL | 1.23 | 1.31 | | |
| ADULT | 1.16 | −1.07 | | |
| LIVER | | | | |
| BONE | 0.93 | −1.34 | 0.46 | −1.72 |
| MARROW | | | | |
| BRAIN | 0.96 | −1.17 | 0.47 | −1.03 |
| FETAL LIVER | 1.05 | −1.09 | 0.60 | −1.24 |
| HEART | 0.99 | 1.04 | | |
| HELA | 1.43 | 1.07 | | |
| KIDNEY | 1.18 | 1.06 | 0.53 | −1.08 |
| LUNG | 1.13 | −1.11 | | |
| PLACENTA | 1.06 | −1.02 | 0.61 | 1.53 |
| PROSTATE | 1.05 | −1.01 | | |

As shown in Table 1, significant expression of exons two and sixteen of LCP1 were seen only in adrenal, adult liver, bone marrow, brain, fetal liver, heart, kidney, lung, placenta and prostate, as well as a cell line hela. Adrenal, adult liver, bone marrow, brain, fetal liver, heart, kidney, lung, placenta and prostate as well as a cell line hela-specific expression was further confirmed by RT-PCR analysis (see below).

Marathon-Ready™ liver cDNA (Clontech Laboratories, Palo Alto, Calif.) was used as a substrate for standard RACE (rapid amplification of cDNA ends) to obtain the full length cDNA clones. Oligonucleotides OL683(5′-ATGGTCCTCACTACTCTCATTCTCATATTAGTGTG-3′; SEQ ID NO: 1117) and OL684(5′-TCAAAGGATTTCTTTAAAAACATCACATTCCCC-3′; SEQ ID NO: 1118) were used to PCR out a 0.7 kb fragment of the open reading frame (ORF) using manufacture's protocols (Clontech). Oligonucleotides OL714(5′-CTGTACTAGGCCCTGAGAGTGGAACCCTTACATCC-3′; SEQ ID NO: 1119) and OL687(5′-GGGGTTTCCTCATGGTCCACTGCTTTTGCAG-3′; SEQ ID NO: 1120) were used to PCR out another 1.6 kb fragment of the ORF. Based on the sequences of these two fragments, a series of RACE reaction were performed to find out the 5′ and 3′ end of the ORF. Finally, OL759(5′-CCACCATGCCTCTGTTCCTCCTGCTCTTACTTGTCCTGC-3′; SEQ ID NO: 1121) and OL760(5′-TCAAAGGATTTCTTTAAAAACATCACATTCCCCATC-3′; SEQ ID NO: 1122) were used to PCR out the full length ORF. The RACE products were sequenced using MegaBACE™ truments (Amersham Biosciences, Sunnyvale, Calif.).

To subclone LCP into cloning vector, the RACE product generated with oligonucleotides OL759 and OL760 was ligated and T/A cloned into pGem-Teasy vector (Promega Corp.). Individual clones were picked and inserts sequenced using MegaBACE™ instruments. Two splice variants were identified following the sequence analysis of this gene. One of the cDNA clones spans 2.3 kilobases, the other one is ˜200 bp shorter. Both appear to contain long open reading frames. For reasons described below, we termed the cDNAs LCP1 and LCP2. Cloning and sequencing provided us with the exact chemical structure of the cDNA, which is shown in FIGS. 3 and 4 and further presented in the SEQUENCE LISTING as SEQ ID NO: 1 and 1113, and placed us in actual physical possession of the entire set of single-base incremented fragments of the sequenced clone, starting at the 5′ and 3′ termini.

As shown in FIG. 3, the LCP1 cDNA spans 2280 nucleotides and contains an open reading frame from nucleotide 76 through and including nt 2265 (inclusive of termination codon), predicting a protein of 729 amino acids with a (posttranslationally unmodified) molecular weight of 80.3 kD. The clone appears full length, with the reading frame opening starting with a methionine and terminating with a stop codon.

As shown in FIG. 4, the LCP2 cDNA contains an open reading frame spanning 1962 nucleotides and encodes a protein of 653 amino acids. LCP2 has a predicted molecular weight, prior to any post-translational modification, of 80.3 kD.

BLAST query of genomic sequence identified two BACS, spanning over 70 kb, that constitute the minimum set of clones encompassing the cDNA sequence. Based upon the known origin of the BACs (GenBank accession numbers AC091213.8 and AC016932.27), the LCP gene can be mapped to human chromosome 3q12.1.

Comparison of the LCP1 cDNA and genomic sequences identified 16 exons. Exon organization is listed in Table 2.

TABLE 2
LCP1 Exon Structure

| Exon no. | cDNA range | genomic range | BAC accession |
|---|---|---|---|
| 1 | 1-142 | 95410-95269 | AC091213.8 |
| 2 | 143-370 | 134826-135053 | |
| 3 | 371-508 | 9275-9138 | |
| 4 | 509-560 | 10658-10709 | AC016962.27 |
| 5 | 561-633 | 13291-13363 | |
| 6 | 634-767 | 13671-13804 | |
| 7 | 768-808 | 15742-15782 | |
| 8 | 809-1024 | 16613-16828 | |
| 9 | 1025-1149 | 18137-18261 | |
| 10 | 1150-1300 | 23549-23699 | |
| 11 | 1301-1387 | 24477-24563 | |
| 12 | 1388-1513 | 24712-24837 | |
| 13 | 1514-1607 | 27857-27950 | |
| 14 | 1608-1657 | 34382-34431 | |
| 15 | 1658-1795 | 35315-35452 | |
| 16 | 1796-2280 | 36190-36674 | |

FIG. 2 schematizes the exon organization of the LCP clones.

At the top is shown the two bacterial artificial chromosomes (BACs), with GenBank accession numbers, that span the LCP locus. The genome-derived single-exon probes first used to demonstrate expression from this locus are shown below the BACs and labeled “500”.

As shown in FIG. 2, LCP1 encodes a protein of 729 amino acids and is comprised of exons 1-16. LCP1 has a predicted molecular weight, prior to any post-translational modification of 80.3 kD. LCP2 encodes a protein of 653 amino acids and is lacking exon 2 of LCP1. LCP2 has a predicted molecular weight, prior to any post-translational modification, of 80.3 kD.

As further discussed in the examples herein, expression of LCP was assessed using hybridization to genome-derived single exon microarrays and RT-PCR. Microarray analysis of the exons two and sixteen showed expression in adrenal, adult liver, bone marrow, brain, fetal liver, heart, kidney, lung, placenta and prostate, as well as a cell line, hela. RT-PCR confirmed microarray data, and further provided expression data for skeletal muscle and colon.

The sequence of the LCP cDNAs was used as a BLAST query into the GenBank nr and dbEst databases. The nr database includes all non-redundant GenBank coding sequence translations, sequences derived from the 3-dimensional structures in the Brookhaven Protein Data Bank (PDB), sequences from SwissProt, sequences from the protein information resource (PIR), and sequences from protein research foundation (PRF). The dbEst (database of expressed sequence tags) includes ESTs, short, single pass read cDNA (mRNA) sequences, and cDNA sequences from differential display experiments and RACE experiments.

BLAST search identified multiple human and mouse ESTs, four from cow, two ESTs from pig (AW315748.1 and AW437031.1), two from chicken (AJ396784.1 and AL586285.1) and two from rat (BE098908.1 and BF543094.1) as having sequence closely related to LCP.

Globally, the N-terminal half of the human LCP1 protein resembles Neurophilin-1 precursor (NRP1), with 27% amino acid identity and 41% amino acid similarity over 443 amino acids.

Motif searches using Pfam (http://pfam.wustl.edu), SMART (http://smart.embl-heidelberg.de), and PROSITE pattern and profile databases (http://www.expasy.ch/prosite), identified several known domains.

FIG. 1 shows the domain structure of LCP1 and alignment of the identified domains with that of other proteins.

The newly isolated membrane protein LCP contains three distinct protein domains, including a CUB, an LCCL and a DSD/FA58C domain, respectively. The following four paragraphs describe the protein structure of LCP using LCP1 as an example. However, such description is also true for LCP2 except that, in comparison to LCP1, the LCP2 protein product lacks amino acid sequence 23-98 of LCP1, and therefore has a partial CUB domain the N-terminal of which is truncated. The structural features of LCP1 are schematized in FIG. 1.

LCP1 contains a CUB domain at residues 26-138 (http://smart.embl-heidelberg.de/) or alternatively at residues 26-141 (http://pfam.wustl.edu/). CUB is a protein domain with a predicted beta-barrel structure similar to that of immunoglobulins. It is an extracellular domain found in functionally diverse, mostly developmentally regulated proteins.

LCP1 has an LCCL domain at residues 147-230 (http://smart.embl-heidelberg.de/). First identified in Limulus factor C, Coch-5b2 and Lg1, the LCCL domain is hypothesized to have an antimicrobial function. Mutations in the LCCL domain have been shown to cause the deafness disorder DFNA9 in humans.

LCP1 also has a discoidin domain, also known as a F5/8 type C domain or an FA58C domain. In LCP1, the discoidin/FA58C domain occurs at residues 250-394 (discoidin domain, http://smart.embl-heidelberg.de/) or alternatively at residues 250-400 (F5/8 type C domain, http://pfam.wustl.edu/) or at residues 248-403 (FA58C, http://smart.embl-heidelberg.de/). The discoidin domain is a protein domain with a predicted amphipathic, membrane binding alpha helical structure at the C-terminal. This domain is found in a number of coagulation factors and has been shown to be responsible for phosphatidylserine-binding and essential for phosphatidylserine activity. The discoidin domain is also present in a subset of the tyrosine kinase receptor family known as discoidin domain receptors that are putatively involved in tumor progression.

The LCP1 protein contains a signal peptide consisting of the first 20 amino acid sequence of the protein. It also contains a transmembrane domain between amino acids 487 and 506 (http://www.ch.embnet.org/software/TMPRED_form.html). Other signatures of the newly isolated LCP1 proteins were identified by searching the PROSITE database (http://www.expasy.ch/tools/scnpsitl.html). These include six N-glycosylation sites (49-52, 109-112, 226-229, 428-431, 470-473, and 476-479), two cAMP- and cGMP-dependent protein kinase phosphorylation sites (313-316 and 512-515), six protein kinase C phosphorylation sites (130-132, 240-242, 279-281, 560-562, 592-594, and 654-656), thirteen casein kinase II phosphorylation sites, a single tyrosine kinase phosphorylation site (512-519), and twenty N-myristoylation sites.

Possession of the genomic sequence permitted search for promoter and other control sequences for the LCP gene. A putative transcriptional control region, inclusive of promoter and downstream elements, was defined as 1 kb around the transcription start site, itself defined as the first nucleotide of the LCP1 cDNA clone. The region, drawn from sequence of BAC AC091213.8, has the sequence given in SEQ ID NO: 42, which lists 1000 nucleotides before the transcription start site.

Transcription factor binding sites were identified using a web based program (http://motif.genome.ad.jp/), including binding sites for MZF1 (739-746 and 713-706 bp) and for GATA-1 (934-943 bp, with numbering according to SEQ ID NO: 29), amongst others.

We have thus identified a human transmembrane protein, LCP, which contains a CUB domain, a LCCL domain, and a FA58C/DS domain. The structural features strongly imply that the LCP protein plays potential therapeutic as well as diagnostic roles for neurological and developmental disorders, as well as diseases involving the cell-cell adhesion process.

### EXAMPLE 2

### Preparation and Labeling of Useful Fragments of LCP

Useful fragments of LCP are produced by PCR, using standard techniques, or solid phase chemical synthesis using an automated nucleic acid synthesizer. Each fragment is sequenced, confirming the exact chemical structure thereof.

The exact chemical structure of preferred fragments is provided in the attached SEQUENCE LISTING, the disclosure of which is incorporated herein by reference in its entirety. The following summary identifies the fragments whose structures are more fully described in the SEQUENCE LISTING:

SEQ ID NO: 1 nt, full length LCP1 cDNA

SEQ ID NO: 2 nt, cDNA ORF of LCP1

SEQ ID NO: 3 aa, full length LCP1 protein

SEQ ID NO: 4 nt, (nt 1602-1901) portion of LCP1

SEQ ID NO: 5 aa, (aa 510-608) CDS entirely within SEQ ID NO: 4

SEQ ID NO: 6 nt, (nt 2006-2280) portion of LCP1

SEQ ID NO: 7 nt, coding portion of SEQ ID NO: 6

SEQ ID NO: 8 nt, 3′ UTR portion of SEQ ID NO: 6

SEQ ID NO: 9 aa, (aa 645-729) CDS entirely within SEQ ID NO: 7

SEQ ID NO: 10-25 nt, exons 1-16 of LCP1 (from genomic sequence)

SEQ ID NO: 26-41 nt, 500 bp genomic amplicons centered about exons 1-16 of LCP1

SEQ ID NO: 42 nt, 1000 bp putative promoter of LCP

SEQ ID NOs: 43-326 nt, 17-mers scanning (nt 1602-1901) portion of LCP1

SEQ ID NOs: 327-602 nt, 25-mers scanning (nt 1602-1901) portion of LCP1

SEQ ID NOs: 603-861 nt, 17-mers scanning (nt 2006-2280) portion of LCP1

SEQ ID NOs: 862-1112 nt, 25-mers scanning (nt 2006-2280) portion of LCP1

SEQ ID NO: 1113 nt, cDNA ORF of LCP2

SEQ ID NO: 1114 aa, full length LCP2 protein

SEQ ID NO: 1115 nt, splice junction of exons 1 and 3 of LCP1 (novel junction of LCP2)

SEQ ID NO: 1116 aa, CDS within SEQ ID NO: 1115

SEQ ID NO: 1117 nt, RACE primer OL683

SEQ ID NO: 1118 nt, RACE primer OL684

SEQ ID NO: 1119 nt, RACE primer OL714

SEQ ID NO: 1120 nt, RACE primer OL687

SEQ ID NO: 1121 nt, RACE and RT-PCR primer OL759

SEQ ID NO: 1122 nt, RACE primer OL760

SEQ ID NO: 1123 nt, RT-PCR primer OL688

Upon confirmation of the exact structure, each of the above-described nucleic acids of confirmed structure is recognized to be immediately useful as an LCP-specific probe.

For use as labeled nucleic acid probes, the above-described LCP nucleic acids are separately labeled by random priming. As is well known in the art of molecular biology, random priming places the investigator in possession of a near-complete set of labeled fragments of the template of varying length and varying starting nucleotide.

The labeled probes are used to identify the LCP gene on a Southern blot, and are used to measure expression of LCP mRNA on a northern blot and by RT-PCR, using standard techniques.

### EXAMPLE 3

### LCP expression analysis by RT-PCR

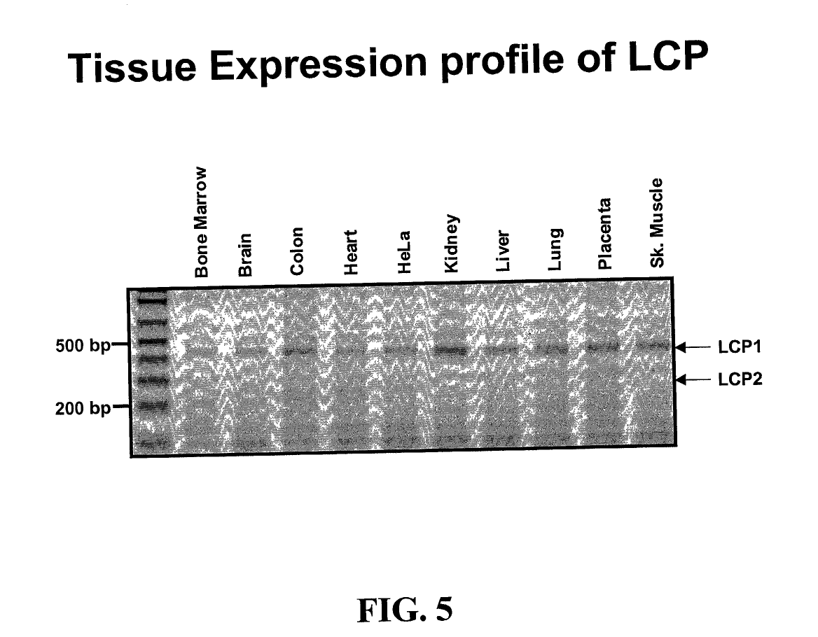

To explore the potential function of LCP, the expression of LCP in human tissues was examined by PCR using marathon-ready cDNAs. Oligonucleotides OL759 (SEQ ID NO: 1121) and OL688(5′-CTGCCCGGTCCCAGTAAGGTAAGTCATAGGTGC-3′; SEQ ID NO: 1123) were used to amplify a 5′ fragment from LCP from human cDNAs of bone marrow, brain, colon, heart, kidney, liver, lung, placenta, skeletal muscle and Hela cells. The PCR conditions were according to a touchdown PCR procedure. The tubes containing the oligonucleotides, cDNA and Taq polymerase were first incubated at 94° C. for 15 seconds followed by 70° C. for 2 minutes, cycle 5 times. The tubes were then incubated at 94° C. for 15 seconds followed by 68° C. for 2 minutes, cycle 5 times. Finally the tubes were incubated at 94° C. for 15 seconds followed by 66° C. for 2 minutes, cycle 25 times. To distinguish the two splice variants, the PCR fragments were cut by restriction enzyme BglII and the rsulting mixture was run on an agarose gel. The sizes of the fragment expected from LCP1 is 435 bp and from LCP2 is 207 bp. The result of the expression profile is shown in FIG. 5. The abundance of PCR product indicates that LCP1 is expressed in all tissues examined with the highest expression in kidney. LCP2 is not detectable in all tissues except a very slight expression in kidney. Therefore, LCP1 is the dominant form of LCP expression and may play the major role for LCP function.

### EXAMPLE 4

### Production of LCP Protein

The full length LCP1 or LCP2 cDNA clone is cloned into the mammalian expression vector pcDNA3.1/HISA (Invitrogen, Carlsbad, Calif., USA), transfected into COS7 cells, transfectants selected with G418, and protein expression in transfectants confirmed by detection of the anti-Xpress™ epitope according to manufacturer's instructions. Protein is purified using immobilized metal affinity chromatography and vector-encoded protein sequence is then removed with enterokinase, per manufacturer's instructions, followed by gel filtration and/or HPLC.

Following epitope tag removal, LCP protein is present at a concentration of at least 70%, measured on a weight basis with respect to total protein (i.e., w/w), and is free of acrylamide monomers, bis acrylamide monomers, polyacrylamide and ampholytes. Further HPLC purification provides LCP protein at a concentration of at least 95%, measured on a weight basis with respect to total protein (i.e., w/w).

### EXAMPLE 5

### Production of Anti-LCP Antibody

Purified proteins prepared as in Example 4 are conjugated to carrier proteins and used to prepare murine monoclonal antibodies by standard techniques. Initial screening with the unconjugated purified proteins, followed by competitive inhibition screening using peptide fragments of the LCP, identifies monoclonal antibodies with specificity for LCP.

### EXAMPLE 6

### Use of LCP Probes and Antibodies for Diagnosis

After informed consent is obtained, samples are drawn from disease tissue or cells and tested for LCP mRNA levels by standard techniques and tested additionally for LCP protein levels using anti- LCP antibodies in a standard ELISA.

### EXAMPLE 7

### Use of LCP Nucleic Acids, Proteins, and Antibodies in Therapy

Once over-expression of LCP is detected in patients, LCP specific antisense RNA or LCP-specific antibody is introduced into disease cells to reduce the amount of the protein.

Once mutations of LCP have been detected in patients, normal LCP is reintroduced into the patient's disease cells by introduction of expression vectors that drive LCP expression or by introducing LCP proteins into cells. Antibodies for the mutated forms of LCP are used to block the function of the abnormal forms of the protein.

### EXAMPLE 8

### Human LCP Disease Associations

Diseases that map to the human LCP chromosomal region are shown in Table 3:

TABLE 3
Diseases mapped to human chromosome 3q12.1 (LCP region).

| | | chromosomal |
|---|---|---|
| mim_num | disease | location |
| 601869 | Deafness, autosomal | 3q |
| | recessive 15 | |
| 602668 | Mytonic dystrophy 2 | 3q |

At least one of these diseases has physiological characteristics consistent with alteration of an LCP-like gene.

For example, a number of recent studies have shown that mutations in the LCCL domain of a gene named COCH cause a autosomal recessive deafness disorder, DFNA9 (Robertson N. G. et al, Nature Genetics 20:299-302 (1998)). LCP is therefore a candidate gene for the deafness syndrome, autosomal recessive 15.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

What is claimed is:

1. An isolated nucleic acid that encodes a protein involved in neurological and developmental disorders, as well as diseases involving cell-cell adhesion process, comprising:
(a) a nucleotide sequence selected from the group consisting of:
(i) SEQ ID NO: 1;
(ii) the complement of the sequences set forth in (i);
(iii) the nucleotide sequence of SEQ ID NO: 2, 1113;
(iv) a degenerate variant of the sequences set forth in (iii); and
(v) the complement of the sequences set forth in (iii) and (iv); or
(b) a nucleotide sequence selected from the group consisting of:
(i) a nucleotide sequence that encodes a polypeptide having the sequence of SEQ ID NO: 3, 1114;
(ii) a nucleotide sequence that encodes a polypeptide having the sequence of SEQ ID NO: 3, 1114, with conservative amino acid substitutions; and
(iii) the complement of the sequences set forth in (i) and (ii),
wherein said isolated nucleic acid comprising a nucleotide sequence selected from group (b) is no more than about 100 kb in length.

2. The isolated nucleic acid of claim 1 wherein said nucleic acid, or the complement of said nucleic acid, encodes a polypeptide involved in neurological and developmental disorders, as well as diseases involving cell-cell adhesion process.

3. The isolated nucleic acid of claim 1, wherein said nucleic acid, or the complement of said nucleic acid, is expressed in adrenal, adult liver, bone marrow, brain, fetal liver, heart, kidney, lung, placenta, skeletal muscle, colon and prostate, as well as a cell line, hela.

4. A nucleic acid probe, comprising:
(a) the nucleic acid of claim 1; or
(b) at least 17 contiguous nucleotides of SEQ ID Nos: 4, 6 or 1115,
wherein said probe according to (b) is no longer than about 100 kb in length.

5. The probe of claim 4, wherein said probe is detectably labeled.

6. The probe of claim 4, attached to a substrate.

7. A microarray, wherein at least one probe of said array is a probe according to claim 4.

8. The isolated nucleic acid molecule of claim 1, wherein said nucleic acid molecule is operably linked to one or more expression control elements.

9. A replicable vector comprising a nucleic acid molecule of claim 1.

10. A replicable vector comprising an isolated nucleic acid molecule of claim 8.

11. A host cell transformed to contain the nucleic acid molecule of any one of claim 1 or 8-10, or the progeny thereof.

12. A method for producing a polypeptide, the method comprising: culturing the host cell of claim 11 under conditions in which the protein encoded by said nucleic acid molecule is expressed.

13. An isolated polypeptide produced by the method of claim 12.

14. An isolated polypeptide, comprising:
(a) an amino acid sequence selected from the group consisting of SEQ ID NO: 3 and 1114;
(b) an amino acid sequence having at least 65% amino acid sequence identity to that of (a)(i) or (a)(ii);
(c) an amino acid sequence according to (a)(i) or (a)(ii) in which at least 95% of deviations from the sequence of (a)(i) or (a)(ii) are conservative substitutions; or
(d) a fragment of at least 8 contiguous amino acids of any of (a)-(c).

15. A fusion protein, said fusion protein comprising a polypeptide of claim 14 fused to a heterologous amino acid sequence.

16. The fusion protein of claim 15, wherein said heterologous amino acid sequence is a detectable moiety.

17. The fusion protein of claim 16, wherein said detectable moiety is fluorescent.

18. The fusion protein of claim 15, wherein said heterologous amino acid sequence is an Ig Fc region.

19. An isolated antibody, or antigen-binding fragment or derivative thereof, the binding of which can be competitively inhibited by a polypeptide of claim 14.

20. A transgenic non-human animal modified to contain the nucleic acid molecule of any one of claim 1 or 8-10.

21. A transgenic non-human animal unable to express the endogenous orthologue of the nucleic acid molecule of claim 1.

22. A method of identifying agents that modulate the expression of human LCP, the method comprising:
contacting a cell or tissue sample believed to express human LCP with a chemical or biological agent, and then comparing the amount of human LCP expression in said cell or tissue sample with that of a control,
changes in the amount relative to control identifying an agent that modulates expression of human LCP.

23. A method of identifying agonists and antagonists of human LCP, the method comprising:
contacting a cell or tissue sample believed to express human LCP with a chemical or biological agent, and then comparing the activity of human LCP with that of a control,
increased activity relative to a control identifying an agonist, decreased activity relative to a control identifying an antagonist.

24. A purified agonist of the polypeptide of claim 14.

25. A purified antagonist of the polypeptide of claim 14.

26. A method of identifying a specific binding partner for a polypeptide according to claim 14, the method comprising:
contacting said polypeptide to a potential binding partner; and
determining if the potential binding partner binds to said polypeptide.

27. The method of claim 26, wherein said contacting is performed in vivo.

28. A purified binding partner of the polypeptide of claim 14.

29. A method for detecting a target nucleic acid in a sample, said target being a nucleic acid according to claim 1, the method comprising:
(a) hybridizing the sample with a probe comprising at least 17 contiguous nucleotides of a sequence complementary to said target nucleic acid in said sample under high stringency hybridization conditions, and
(b) detecting the presence or absence, and optionally the amount, of said binding.

30. A method of diagnosing a disease caused by mutation in human LCP, comprising:
detecting said mutation in a sample of nucleic acids that derives from a subject suspected to have said disease.

31. A method of diagnosing or monitoring a disease caused by altered expression of human LCP, comprising:
determining the level of expression of human LCP in a sample of nucleic acids or proteins that derives from a subject suspected to have said disease,
alterations from a normal level of expression providing diagnostic and/or monitoring information.

32. A diagnostic composition comprising the nucleic acid of claim 1, said nucleic acid being detectably labeled.

33. The diagnostic composition of claim 32, wherein said composition is further suitable for in vivo administration.

34. A diagnostic composition comprising the polypeptide of claim 14, said polypeptide being detectably labeled.

35. The diagnostic composition of claim 34, wherein said composition is further suitable for in vivo administration.

36. A diagnostic composition comprising the antibody, or antigen-binding fragment or derivative thereof, of claim 19.

37. The diagnostic composition of claim 36, wherein said antibody or antigen-binding fragment or derivative thereof is detectably labeled.

38. The diagnostic composition of claim 37, wherein said composition is further suitable for in vivo administration.

39. A pharmaceutical composition comprising the nucleic acid of claim 1 and a pharmaceutically acceptable excipient.

40. A pharmaceutical composition comprising the polypeptide of claim 14 and a pharmaceutically acceptable excipient.

41. A pharmaceutical composition comprising the antibody or antigen-binding fragment or derivative thereof of claim 19 and a pharmaceutically acceptable excipient.

42. A pharmaceutical composition comprising the agonist of claim 24 and a pharmaceutically acceptable excipient.

43. A pharmaceutical composition comprising the antagonist of claim 25 and a pharmaceutically acceptable excipient.

44. A method for treating or preventing a disorder associated with decreased expression or activity of human LCP, the method comprising administering to a subject in need of such treatment an effective amount of the pharmaceutical composition of any of claim 39, 40 or 42.

45. A method for treating or preventing a disorder associated with increased expression or activity of human LCP, the method comprising administering to a subject in need of such treatment an effective amount of the pharmaceutical composition of claim 41 or 43.

46. A method of modulating the expression of a nucleic acid according to claim 1, the method comprising:
administering an effective amount of an agent which modulates the expression of a nucleic acid according to claim 1.

47. A method of modulating at least one activity of a polypeptide according to claim 14, the method comprising:
administering an effective amount of an agent which modulates at least one activity of a polypeptide according to claim 14.
